(19) United States
(12) Patent Application Publication
(10) Pub. No.: US 2014/0348863 A1
(43) Pub. Date: Nov. 27, 2014

(54) CMV ANTIGENS AND USES THEREOF

(71) Applicants: Alessia Bianchi, Siena (IT); Luca Bruno, Siena (IT); Stefano Calo, Siena (IT); Mirko Cortese, Siena (IT); Tobias Kessler, Kundl / Tirol (AT); Marcello Merola, Siena (IT); Yasushi Uematsu, Siena (IT); NOVARTIS AG, Basel (CH)

(72) Inventors: Alessia Bianchi, Florence (IT); Luca Bruno, Siena (IT); Stefano Calo, Siena (IT); Mirko Cortese, Siena (IT); Tobias Kessler, Boms (DE); Marcello Merola, Monticiano (IT); Yasushi Uematsu, Siena (IT)

(21) Appl. No.: 14/350,988

(22) PCT Filed: Oct. 11, 2012
(86) PCT No.: PCT/IB2012/002491

§ 371 Date: Apr. 10, 2014

Related U.S. Application Data
(60) Provisional application No. 61/546,150, filed on Oct. 12, 2011.

(51) Int. Cl. C07K 14/005; C12N 7/00

(52) U.S. Cl. CPC C07K 14/005; C12N 7/00; A61K 39/245
USPC 424/186.1; 530/350; 536/23.72; 514/44 R; 435/320.1; 435/69.3; 435/375

(57) ABSTRACT

The present invention relates to immunogenic compositions comprising CMV antigens and methods for preparing compositions that contain CMV antigens. The invention also relates to methods for inducing an immune response to CMV.

### SEQUENCE LISTING

The instant application contains a Sequence Listing which has been submitted in ASCII format via EFS-Web and is hereby incorporated by reference in its entirety. Said ASCII copy, created on Sep. 28, 2012, is named PAT054443.txt and is 157,963 bytes in size.

### BACKGROUND

Human cytomegalovirus (HCMV) causes widespread, persistent human infections that vary with the age and immunocompetence of the host. It can remain latent throughout the lifetime of the host with sporadic reactivation events. The primary infection of hosts with a functional immune system is associated with mild symptoms although it may progress with fever, hepatitis, splenomegaly and a mononucleosis-like disease. In contrast, when primary infection or reactivation occurs in immunocompromised or immunodeficient hosts, they often experience life-threatening diseases, including pneumonia, hepatitis, retinitis and encephalitis (Sinclair and Sissons, J. Gen. Virol. 87:1763-1779, 2006). HCMV infection has been recognized for its association with three different populations: neonates with immature immune systems; transplant recipients with impaired immune systems due to the use of drugs and HIV-infected patients with compromised immune systems due to the decline of CD4⁺ T cells.

HCMV can be particularly devastating in neonates, causing defects in neurological development. In the industrialized countries, intrauterine viral infection is most common. Estimates suggest that between 0.6% and 0.7% (depending on the seroprevalence of the population examined) of all new neonates are infected in utero (Dollard et al., Rev. Med. Virol., 17(5):355-363, 2007). In the United States alone, this corresponds to approximately 40,000 new infections each year. Around 1.4% of intrauterine CMV infections occur from transmission by women with established infection. New maternal infection occurs in 0.7 to 4.1% of pregnancies and is transmitted to the fetus in about 32% of cases. Around 90% of infected infants are asymptomatic at birth and most will develop serious consequences of the infection over the course of several years, including mental retardation and hearing loss. Other infected children show symptomatic HCMV disease with symptoms of irreversible central nervous system involvement in the form of microencephaly, encephalitis, seizures, deafness, upper-motor neuron disorders and psychomotor retardation (Kenneson et al., Rev. Med. Virol., 17(4):253-276, 2007). In sum, approximately 8,000 children in the United States develop virus-related neurological disease each year. Congenital infection is the major driving force behind efforts to develop an HCMV vaccine.

Efforts to develop a HCMV vaccine began more than 40 years ago. Over the years a number of HCMV vaccines have been evaluated, including a whole virus vaccine, chimeric vaccines and subunit vaccines. The whole virus vaccine neither prevented infection or vial reactivation in immunized adult women, nor increased protection against diseases compared to seropositive individuals (Arvin et al., Clin. Infect. Dis. 39(2), 233-239, 2004). Each of the chimeric vaccines were well tolerated, but concerns about the potential risk of establishing a latent infection hindered the progression of those vaccines. The subunit vaccine approach, based on the assumption that immunity directed toward a limited number of dominant antigens, has showed low efficacy thus far. These results suggest that an effective vaccine may need to be directed towards multiple antigens expressed at different stages of viral replication.

Thus, a need exists for immunogenic compositions comprising one or more CMV proteins and for immunization methods that produce better immune responses.

### SUMMARY OF THE INVENTION

The invention relates to immunogenic compositions that comprise one or more human cytomegalovirus (CMV) polypeptides selected from the group consisting of RL10, RL11, RL12, RL13, UL5, UL80.5, UL116, UL119, UL122, UL132, UL133, UL138, UL139, UL148A, and fragments thereof. Optionally, the one or more human CMV polypeptides are selected from the group consisting of RL11, RL13 and UL119. The human CMV polypeptides can be RL11 and UL119. Optionally, the immunogenic compositions can further comprise an adjuvant. The adjuvant can be alum, MF59, IC31, Eisai 57, ISCOM, CpG, or pet lipid A.

The invention also relates to immunogenic compositions that comprise two or more human CMV polypeptides selected from the group consisting of RL10, RL11, RL12, RL13, UL5, UL80.5, UL116, UL119, UL122, UL132, UL133, UL138, UL139, UL148A and fragments thereof. The two or more human CMV polypeptides are selected from the group consisting of RL11, RL13, and UL119. The two CMV polypeptides can be RL11 and UL119.

The invention also relates to recombinant human CMV polypeptides and isolated nucleic acids encoding one or more human CMV polypeptides selected from the group consisting of RL10, RL11, RL12, RL13, UL5, UL80.5, UL116, UL119, UL122, UL132, UL133, UL138, UL139, UL148A and fragments thereof. The isolated nucleic acid can be self replicating RNA. Preferably the self replicating RNA is an alphavirus replicon.

The invention also relates to an alphavirus replication particle (VRP) comprising an alphavirus replicon. An immunogenic composition may comprise the VRP.

The invention also relates to a method of inducing an immune response in an individual, comprising administering to the individual an immunogenic composition, a nucleic acid, or a VRP as described herein. The immune response can comprise the production of neutralizing anti-CMV antibodies. The neutralizing antibodies can be complement-independent.

The invention further relates to a method of forming a CMV protein complex, comprising delivering nucleic acids encoding two or more CMV proteins selected from the group consisting of RL10, RL11, RL12, RL13, UL5, UL80.5, UL116, UL119, UL122, UL132, UL133, UL138, UL139, and UL148A to a cell, and maintaining the cell under conditions suitable for expression of the first CMV protein and the second CMV protein, wherein a CMV protein complex is formed. The cell can be in vivo. The cell can be an epithelial cell, an endothelial cell, or a fibroblast.

The invention also relates to a method of inhibiting CMV entry into a cell, comprising contacting the cell with an immunogenic composition or an immunogenic complex described herein.

### BRIEF DESCRIPTION OF THE DRAWINGS

FIG. 1 is a sequence alignment of RL13 from Merlin (SEQ ID NO: 87) and TB40E (SEQ ID NO: 88) strains. Conserved residues are embedded in a blue box. N-linked glycosylation are indicated by and “*”. Transmembrane and signal peptide are enclosed respectively in a yellow and a green box, while immunoglobulin superfamily domain (IgSF) is enclosed in the red box.

FIG. 2 shows Western blot analysis on protein extracts of ARPE-19 cells transfected with: 1) pcDNA3.1_RL10; 2) pcDNA3.1_RL11; 3) pcDNA3.1_RL13; 4) pcDNA3.1_UL119; 5) pcDNA3.1. Membrane was probed with non-immune hIgG (FIG. 2A) and then stripped and re-probed with anti-His antibody. The “*” indicated the bands present in both FIG. 2A and FIG. 2B.

FIG. 3 shows deglycosylase treatment of RL13. Cell lysates of ARPE-19 transiently expressing RL13 were incubated with buffer only (U), PNGaseF (F) and N-glycosylase, sialidase and O-glycosylase (O) enzymes. The untreated sample shows 3 bands of approximately 70 kDa, 98 kDa, and 140 kDa. Upon treatment with PNGaseF, the 100 kDa form migrates at 55 kDa, while the 70 kDa undergoes complete deglycosylation reaching a Mw of 37 kDa.

FIG. 4A shows RL11, RL12 and RL13 are able to bind the Fc portion of immunoglobulins while signals retrieved from RL10 and gB are comparable to the negative control. HEK 293T cells expressing myc tagged gB, RL10, RL11, RL12, RL13 and mock transfected were fixed, permeabilized and stained using both anti-myc FITC conjugated and human IgG Fc fragment (hFc) Alexa fluor 647 conjugated. FITC positive cells were compared to mock transfected cells for their ability to bind hFc. FIG. 4B shows that RL13 binds different IgG subclasses. HEK 293T cells were transiently transfected with myc tagged RL11, RL13 and empty vector. Cells were fixed, permeabilized and stained using different human immunoglobulin subclasses or Fc fragment of total IgG. While RL11 binds with equal efficiency all of the tested isotypes, RL13 exhibits signal only in the presence of IgG1 and IgG2 with higher signals for the latter.

FIG. 5 shows RL13 intracellular localization and human IgG Fc binding. ARPE19 epithelial cells were transfected with RL13-YFP fusion protein (central column). Cells were fixed, permeabilized and stained with antibodies against different intracellular compartments (second column) and with a fluorophore conjugated human IgG Fc fragment (fourth column). Cells were then observed with a confocal microscope. Confocal section of representative cells are shown: the merge panel shows a partial colocalization between RL13 and markers of golgi, trans-golgi and early endosomes (first column), while Fc signal perfectly colocalizes with RL13 (last column, merge).

FIG. 6 shows HCMV RL13 is internalized upon binding of human IgG Fc portion into mature endosomes through clathrin mediated endocytosis. ARPE-19 epithelial cells were transfected with RL13. Cells were incubated at 4° C. with a fluorophore conjugated human IgG Fc fragment and then fixed at different time points after incubation at 37° C. Images and Z-stacks were collected with a confocal microscope. Orthogonal projection of Z-stack of two different time points are shown. (A) Upon binding to the surface of transfected cells, human Fc signal is retrieved in cell membrane clusters that colocalize with RL13 signals (merge panel, indicated with arrows). (B) Thirty minutes after incubation at 37° C. the RL13-human Fc complex is internalized and accumulates (C) in vesicles for early endosomes marker (Rab5).

FIG. 7A is a flowchart of RL13 immunoprecipitation. Cells expressing RL13(+) and control cells (−) were incubated at 4° C. with a biotinylated human Fc fragment. Cells were then transferred to 37° C. and after 1 hour incubation they were harvested and lysed. Streptavidin-conjugated beads were added to the lysate to precipitate the hFc-RL13 complex. Elution and total lysate were loaded on SDS-PAGE, blotted and probed using anti-RL13 and anti-human Fc antibodies. FIG. 7B shows a Western blot on elution and total lysate fractions. Signal of the human Fc fragment is retrieved only in the RL13 transfected sample (+lane, lower panel). As expected, RL13 is present in the elution fraction (upper panel), thus confirming it binds to the Fc portion of immunoglobulin.

FIG. 8 shows acceptor photobleach FRET analysis of UL119 and RL11. Intensity images of RL11-CFP (CI and CII) and UL-119-YFP (YI and YII) are shown. CI and YI indicates the fluorescence intensity distribution before the bleaching event. UL119-YFP was subsequently photobleached in a specific segment (white box), thereby eliminating energy transfer. Then a second donor fluorescence image (CII) was taken. YII indicates the fluorescence intensity distribution of UL119-YFP after photobleaching. CII shows the fluorescence intensity distribution of RL11-CFP after photobleaching of the acceptor, and the resulting brightening of the selected area.

FIG. 9 is a graph showing quantification of FRET efficiencies. The indicated number of cells (n) were analyzed in two different experiments, and the calculated FRET efficiency is given as plot distribution. Negative control (YFP and CFP proteins alone) is also shown. Positivity threshold value of 10% is indicated by a line. As shown UL119 and RL11 pairs are high above the threshold value demonstrating their interaction to form a complex.

FIG. 10 shows only UL119 co-elutes with RL11 (right panel “Elution”, sample A), confirming the interaction between these two proteins. HEK293T cells were co-transfected with different plasmids (A=293T cotransfected w/ UL119 myc & RL11 his; B=293 t contransfected w/ RL10myc & RL11 his; C=293T contransfected w/ UL138myc & RL11his; D=293T cotransfected w/ UL80.5myc & RL11 his; E=293T cotransfected w/ UL122myc & RL11 his; F=293T cotransfected w/ YFPmyc & RL11 his). Immunoprecipitation was performed with anti-histidine tag antibodies and western blot analysis was carried out with both anti-myc antibodies (right panel), to reveal the co-immunoprecipitated interactors, and anti-his antibody (left panel) to confirm the presence of RL11.

FIG. 11 shows both UL119 and RL11 proteins are present in the envelope fraction, demonstrating they are both present on the surface of the virus. Purified HCMV virus was collected from infected cells supernatant and detergent extracted. Tegument and capsid proteins (Tc) were separated from envelope proteins (E). Fractions were analyzed through western blot using specific anti-sera for the respective proteins.

### DETAILED DESCRIPTION

As described and exemplified herein, the inventors have discovered new human cytomegalovirus (CMV) antigens. Thus, the invention provides immunogenic compositions comprising CMV proteins and fragments thereof, nucleic acids encoding CMV and fragments thereof, or viral vectors that contain CMV proteins or fragments thereof, and methods for producing an immunogenic response in individuals, comprising administering a CMV immunogenic composition to an individual in need thereof.

In a general aspect, the invention relates to immunogenic compositions for delivery of one or more CMV antigens to a subject. The immunogenic compositions may comprise a CMV polypeptide or protein, nucleic acids encoding a CMV protein (e.g., DNA, self-replicating RNA molecules, non self-replicating RNA molecules), or a viral vector encoding CMV protein. The CMV polypeptide may be a CMV polypeptide described in this application, or any one of the known CMV polypeptides, including, for example, a CMV Tier 1 polypeptide, such as gB, gH, gL; gO; gM, gN; UL128, UL130, or UL131.

In another aspect, the immunogenic compositions may comprise one or more recombinant nucleic acid molecules that contain a first sequence encoding a first CMV protein or fragment thereof, and optionally, a second sequence encoding a second CMV protein or fragment thereof. The recombinant nucleic acid molecules may encode any one of the CMV proteins described herein, or fragments thereof, or may be any one of the known CMV proteins, including, for example, a CMV Tier 1 protein such as gB, gH, gL; gO; gM, gN; UL128, UL130, or UL131. If desired, one or more additional sequences encoding additional proteins, for example, a third CMV protein or fragment thereof, a fourth CMV protein or fragment thereof, a fifth CMV protein or fragment thereof etc., can be present in the recombinant nucleic acid molecule. In some aspects, the CMV proteins form an immunogenic complex. The sequences encoding CMV proteins or fragments thereof are operably linked to one or more suitable control elements so that the CMV proteins or fragments are produced by a cell that contains the recombinant nucleic acid.

In one embodiment, an immunogenic composition of the invention comprises one or more human CMV polypeptides selected from the group consisting of RL10, RL11, RL12, RL13, UL5, UL80.5, UL116, UL122, UL132, UL133, UL138, UL139, UL148A, and fragments thereof.

In one embodiment, an immunogenic composition of the invention comprises one or more human CMV polypeptides selected from the group consisting of gB, gH, gL; gO; gM, gN; UL128, UL130, UL131 and fragments thereof and one or more human CMV polypeptides selected from the group consisting of RL10, RL11, RL12, RL13, UL5, UL80.5, UL116, UL122, UL132, UL133, UL138, UL139, UL148A and fragments thereof.

In another embodiment, an immunogenic composition of the invention comprises RL10 and one or more human CMV polypeptides selected from the group consisting of gB, gH, gL; gO; gM, gN; UL128, UL130, UL131 and fragments thereof.

In another embodiment, an immunogenic composition of the invention comprises RL11 and one or more human CMV polypeptides selected from the group consisting of gB, gH, gL; gO; gM, gN; UL128, UL130, UL131 and fragments thereof.

In another embodiment, an immunogenic composition of the invention comprises RL12 and one or more human CMV polypeptides selected from the group consisting of gB, gH, gL; gO; gM, gN; UL128, UL130, UL131 and fragments thereof.

In another embodiment, an immunogenic composition of the invention comprises RL13 and one or more human CMV polypeptides selected from the group consisting of gB, gH, gL; gO; gM, gN; UL128, UL130, UL131 and fragments thereof.

In another embodiment, an immunogenic composition of the invention comprises UL5 and one or more human CMV polypeptides selected from the group consisting of gB, gH, gL; gO; gM, gN; UL128, UL130, UL131 and fragments thereof.

In another embodiment, an immunogenic composition of the invention comprises UL80.5 and one or more human CMV polypeptides selected from the group consisting of gB, gH, gL; gO; gM, gN; UL128, UL130, UL131 and fragments thereof.

In another embodiment, an immunogenic composition of the invention comprises UL116 and one or more human CMV polypeptides selected from the group consisting of gB, gH, gL; gO; gM, gN; UL128, UL130, UL131 and fragments thereof.

In another embodiment, an immunogenic composition of the invention comprises UL119 and one or more human CMV polypeptides selected from the group consisting of gB, gH, gL; gO; gM, gN; UL128, UL130, UL131 and fragments thereof.

In another embodiment, an immunogenic composition of the invention comprises UL122 and one or more human CMV polypeptides selected from the group consisting of gB, gH, gL; gO; gM, gN; UL128, UL130, UL131 and fragments thereof.

In another embodiment, an immunogenic composition of the invention comprises UL132 and one or more human CMV polypeptides selected from the group consisting of gB, gH, gL; gO; gM, gN; UL128, UL130, UL131 and fragments thereof.

In another embodiment, an immunogenic composition of the invention comprises UL133 and one or more human CMV polypeptides selected from the group consisting of gB, gH, gL; gO; gM, gN; UL128, UL130, UL131 and fragments thereof.

In another embodiment, an immunogenic composition of the invention comprises UL138 and one or more human CMV polypeptides selected from the group consisting of gB, gH, gL; gO; gM, gN; UL128, UL130, UL131 and fragments thereof.

In another embodiment, an immunogenic composition of the invention comprises UL139 and one or more human CMV polypeptides selected from the group consisting of gB, gH, gL; gO; gM, gN; UL128, UL130, UL131 and fragments thereof.

In another embodiment, an immunogenic composition of the invention comprises UL148A and one or more human CMV polypeptides selected from the group consisting of gB, gH, gL; gO; gM, gN; UL128, UL130, UL131 and fragments thereof.

### CMV Antigens

Suitable CMV antigens include the CMV polypeptides RL10, RL11, RL12, RL13, UL5, UL80.5, UL116, UL119, UL122, UL132, UL133, UL138, UL139, UL148A, or fragments thereof, or proteins having sequence similarity to RL10, RL11, RL12, RL13, UL5, UL80.5, UL116, UL119, UL122, UL132, UL133, UL138, UL139, UL148A, or fragments thereof, and can be from any CMV strain. For example, CMV proteins can be from Merlin, AD 169, VR1814, Towne, Toledo, TR, PH, TB40/e, or Fix (alias VR1814) strains of CMV. Exemplary CMV proteins and fragments are described herein. These proteins and fragments can be encoded by any suitable nucleotide sequence, including sequences that are codon optimized or deoptimized for expression in a desired host, such as a human cell. Typically the CMV protein will have at least 75% identity, at least 80%, at least 85%, at least 90%, at least 95%, at least 97%, at least 98%, or at least 99%, identity to the amino acid sequence of RL10, RL11, RL12, RL13, UL5, UL80.5, UL116, UL119, UL122, UL132, UL133, UL138, UL139, UL148A or a fragment thereof. Amino acid sequence identity is preferably determined using a suitable sequence alignment algorithm and default parameters, such as BLASTP and BLASTX from the package BLAST version 2.2.18 provided by the NCBI, National Center for Biotechnology Information (Altschul, S. F., Gish, W., Miller, W., Myers, E. W. & Lipman, D. J. (1990) “Basic local alignment search tool.” J. Mol. Biol. 215:403-410). Typically, the CMV nucleic acids will have at least 75% identity, at least 80%, at least 85%, at least 90%, at least 95%, at least 97%, at least 98%, or at least 99%, identity to the nucleic acid sequence of RL10, RL11, RL12, RL13, UL5, UL80.5, UL116, UL119, UL122, UL132, UL133, UL138, UL139 or UL148A. BLASTN and TBLASTN programs for determining nucleotide sequence identity are available from the same package. Protein sequence alignments are available using FASTA35 and SSEARCH programs from the package fasta version 35.4.3 (Improved tools for biological sequence comparison. Pearson W R, Lipman D J. Proc Natl Acad Sci USA. 1988 April; 85(8):2444-8. PMID: 3162770). ClustalW version 2.0.10 (Multiple sequence alignment with the Clustal series of programs. (2003) Chema, Ramu, Sugawara, Hideaki, Koike, Tadashi, Lopez, Rodrigo, Gibson, Toby J, Higgins, Desmond G, Thompson, Julie D. Nucleic Acids Res 31 (13):3497-500 PMID: 12824352) is available for multiple protein sequence alignments.

RL10 Proteins

A RL10 protein (alternatively known as TRL10, gpTRL10) can be full length or can omit one or more regions of the protein. Alternatively, fragments of a RL10 protein can be used. RL10 amino acids are numbered according to the full-length RL10 amino acid sequence (CMV RL10 FL) shown in SEQ ID NO: 8, which is 170 amino acids long. Optionally, the RL10 protein can be a RL10 fragment of 10 amino acids or longer. For example, the number of amino acids in the fragment can comprise 10, 15, 20, 30, 40, 50, 60, 70, 80, 90, 100, 125, 150, or 160 amino acids. A RL10 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160 and/or terminate at residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169 or 170.

Optionally, a RL10 fragment can extend further into the N-terminus by 5, 10, 20, or 30 amino acids from the starting residue of the fragment. Optionally, a RL10 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of RL10 or fragment thereof.

RL10 is an envelope glycoprotein and is dispensable for viral replication.

RL11 Protein

A RL11 protein (alternatively known as gp34) can be full length or can omit one or more regions of the protein. Alternatively, fragments of a RL11 protein can be used. RL11 amino acids are numbered according to the full-length RL11 amino acid sequence (CMV RL11 FL) shown in SEQ ID NO: 14, which is 234 amino acids long. Optionally, the RL11 protein can be a RL11 fragment of 10 amino acids or longer. For example, the number of amino acids in the fragment can comprise 10, 15, 20, 30, 40, 50, 60, 70, 80, 90, 100, 125, 150, 175, 200, or 225 amino acids. A RL11 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, or 224 and/or terminate at residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233 or 234.

Optionally, a RL11 fragment can extend further into the N-terminus by 5, 10, 20, or 30 amino acids from the starting residue of the fragment. Optionally, a RL11 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of RL11 or fragment thereof.

RL11 is a membrane-associated glycoprotein. RL11 is a known Fc binding protein and can form complexes with UL119 (See Example 6 and 7).

RL12 Proteins

A RL12 protein can be full length or can omit one or more regions of the protein. Alternatively, fragments of a RL12 protein can be used. RL12 amino acids are numbered according to the full-length RL12 amino acid sequence (CMV RL12 FL) shown in SEQ ID NO: 18, which is 410 amino acids long. Optionally, the RL12 protein can be a RL12 fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, or 400 amino acids. A RL12 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409 or 410.

Optionally, a RL12 fragment can extend further into the N-terminus by 5, 10, 20, or 30 amino acids from the starting residue of the fragment. Optionally, a RL12 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of RL12 or fragment thereof.

RL12 is predicted as a membrane-associated glycoprotein and is a RL11 family member. As described herein, it has been determined that RL12 is a Fc binding protein.

### RL13 Proteins

A RL13 protein can be full length or can omit one or more regions of the protein. Alternatively, fragments of a RL13 protein can be used. RL13 amino acids are numbered according to the full-length RL13 amino acid sequence (CMV RL13 FL) shown in SEQ ID NO: 22, which is 294 amino acids long. Optionally, the RL13 protein can be a RL13 fragment of 10 amino acids or longer. For example, the number of amino acids in the fragment can comprise 10, 15, 20, 30, 40, 50, 60, 70, 80, 90, 100, 125, 150, 175, 200, 225, 250, or 275 amino acids. A RL13 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, or 284 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293 or 294.

Optionally, a RL13 fragment can extend further into the N-terminus by 5, 10, 20, or 30 amino acids from the starting residue of the fragment. Optionally, a RL13 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of RL13 or fragment thereof.

RL13 is a membrane-associated and enveloped glycoprotein and member of the RL11 family. RL13 is highly mutating after in vitro passaging. The wild-type sequence inhibits in vitro virus replication. As described herein, it has been determined that RL13 is a Fc binding protein.

### UL5 Proteins

A UL5 protein can be full length or can omit one or more regions of the protein. Alternatively, fragments of a UL5 protein can be used. UL5 amino acids are numbered according to the full-length UL5 amino acid sequence (CMV UL5 FL) shown in SEQ ID NO: 26, which is 166 amino acids long. Optionally, the UL5 protein can be a UL5 fragment of 10 amino acids or longer. For example, the number of amino acids in the fragment can comprise 10, 15, 20, 30, 40, 50, 60, 70, 80, 90, 100, 125, or 150 amino acids. A UL5 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or 156 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165 or 166.

Optionally, a UL5 fragment can extend further into the N-terminus by 5, 10, 20, or 30 amino acids from the starting residue of the fragment. Optionally, a UL5 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL5 or fragment thereof.

UL5 is a member of the RL11 family and is a predicted membrane protein.

### UL10 Proteins

A UL10 protein can be full length or can omit one or more regions of the protein. Alternatively, fragments of a UL10 protein can be used. Optionally, the UL10 protein can be a UL10 fragment of 10 amino acids or longer. For example, the number of amino acids in the fragment can comprise 10, 15, 20, 30, 40, 50, 60, 70, 80, 90, 100, 125, or 150 amino acids. A UL10 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or 156 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165 or 166.

Optionally, a UL10 fragment can extend further into the N-terminus by 5, 10, 20, or 30 amino acids from the starting residue of the fragment. Optionally, a UL10 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL10 or fragment thereof.

UL10 is a predicted membrane protein. UL10 is proteolytically cleaved in its extracellular domain when expressed in transfected cells.

### UL80.5 Proteins

A UL80.5 protein (also known as pAP) can be full length or can omit one or more regions of the protein. Alternatively, fragments of a UL80.5 protein can be used. UL80.5 amino acids are numbered according to the full-length UL80.5 amino acid sequence (CMV UL80.5 FL) shown in SEQ ID NO: 30, which is 373 amino acids long. Optionally, the UL80.5 protein can be a UL80.5 fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, or 350 amino acids. A UL80.5 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, or 363 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, or 373.

Optionally, a UL80.5 fragment can extend further into the N-terminus by 5, 10, 20, or 30 amino acids from the starting residue of the fragment. Optionally, a UL80.5 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL80.5 or fragment thereof.

UL80.5 is a major capsid scaffold protein. Precursor pAP is cleaved at the C-terminus to yield AP. pAP interacts with MCP (UL80.6).

### UL116 Proteins

A UL116 protein can be full length or can omit one or more regions of the protein. Alternatively, fragments of a UL116 protein can be used. UL116 amino acids are numbered according to the full-length UL116 amino acid sequence (CMV UL116 FL) shown in SEQ ID NO: 34, which is 313 amino acids long. Optionally, the Ul116 protein can be a UL116B fragment of 10 amino acids or longer. For example, the number of amino acids in the fragment can comprise 10, 15, 20, 30, 40, 50, 60, 70, 80, 90, 100, 125, 150, 175, 200, 225, 250, 275, or 300 amino acids. A UL116 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, or 303 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312 or 313.

Optionally, a UL116 fragment can extend further into the N-terminus by 5, 10, 20, or 30 amino acids from the starting residue of the fragment. Optionally, a UL116 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL116 or fragment thereof.

UL116 is a predicted open reading frame and predicted secreted soluble glycoprotein. UL116 protein tracks to the site of virion assembly suggesting it is a viral envelope associated glycoprotein, and potentially interaction with gH and/or gL

### UL119 Proteins

A UL119 protein (also known as gp68) can be full length or can omit one or more regions of the protein. Alternatively, fragments of a UL119 protein can be used. UL119 amino acids are numbered according to the full-length UL119 amino acid sequence (CMV UL119 FL) shown in SEQ ID NO: 38, which is 344 amino acids long. Optionally, the UL119 protein can be a UL119 fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, or 325 amino acids. A UL119 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, or 334 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343 or 344.

Optionally, a UL119 fragment can extend further into the N-terminus by 5, 10, 20, or 30 amino acids from the starting residue of the fragment. Optionally, a UL119 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL119 or fragment thereof.

UL119 (also known as gp68) is a membrane glycoprotein and spliced to UL118. UL119 is a UL119-118 spliced product. UL118, as an individual protein, has never been described. An additional spliced mRNA UL119-UL117 has been found in infected cells, but the protectin has never been described. UL119 is a known Fc binding protein. It has been found on virion and can form complexes with RL11 (See Example 6). It has also been found on the envelope of the virus (See Example 7).

### UL122 Proteins

A UL122 protein (also known as IE2, IE-86) can be full length or can omit one or more regions of the protein. Alternatively, fragments of a UL122 protein can be used. UL122 amino acids are numbered according to the full-length UL122 amino acid sequence (CMV UL122 FL) shown in SEQ ID NO: 42, which is 580 amino acids long. Optionally, the UL122 protein can be a UL122 fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550 or 575 amino acids. A UL122 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569 or 570 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579 or 580.

Optionally, a UL122 fragment can extend further into the N-terminus by 5, 10, 20, or 30 amino acids from the starting residue of the fragment. Optionally, a UL122 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL122 or fragment thereof.

UL122 is an immediate-early transcriptional regulator and has been described as an intermediate-early transcriptional regulator. UL122 is a DNA-binding protein.

### UL132 Proteins

A UL132 protein (also known as gp132) can be full length or can omit one or more regions of the protein. Alternatively, fragments of a UL132 protein can be used. UL132 amino acids are numbered according to the full-length UL132 amino acid sequence (CMV UL132 FL) shown in SEQ ID NO: 46, which is 270 amino acids long. Optionally, the UL132 protein can be a UL132 fragment of 10 amino acids or longer. For example, the number of amino acids in the fragment can comprise 10, 15, 20, 30, 40, 50, 60, 70, 80, 90, 100, 125, 150, 175, 200, 225, or 250 amino acids. A UL132 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, or 260 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269 or 270.

Optionally, a UL132 fragment can extend further into the N-terminus by 5, 10, 20, or 30 amino acids from the starting residue of the fragment. Optionally, a UL132 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL132 or fragment thereof.

UL132 is a membrane protein and envelope glycoprotein and contains a hydrophobic domain. It can internalize from the cell membrane to be inserted into virion.

### UL133 Proteins

A UL133 protein can be full length or can omit one or more regions of the protein. Alternatively, fragments of a UL133 protein can be used. UL133 amino acids are numbered according to the full-length UL133 amino acid sequence (CMV UL133 FL) shown in SEQ ID NO: 50, which is 257 amino acids long. Optionally, the UL133 protein can be a UL133 fragment of 10 amino acids or longer. For example, the number of amino acids in the fragment can comprise 10, 15, 20, 30, 40, 50, 60, 70, 80, 90, 100, 125, 150, 175, 200, 225, or 250 amino acids. A UL133 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, or 247 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256 or 257.

Optionally, a UL133 fragment can extend further into the N-terminus by 5, 10, 20, or 30 amino acids from the starting residue of the fragment. Optionally, a UL133 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL133 or fragment thereof.

### UL138 Proteins

A UL138 protein can be full length or can omit one or more regions of the protein. Alternatively, fragments of a UL138 protein can be used. UL138 amino acids are numbered according to the full-length UL138 amino acid sequence (CMV UL138 FL) shown in SEQ ID NO: 54, which is 169 amino acids long. Optionally, the UL138 protein can be a UL138 fragment of 10 amino acids or longer. For example, the number of amino acids in the fragment can comprise 10, 15, 20, 30, 40, 50, 60, 70, 80, 90, 100, 125, or 150 amino acids. A UL138 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, or 159 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168 or 169.

Optionally, a UL138 fragment can extend further into the N-terminus by 5, 10, 20, or 30 amino acids from the starting residue of the fragment. Optionally, a UL138 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL138 or fragment thereof.

UL138 contains a hydrophobic domain. UL138 predicted one transmembrane. Described as involved in latency, but also required for hematopoietic progenitor cells infection. UL138 is present in Golgi compartment as a membrane protein.

### UL139 Proteins

A UL139 protein can be full length or can omit one or more regions of the protein. Alternatively, fragments of a UL139 protein can be used. Optionally, the UL139 protein can be a UL139 fragment of 10 amino acids or longer. For example, the number of amino acids in the fragment can comprise 10, 15, 20, 30, 40, 50, 60, 70, 80, 90, 100, 125, or 150 amino acids. A UL139 fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, or 159 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168 or 169.

Optionally, a UL139 fragment can extend further into the N-terminus by 5, 10, 20, or 30 amino acids from the starting residue of the fragment. Optionally, a UL139 fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL139 or fragment thereof.

UL139 contains a hydrophobic domain. UL139 predicted as a membrane protein, having at least one transmembrane domain and region of homology with CD24.

### UL148A Proteins

A UL148A protein can be full length or can omit one or more regions of the protein. Alternatively, fragments of a UL148A protein can be used. UL148A amino acids are numbered according to the full-length UL148A amino acid sequence (CMV UL148A FL) shown in SEQ ID NO: 58, which is 80 amino acids long. Optionally, the UL148A protein can be a UL148A fragment of 10 amino acids or longer. For example, the number of amino acids in the fragment can comprise 10, 15, 20, 30, 40, 50, 60, or 70 amino acids. A UL148A fragment can begin at any of residue number: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 and/or terminate at any of residue number 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80.

Optionally, a UL148A fragment can extend further into the N-terminus by 5, 10, 20, or 30 amino acids from the starting residue of the fragment. Optionally, a UL148A fragment can extend further into the C-terminus by 5, 10, 20, or 30 amino acids from the last residue of the fragment.

Typically the CMV protein will have at least 75% identity, at least 80%, at least 85%, at least 90%, at least 95%, at least 97%, at least 98%, or at least 99%, identity to the amino acid sequence of UL148A or fragment thereof.

UL148 is predicted to have one potential transmembrane domain.

### Protein Complexes

Certain of the CMV proteins disclosed herein can associate together to form complexes, and the invention provides for immunogenic complexes comprising two or more human cytomegalovirus (CMV) proteins or fragments thereof. For example, the immunogenic complex may comprise RL11 and UL119 proteins or fragments thereof.

### CMV Antigen Delivery Platforms

The invention provides platforms for delivery of cytomegalovirus (CMV) proteins or fragments to an individual or the cells of an individual. For example, the proteins or fragments can be delivered directly as components of an immunogenic composition, or nucleic acids that encode one or more CMV proteins or fragments can be administered to produce the CMV protein or fragment in vivo. Certain preferred embodiments, such as protein formulations, recombinant nucleic acids (e.g., self replicating RNA, naked or formulated RNA) and alphavirus VRP that contain sequences encoding CMV proteins or fragments are further described herein.

The invention provides platforms for delivery of CMV proteins that may, in some instances, form complexes in vivo. Preferably, these proteins and the complexes they form elicit potent neutralizing antibodies. The immune response produced by delivery of CMV proteins, particularly those that form complexes in vivo (e.g., RL11/UL119), can be superior to the immune response produced using other approaches. For example, a DNA molecule that encodes both RL11 and UL119 of CMV or a mixture of DNA molecules that individually encode RL11 or UL119 can be administered to induce an immune response. In another example, a DNA molecule that encodes both RL13 and UL119 of CMV or a mixture of DNA molecules that individually encode RL13 or UL119 can be administered to induce an immune response. In a further example, a protein complex, such as RL11 and UL119 or RL13 and UL119 (e.g., that is isolated and/or purified) can be administered with or without an adjuvant to induce an immune response.

### Protein Formulations

Immunogenic proteins or fragments thereof used according to the invention will usually be isolated or purified. Thus, they will not be associated with molecules with which they are normally, if applicable, found in nature. Proteins or fragments in the form of a complexes that form normally in vivo, will be associated with other members of the complexes, e.g, RL11 and UL119 or RL13 and UL119.

Proteins, or fragments thereof, will usually be prepared by expression in a recombinant host system. Generally, they (e.g., CMV proteins) are produced by expression of recombinant constructs that encode the proteins in suitable recombinant host cells, although any suitable methods can be used. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), ARPE-19 (ATCC N. CRL-2302) fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding CMV proteins can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

CMV proteins can be purified using any suitable methods. For example, methods for purifying CMV proteins by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the CMV proteins can include a “tag” that facilitates purification, such as an epitope tag or a HIS tag. Such tagged proteins can conveniently be purified, for example from conditioned media, by chelating chromatography or affinity chromatography.

Proteins may include additional sequences in addition to the CMV sequences. For example, a polypeptide may include a sequence to facilitate purification (e.g., a poly-His sequence with or without a linker). Similarly, for expression purposes, the natural leader peptide may be substituted for a different one.

### Alphavirus VRP Platforms

In some embodiments, CMV proteins are delivered using alphavirus replicon particles (VRP). Any nucleotide sequence encoding a CMV protein can be used to produce the protein. As used herein, the term “alphavirus” has its conventional meaning in the art and includes various species such as Venezuelan equine encephalitis virus (VEE; e.g., Trimidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus.

An “alphavirus replicon particle” (VRP) or “replicon particle” is an alphavirus replicon packaged with alphavirus structural proteins.

An “alphavirus replicon” (or “replicon”) is an RNA molecule which can direct its own amplification in vivo in a target cell. The replicon encodes the polymerase(s) which catalyze RNA amplification (nsP1, nsP2, nsP3, nsP4) and contains cis RNA sequences required for replication which are recognized and utilized by the encoded polymerase(s). An alphavirus replicon typically contains the following ordered elements: 5′ viral sequences required in cis for replication, sequences which encode biologically active alphavirus nonstructural proteins (nsP1, nsP2, nsP3, nsP4), 3′ viral sequences required in cis for replication, and a polyadenylate tract. An alphavirus replicon also may contain one or more viral subgenomic “junction region” promoters directing the expression of heterologous nucleotide sequences, which may, in certain embodiments, be modified in order to increase or reduce viral transcription of the subgenomic fragment and heterologous sequence(s) to be expressed. Other control elements can be used, as described below.

Alphavirus replicons encoding one or more CMV proteins are used to produce VRPs. Such alphavirus replicons comprise sequences encoding one or more CMV proteins or fragments thereof. These sequences are operably linked to one or more suitable control element, such as a subgenomic promoter, an IRES (e.g., EMCV, EV71), and a viral 2A site, which can be the same or different. Any one or combination of suitable control elements can be used in any order.

The use of polycistronic vectors is an efficient way of providing nucleic acid sequences that encode two or more CMV proteins in desired relative amounts. In one example, a single subgenomic promoter is operably linked to two sequences encoding two different CMV proteins, and an IRES is positioned between the two coding sequences. In another example, two sequences that encode two different CMV proteins are operably linked to separate promoters. In still another example, the two sequences that encode two different CMV proteins are operably linked to a single promoter. The two sequences that encode two different CMV proteins are linked to each other through a nucleotide sequence encoding a viral 2A site, and thus encode a single amino acid chain that contain the amino acid sequences of both CMV proteins. The viral 2A site in this context is used to generate two CMV proteins from the original polyprotein.

Subgenomic Promoters

Subgenomic promoters, also known as junction region promoters can be used to regulate protein expression. Alphaviral subgenomic promoters regulate expression of alphaviral structural proteins. See Strauss and Strauss, “The alphaviruses: gene expression, replication, and evolution,” Microbiol Rev. 1994 September; 58(3):491-562. A polynucleotide can comprise a subgenomic promoter from any alphavirus. When two or more subgenomic promoters are present, for example in a polycistronic polynucleotide, the promoters can be the same or different. For example, the subgenomic promoter can have the sequence CTCTCTACGGCTAACCTGAATGGA (SEQ ID NO: 1). In certain embodiments, subgenomic promoters can be modified in order to increase or reduce viral transcription of the proteins. See U.S. Pat. No. 6,592,874.

### Internal Ribosomal Entry Site (IRES)

In some embodiments, one or more control elements is an internal ribosomal entry site (IRES). An IRES allows multiple proteins to be made from a single mRNA transcript as ribosomes bind to each IRES and initiate translation in the absence of a 5′-cap, which is normally required to initiate translation. For example, the IRES can be EV71 or EMCV.

Viral 2A Site

The FMDV 2A protein is a short peptide that serves to separate the structural proteins of FMDV from a nonstructural protein (FMDV 2B). Early work on this peptide suggested that it acts as an autocatalytic protease, but other work (e.g., Donnelly et al., (2001), J. Gen. Virol. 82, 1013-1025) suggests that this short sequence and the following single amino acid of FMDV 2B (Gly) acts as a translational stop-start. Regardless of the precise mode of action, the sequence can be inserted between two polypeptides, and effect the production of multiple individual polypeptides from a single open reading frame. FMDV 2A sequences can be inserted between sequences encoding at least two CMV proteins, allowing for their synthesis as part of a single open reading frame. For example, the open reading frame may encode an RL11 protein and a UL119 protein separated by a sequence encoding a viral 2A site. A single mRNA is transcribed then, during the translation step, the RL11 and UL119 peptides are produced separately due to the activity of the viral 2A site. Any suitable viral 2A sequence may be used. Often, a viral 2A site comprises the consensus sequence Asp-Val/Ile-Glu-X-Asn-Pro-Gly-Pro, where X is any amino acid (SEQ ID NO: 2). For example, the Foot and Mouth Disease Virus 2A peptide sequence is DVESNPGP (SEQ ID NO: 3). See Trichas et al., “Use of the viral 2A peptide for bicistronic expression in transgenic mice,” BMC Biol. 2008 Sep. 15; 6:40, and Halpin et al., “Self-processing 2A-polyproteins—a system for co-ordinate expression of multiple proteins in transgenic plants,” Plant J. 1999 February; 17(4):453-9.

In some embodiments an alphavirus replicon is a chimeric replicon, such as a VEE-Sindbis chimeric replicon (VCR) or a VEE strain TC83 replicon (TC83R) or a TC83-Sindbis chimeric replicon (TC83CR). In some embodiments a VCR contains the packaging signal and 3′ UTR from a Sindbis replicon in place of sequences in nsP3 and at the 3′ end of the VEE replicon; see Perri et al., J. Virol. 77, 10394-403, 2003. In some embodiments, a TC83CR contains the packaging signal and 3′ UTR from a Sindbis replicon in place of sequences in nsP3 and at the 3′ end of aVEE strain TC83replicon.

### Producing VRPs

Methods of preparing VRPs are well known in the art. In some embodiments an alphavirus is assembled into a VRP using a packaging cell. An “alphavirus packaging cell” (or “packaging cell”) is a cell that contains one or more alphavirus structural protein expression cassettes and that produces recombinant alphavirus particles after introduction of an alphavirus replicon, eukaryotic layered vector initiation system (e.g., U.S. Pat. No. 5,814,482), or recombinant alphavirus particle. The one or more different alphavirus structural protein cassettes serve as “helpers” by providing the alphavirus structural proteins. An “alphavirus structural protein cassette” is an expression cassette that encodes one or more alphavirus structural proteins and comprises at least one and up to five copies (i.e., 1, 2, 3, 4, or 5) of an alphavirus replicase recognition sequence. Structural protein expression cassettes typically comprise, from 5′ to 3′, a 5′ sequence which initiates transcription of alphavirus RNA, an optional alphavirus subgenomic region promoter, a nucleotide sequence encoding the alphavirus structural protein, a 3′ untranslated region (which also directs RNA transcription), and a polyA tract. See, e.g., WO 2010/019437.

In preferred embodiments, two different alphavirus structural protein cassettes (“split” defective helpers) are used in a packaging cell to minimize recombination events which could produce a replication-competent virus. In some embodiments an alphavirus structural protein cassette encodes the capsid protein (C) but not either of the glycoproteins (E2 and E1). In some embodiments an alphavirus structural protein cassette encodes the capsid protein and either the E1 or E2 glycoproteins (but not both). In some embodiments, an alphavirus structural protein cassette encodes the E2 and E1 glycoproteins but not the capsid protein. In some embodiments an alphavirus structural protein cassette encodes the E1 or E2 glycoprotein (but not both) and not the capsid protein.

In some embodiments, VRPs are produced by the simultaneous introduction of replicons and helper RNAs into cells of various sources. Under these conditions, for example, BHKV cells (1×10⁷) are electroporated at, for example, 220 volts, 1000 μF, 2 manual pulses with 10 μg replicon RNA:6 μg defective helper Cap RNA:10 μg defective helper Gly RNA, alphavirus containing supernatant is collected ˜24 hours later. Replicons and/or helpers can also be introduced in DNA forms which launch suitable RNAs within the transfected cells.

A packaging cell may be a mammalian cell or a non-mammalian cell, such as an insect (e.g., SF9) or avian cell (e.g., a primary chick or duck fibroblast or fibroblast cell line). See U.S. Pat. No. 7,445,924. Avian sources of cells include, but are not limited to, avian embryonic stem cells such as EB66® (VIVALIS); chicken cells, including chicken embryonic stem cells such as EBx® cells, chicken embryonic fibroblasts, and chicken embryonic germ cells; duck cells such as the AGE1.CR and AGE1.CR.pIX cell lines (ProBioGen) which are described, for example, in Vaccine 27:4975-4982 (2009) and WO2005/042728; and geese cells. In some embodiments, a packaging cell is a primary duck fibroblast or duck retinal cell line, such as AGE.CR (PROBIOGEN).

Mammalian sources of cells for simultaneous nucleic acid introduction and/or packaging cells include, but are not limited to, human or non-human primate cells, including PerC6 (PER.C6) cells (CRUCELL N.V.), which are described, for example, in WO 01/38362 and WO 02/40665, as well as deposited under ECACC deposit number 96022940; MRC-5 (ATCC CCL-171); WI-38 (ATCC CCL-75); fetal rhesus lung cells (ATCC CL-160); human embryonic kidney cells (e.g., 293 cells, typically transformed by sheared adenovirus type 5 DNA); VERO cells from monkey kidneys); cells of horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001); cat, and rodent (e.g., hamster cells such as BHK21-F, HKCC cells, or Chinese hamster ovary (CHO) cells), and may be obtained from a wide variety of developmental stages, including for example, adult, neonatal, fetal, and embryo.

In some embodiments a packaging cell is stably transformed with one or more structural protein expression cassette(s). Structural protein expression cassettes can be introduced into cells using standard recombinant DNA techniques, including transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection. Structural protein expression cassettes typically are introduced into a host cell as DNA molecules, but can also be introduced as in vitro-transcribed RNA. Each expression cassette can be introduced separately or substantially simultaneously.

In some embodiments, stable alphavirus packaging cell lines are used to produce recombinant alphavirus particles. These are alphavirus-permissive cells comprising DNA cassettes expressing the defective helper RNA stably integrated into their genomes. See Polo et al., Proc. Natl. Acad. Sci. USA 96, 4598-603, 1999. The helper RNAs are constitutively expressed but the alphavirus structural proteins are not, because the genes are under the control of an alphavirus subgenomic promoter (Polo et al., 1999). Upon introduction of an alphavirus replicon into the genome of a packaging cell by transfection or VRP infection, replicase enzymes are produced and trigger expression of the capsid and glycoprotein genes on the helper RNAs, and output VRPs are produced. Introduction of the replicon can be accomplished by a variety of methods, including both transfection and infection with a seed stock of alphavirus replicon particles. The packaging cell is then incubated under conditions and for a time sufficient to produce packaged alphavirus replicon particles in the culture supernatant.

Thus, packaging cells allow VRPs to act as self-propagating viruses. This technology allows VRPs to be produced in much the same manner, and using the same equipment, as that used for live attenuated vaccines or other viral vectors that have producer cell lines available, such as replication-incompetent adenovirus vectors grown in cells expressing the adenovirus E1A and E1B genes.

In some embodiments, a two-step process is used: the first step comprises producing a seed stock of alphavirus replicon particles by transfecting a packaging cell with a plasmid DNA-based replicon. A much larger stock of replicon particles is then produced in a second step, by infecting a fresh culture of packaging cells with the seed stock. This infection can be performed using various multiplicities of infection (MOI), including a MOI=0.00001, 0.00005, 0.0001, 0.0005, 0.001, 0.005, 0.01, 0.05, 0.1, 0.5, 1.0, 3, 5, 10 or 20. In some embodiments infection is performed at a low MOI (e.g., less than 1). Over time, replicon particles can be harvested from packaging cells infected with the seed stock. In some embodiments, replicon particles can then be passaged in yet larger cultures of naive packaging cells by repeated low-multiplicity infection, resulting in commercial scale preparations with the same high titer.

### Nucleic Acid Delivery Systems

Recombinant nucleic acid molecule that encode one or more CMV proteins or fragments can be administered to induce production of the encoded CMV proteins or fragments and an immune response thereto. The recombinant nucleic acid can be based on any desired nucleic acid such as DNA (e.g., plasmid or viral DNA) or RNA, preferably self replicating RNA, and can be monocystronic or polycistronic. Any suitable DNA or RNA can be used as the nucleic acid vector that carries the open reading frames that encode CMV proteins or fragments thereof. Suitable nucleic acid vectors have the capacity to carry and drive expression of one or more CMV proteins or fragments. Such nucleic acid vectors are known in the art and include, for example, plasmids, DNA obtained from DNA viruses such as vaccinia virus vectors (e.g., NYVAC, see U.S. Pat. No. 5,494,807), and poxvirus vectors (e.g., ALVAC canarypox vector, Sanofi Pasteur), and RNA obtained from suitable RNA viruses such as alphavirus. If desired, the recombinant nucleic acid molecule can be modified, e.g., contain modified nucleobases and or linkages as described further herein.

Recombinant nucleic acid molecules that are polycistronic provide the advantage of delivering sequences that encode two or more CMV proteins to a cell, and for example driving the expression of the CMV proteins at sufficient levels to result in the formation of a protein complex containing the two or more CMV proteins in vivo. Using this approach, two or more encoded CMV proteins that form a complex can be expressed at sufficient intracellular levels for the formation of CMV protein complexes (e.g., RL11/UL119 or RL13/UL119). For example, the encoded CMV proteins or fragments thereof can be expressed at substantially the same level, or if desired, at different levels by selecting appropriate expression control sequences (e.g., promoters, IRES, 2A site etc.). This is a significantly more efficient way to produce protein complexes in vivo than by co-delivering two or more individual DNA molecules that encode different CMV to the same cell, which can be inefficient and highly variable. See, e.g., WO 2004/076645.

The self-replicating RNA molecules of the invention are based on the genomic RNA of RNA viruses, but lack the genes encoding one or more structural proteins. The self-replicating RNA molecules are capable of being translated to produce non-structural proteins of the RNA virus and CMV proteins encoded by the self-replicating RNA.

The self-replicating RNA generally contains at least one or more genes selected from the group consisting of viral replicase, viral proteases, viral helicases and other nonstructural viral proteins, and also comprise 5′- and 3′-end cis-active replication sequences, and a heterologous sequences that encodes one or more desired CMV proteins. A subgenomic promoter that directs expression of the heterologous sequence(s) can be included in the self-replicating RNA. If desired, a heterologous sequence may be fused in frame to other coding regions in the self-replicating RNA and/or may be under the control of an internal ribosome entry site (IRES).

Self-replicating RNA molecules of the invention can be designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alpha virus, such as Sinbis virus (SIN), Semliki forest virus and Venezuelan equine encephalitis virus (VEE), one or more genes encoding viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to a vertebrate cell even without any proteins, lead to the production of multiple daughter RNAs by transcription from itself (or from an antisense copy of itself). The self-replicating RNA can be directly translated after delivery to a cell, and this translation provides a RNA-dependent RNA polymerase which then produces transcripts from the delivered RNA. Thus the delivered RNA leads to the production of multiple daughter RNAs. These transcripts are antisense relative to the delivered RNA and may be translated themselves to provide in situ expression of encoded CMV protein, or may be transcribed to provide further transcripts with the same sense as the delivered RNA which are translated to provide in situ expression of the encoded CMV protein(s).

One suitable system for achieving self-replication is to use an alphavirus-based RNA replicon, such as an alphavirus replicon as described herein. These + stranded replicons are translated after delivery to a cell to give off a replicase (or replicase-transcriptase). The replicase is translated as a polyprotein which auto cleaves to provide a replication complex which creates genomic − strand copies of the + strand delivered RNA. These − strand transcripts can themselves be transcribed to give further copies of the + stranded parent RNA and also to give a subgenomic transcript which encodes two or more CMV proteins. Translation of the subgenomic transcript thus leads to in situ expression of the CMV protein(s) by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) one or more CMV proteins or fragments thereof. The polymerase can be an alphavirus replicase e.g. comprising alphavirus protein nsP4.

Whereas natural alphavirus genomes encode structural virion proteins in addition to the non structural replicase polyprotein, it is preferred that an alphavirus based self-replicating RNA molecule of the invention does not encode all alphavirus structural proteins. Thus the self replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self replicating RNAs of the invention and their place is taken by gene(s) encoding the desired gene product (CMV protein or fragment thereof), such that the subgenomic transcript encodes the desired gene product rather than the structural alphavirus virion proteins.

Thus a self-replicating RNA molecule useful with the invention has one or more sequences that encode CMV proteins or fragments thereof. The sequences encoding the CMV proteins or fragments can be in any desired orientation, and can be operably linked to the same or separate promoters. If desired, the sequences encoding the CMV proteins or fragments can be part of a single open reading frame. In some embodiments the RNA may have one or more additional (downstream) sequences or open reading frames e.g. that encode other additional CMV proteins or fragments thereof. A self-replicating RNA molecule can have a 5′ sequence which is compatible with the encoded replicase.

In one aspect, the self-replicating RNA molecule is derived from or based on an alphavirus, such as an alphavirus replicon as defined herein. In other aspects, the self-replicating RNA molecule is derived from or based on a virus other than an alphavirus, preferably, a positive-stranded RNA virus, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro virus (ATCC VR-66; ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The self-replicating RNA molecules of the invention can contain one or more modified nucleotides and therefore have improved stability and be resistant to degradation and clearance in vivo, and other advantages. Without wishing to be bound by any particular theory, it is believed that self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduces activation of the innate immune system and subsequent undesired consequences (e.g., inflammation at injection site, irritation at injection site, pain, and the like). It is also believed that the RNA molecules produced as a result of self-replication are recognized as foreign nucleic acids by the cytoplasmic immune receptors. Thus, self-replicating RNA molecules that contain modified nucleotides provide for efficient amplification of the RNA in a host cell and expression of CMV proteins, as well as adjuvant effects.

As used herein, “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). If desired, a self replicating RNA molecule can contain chemical modifications in or on the sugar moiety of the nucleoside (e.g., ribose, deoxyribose, modified ribose, modified deoxyribose, six-membered sugar analog, or open-chain sugar analog), or the phosphate.

The self-replicating RNA molecules can contain at least one modified nucleotide, that preferably is not part of the 5′ cap. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties.

Suitable modifications that can be included in the self-replicating RNA molecules are known in the art and described, for example, in WO2011/005799. The skilled addressee is directed to the disclosure of WO2011/005799 at paragraphs 66-72, which is incorporated herein by reference.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses.

Suitable synthetic methods can be used alone, or in combination with one or more other methods (e.g., recombinant DNA or RNA technology), to produce a self-replicating RNA molecule that contain one or more modified nucleotides. Suitable methods for de novo synthesis are well-known in the art and can be adapted for particular applications. Exemplary methods include, for example, chemical synthesis using suitable protecting groups such as CEM (Masuda et al., (2007) Nucleic Acids Symposium Series 51:3-4), the β-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedron Lett 27:4055-8; Gaffney B L et al. (1988) Tetrahedron Lett 29:2619-22). These chemistries can be performed or adapted for use with automated nucleic acid synthesizers that are commercially available. Additional suitable synthetic methods are disclosed in Uhlmann et al. (1990) Chem Rev 90:544-84, and Goodchild J (1990) Bioconjugate Chem 1: 165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation and expression of nucleic acid sequences are known and conventional in the art. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

The presence and/or quantity of one or more modified nucleotides in a self-replicating RNA molecule can be determined using any suitable method. For example, a self-replicating RNA can be digested to monophosphates (e.g., using nuclease P1) and dephosphorylated (e.g., using a suitable phosphatase such as CIAP), and the resulting nucleosides analyzed by reversed phase HPLC (e.g., using a YMC Pack ODS-AQ column (5 micron, 4.6×250 mm) and eluted using a gradient, 30% B (0-5 min) to 100% B (5-13 min) and at 100% B (13-40) min, flow Rate (0.7 ml/min), UV detection (wavelength: 260 nm), column temperature (30° C.). Buffer A (20 mM acetic acid—ammonium acetate pH 3.5), buffer B (20 mM acetic acid—ammonium acetate pH 3.5/methanol [90/10])).

The self-replicating RNA may be associated with a delivery system. The self-replicating RNA may be administered with or without an adjuvant.

### RNA Delivery Systems

The self-replicating RNA described herein are suitable for delivery in a variety of modalities, such as naked RNA delivery or in combination with lipids, polymers or other compounds that facilitate entry into the cells. Self-replicating RNA molecules can be introduced into target cells or subjects using any suitable technique, e.g., by direct injection, microinjection, electroporation, lipofection, biolystics, and the like. The self-replicating RNA molecule may also be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues (SEQ ID NO: 4)), which is itself coupled to an integrin receptor-binding moiety (e.g., a cyclic peptide having the sequence Arg-Gly-Asp).

The self-replicating RNA molecules can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

The self-replicating RNA can be delivered as naked RNA (e.g. merely as an aqueous solution of RNA) but, to enhance entry into cells and also subsequent intercellular effects, the self-replicating RNA is preferably administered in combination with a delivery system, such as a particulate or emulsion delivery system. A large number of delivery systems are well known to those of skill in the art. Such delivery systems include, for example liposome-based delivery (Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Felgner et al. (1987) Proc. Natl. Acad. Sci. USA 84: 7413-7414), as well as use of viral vectors (e.g., adenoviral (see, e.g., Berns et al. (1995) Ann. NY Acad. Sci. 772: 95-104; Ali et al. (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5): 1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl. Acad. Sci. USA, 81:6466-6470; McLaughlin et al. (1988) and Samulski et al. (1989) J. Virol., 63:03822-3828), and the like.

Three particularly useful delivery systems are (i) liposomes, (ii) non-toxic and biodegradable polymer microparticles, and (iii) cationic submicron oil-in-water emulsions.

Such delivery systems are known in the art and described, for example, in WO2011/005799. The skilled addressee is directed to paragraphs 90-126 of WO2011/005799, which is incorporated herein by reference.

Catheters or like devices may be used to deliver the self-replicating RNA molecules of the invention, as naked RNA or in combination with a delivery system, into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

The present invention includes the use of suitable delivery systems, such as liposomes, polymer microparticles or submicron emulsion microparticles with encapsulated or adsorbed self-replicating RNA, to deliver a self-replicating RNA molecule that encodes two or more CMV proteins, for example, to elicit an immune response alone, or in combination with another macromolecule. The invention includes liposomes, microparticles and submicron emulsions with adsorbed and/or encapsulated self-replicating RNA molecules, and combinations thereof.

The self-replicating RNA molecules associated with liposomes and submicron emulsion microparticles can be effectively delivered to a host cell, and can induce an immune response to the protein encoded by the self-replicating RNA.

Polycistronic self replicating RNA molecules that encode CMV proteins, and VRPs produced using polycistronic alphavirus replicons, can be used to form CMV protein complexes in a cell. Complexes include, but are not limited to, RL11/UL119 and RL13/UL119.

In some embodiments combinations of VRPs or VRPs that contain sequences encoding two or more CMV proteins or fragments are delivered to a cell. Combinations include, but are not limited to:

### 1. a RL11/UL119 VRP;

### 2. a RL11 VRP and a UL119 VRP;

### 3. a RL13/UL119 VRP; and

### 4. a RL13 VRP and a UL119 VRP.

In some embodiments combinations of self-replicating RNA molecules or self replicating RNA molecules that encode two or more CMV proteins or fragments are delivered to a cell. Combinations include, but are not limited to:

1. self-replicating RNA molecule encoding RL11 and UL119;
2. a self-replicating RNA molecule encoding RL11 and a self-replicating RNA molecule encoding UL119;
3. self-replicating RNA molecule encoding RL13 and UL119; and
4. a self-replicating RNA molecule encoding RL13 and a self-replicating RNA molecule encoding UL119.

### Methods and Uses

In some embodiments, proteins, DNA molecules, self-replicating RNA molecules or VRPs are administered to an individual to stimulate an immune response. In such embodiments, proteins, DNA molecules, self-replicating RNA molecules or VRPs typically are present in a composition which may comprise a pharmaceutically acceptable carrier and, optionally, an adjuvant. See, e.g., U.S. Pat. No. 6,299,884; U.S. Pat. No. 7,641,911; U.S. Pat. No. 7,306,805; and US 2007/0207090.

The immune response can comprise a humoral immune response, a cell-mediated immune response, or both. In some embodiments an immune response is induced against each delivered CMV protein. A cell-mediated immune response can comprise a Helper T-cell (Tₕ) response, a CD8+ cytotoxic T-cell (CTL) response, or both. In some embodiments the immune response comprises a humoral immune response, and the antibodies are neutralizing antibodies. Neutralizing antibodies block viral infection of cells. CMV infects epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing; i.e., an antibody generated against an administered composition neutralizes a CMV virus of a strain other than the strain used in the composition.

A useful measure of antibody potency in the art is “50% neutralization titer.” To determine 50% neutralizing titer, serum from immunized animals is diluted to assess how dilute serum can be yet retain the ability to block entry of 50% of viruses into cells. For example, a titer of 700 means that serum retained the ability to neutralize 50% of virus after being diluted 700-fold. Thus, higher titers indicate more potent neutralizing antibody responses. In some embodiments, this titer is in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50% neutralization titer range can have an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 6500. “About” means plus or minus 10% of the recited value. Neutralization titer can be measured as described in the specific examples, below.

An immune response can be stimulated by administering proteins, DNA molecules, self-replicating RNA molecules or VRPs to an individual, typically a mammal, including a human. In some embodiments the immune response induced is a protective immune response, i.e., the response reduces the risk or severity of CMV infection. Stimulating a protective immune response is particularly desirable in some populations particularly at risk from CMV infection and disease. For example, at-risk populations include solid organ transplant (SOT) patients, bone marrow transplant patients, and hematopoietic stem cell transplant (HSCT) patients. VRPs can be administered to a transplant donor pre-transplant, or a transplant recipient pre- and/or post-transplant. Because vertical transmission from mother to child is a common source of infecting infants, administering VRPs to a woman who is pregnant or can become pregnant is particularly useful.

Any suitable route of administration can be used. For example, a composition can be administered intra-muscularly, intra-peritoneally, sub-cutaneously, or trans-dermally. Some embodiments will be administered through an intra-mucosal route such as intra-orally, intra-nasally, intra-vaginally, and intra-rectally. Compositions can be administered according to any suitable schedule.

In another aspect, nucleic acids encoding two or more CMV proteins selected from the group consisting of RL10, RL11, RL12, RL13, UL5, UL80.5, UL116, UL119, UL122, UL132, UL133, UL138, UL139, and UL148A are delivered to a cell, and the cell is maintained under conditions suitable for expression of said first CMV protein and said second CMV protein, to form a CMV protein complex. The cell may be in vivo. Preferably, the cell is an epithelial cell, an endothelial cell, or a fibroblast. In a preferred aspect, nucleic acids encoding RL11 and UL119 are delivered to a cell, and the cell is maintained under conditions suitable for expression of RL11 CMV protein and UL119 CMV protein, to form a RL11/UL119 CMV protein complex. In another preferred aspect, nucleic acids encoding RL13 and UL119 are delivered to a cell, and the cell is maintained under conditions suitable for expression of RL13 CMV protein and UL119 CMV protein, to form a RL13/UL119 CMV protein complex.

In another aspect, nucleic acids encoding a first one or more CMV proteins selected from the group consisting of RL10, RL11, RL12, RL13, UL5, UL80.5, UL116, UL119, UL122, UL132, UL133, UL138, UL139, and UL148A are delivered to a cell, and a second one or more CMV proteins selected form the group consisting of gB, gH, gL; gO; gM, gN; UL128, UL130, UL131 are delivered to a cell, and the cell is maintained under conditions suitable for expression of said first CMV protein and said second CMV protein to form a CMV protein complex. The cell may be in vivo. Preferably, the cell is an epithelial cell, an endothelial cell, or a fibroblast.

In another aspect, an immunogenic composition or immunogenic complex of the invention is used to contact a cell, as a method of inhibiting CMV entry into the cell.

All patents, patent applications, and references cited in this disclosure, including nucleotide and amino acid sequences referred to by accession number, are expressly incorporated herein by reference. The above disclosure is a general description. A more complete understanding can be obtained by reference to the following specific examples, which are provided for purposes of illustration only.

### Example 1

### Bioinformatics

### A. Material and Methods

### Genome Sequences

Ten HCMV genome sequences, representing 8 different strains were analyzed. They were directly derived from completed genome sequences stored in the GenBank database: NC_{—}001347 (AD169), AY315197 (Towne), AC146905 (Toledo), AC146907 (FIX), AC146904 (PH), AC146906 (TR), AC146999 (AD169-BAC), AC146851 (Towne-BAC), NC_{—}00623 (Merlin) and EF999921 (TB40/E-BAC4).

The human cytomegalovirus strains are conventionally classified in high-passage and low-passage strains based on the number of passages in human fibroblasts (HFs) in culture before they were cloned using bacterial artificial chromosomes (BAC) and then sequenced.

The NUCmer algorithm from MUMmer 3.21 package (Kurtz S. et al., 2004; http://mummer.sourceforge.net) was used to align AD169 and Merlin genomes. MUMmer uses a suffix-tree approach to find maximal unique matches (MUM). NUCmer (NUCleotide MUMmer), first runs MUMmer to find all exact matches longer than a specified length (option—1 20). Then, the matches are clustered in preparation for extending them. Two matches are joined into the same cluster if they are separated by no more than 90 (—g option) nucleotides. Then from each cluster, the maximum-length collinear chain of matches is extracted and processed further if the combined length of its matches is at least 65 nucleotides. The chain matches are then extended using an implementation of the Smith-Waterman dynamic programming algorithm (Smith and Waterman 1981), which is applied to the regions between the exact matches and also to the boundaries of the chains, which may be extended outward.

A sequence comparison using BLASTN (Altschul S. F. et al., 1990) was performed to map homologies and rearrangements between the two genomes and the results were visualized using the Artemis Comparison Tool (ACT) release 8 from Sanger Institute (Carver T. J. et al., 2005; http://www.sanger.ac.uk/Software/ACT).

### Annotation and Homologs Detection

Coding sequences were generated from all analyzed genomes with the exception of Merlin by the getorf program from the EMBOSS suite (Rice P. et al, 2000). A minimum coding potential of 20 amino acids (—minsize 60 option) and standard code with alternative initiation codons (—table 1 option) were expected.

The potential splicing patterns were analyzed using TIGR GeneSplicer (Pertea M. et al., 2001) prediction tool, a statistical method that predicts splice sites by integrating multiple sources of evidence. It reaches very good performance in terms of accuracy and computational efficacy.

The sequence similarity searching FASTAv35.4.3 algorithm (Pearson W R and Lipman D J, 1988) was used to compare Merlin proteins with all ORFs with BLOSUM50 as substitution matrix and expectation value upper limit for score of 1E-5. The output was parsed by ad hoc developed scripts based on BioPerl 1.6 code libraries (Stajich J E et al., 2002; BioPerl http://www.bioperl.org/) to extract only matches with at least 70% amino acid sequence identity between query and hit over more than 75% of “overlap.” The overlap is defined as the ratio between the matching hit sequence length and the query sequence length. The ORFs outperforming these thresholds were considered putative coding sequences (CDSs). The CDSs from each genome and Merlin protein were aligned to determine the conservation level using CLUSTALW (Thompson J D et al., 1994) with a progressive alignment strategy that is sufficient for highly similar proteins.

### Protein Topology Predictions

Phobius (Käll et al., 2004; http://phobius.sbc.su.se/) was used for prediction of transmembrane topology and signal peptides from the amino acid sequence of identified proteins. This predictor program is able to discriminate between the hydrophobic regions of a transmembrane helix and those of a signal peptide. Their high similarity often leads to misinterpretations between the two types of predictions. The predictor is based on a hidden Markov model (HMM) that models the different sequence regions of a signal peptide and the different regions of a transmembrane protein in a series of interconnected states. Compared to TMHMM and SignalP, errors coming from cross-prediction were reduced substantially by Phobius. False classifications of signal peptides are 3.9% and false classifications of transmembrane helices are 7.7%.

### Pattern-Matching Extraction

PatMatch (Yan T. et al., 2005) available at (ftp://ftp.arabidopsis.org/home/tair/Software/Patmatch) was used to identify ER retention/retrieval motifs and Rb binding domains. It enables searches for short sequences by a powerful and flexible pattern syntax based on regular expressions. It also supports both mismatches and wildcards in a single pattern by implementing a nondeterministic-reverse grep (NR-grep).

### Glycosylation Sites Predictions

NetngLYC 1.0 (Gupta R. et al., 2004) and NetOGlyc 3.1 (Julenius K. et al., 2004) were used to identify potential post-translational modification sites. NetNGlyc algorithm (http://www.cbs.dtu.dk/services/NetNGlyc/) is based on artificial neural networks trained on the surrounding sequence context to discriminate between acceptor and non-acceptor sites. In a cross-validated performance, the networks could identify 86% of the glycosylated and 61% of the non-glycosylated sequences, with an overall accuracy of 76%. NetOGlyc algorithm (http://www.cbs.dtu.dk/services/NetOGlyc/) uses a neural network approach for predicting the location for mucin-type glycosylation sites, trained on the O-GLYCBASE db, a total of 86 mammalian proteins experimentally investigated for in vivo O-GalNAc sites. Moreover, it uses the structural information of 12 glycosylated structures obtained from the Protein Data Bank. The NetOGlyc final prediction arises from a combination of networks, the best overall network used as input amino acid composition, averaged surface accessibility predictions together with substitution matrix profile encoding of the sequence. To improve prediction on isolated (single) sites, networks were trained on isolated sites only. The prediction method correctly predicts 76% of the glycosylated residues and 93% of the non-glycosylated residues. Apart from characterizing individual proteins, both methods can rapidly scan complete proteomes.

### B. Results

### Detection of Genomic Rearrangements and Variability

The complete published DNA sequences of AD169 and Merlin (accession numbers NC_{—}001347 and NC_{—}006273) were analyzed to compare the repeated sequences and rearrangements between laboratory strains and clinical isolates. They were chosen as representatives of high passage and low passage strains, respectively. The two genomes were aligned using MUMmer to identify duplications and inversions. The genome comparison was performed using BLASTN to locate the rearrangement regions and visualized by ACT. The analysis showed that the AD169 genome is 230,290 base pairs in size, while Merlin is 5,356 base pairs longer and the overall sequence identity between the two genomes is 93.3%. The two genomes are collinear, except for a large genomic rearrangement occurring in the laboratory strain at the right side of the major unique region UL. When compared to Merlin, AD169 lacks completely a segment of 15.3 kbp (here named A), spanning from 179,543 to 194,852 nt coordinates in Merlin, that is partially replaced by a sequence of 10.5 kbp (179155-189697 nt coordinates in AD169, named B). This sequence is an inverted duplication of the region laying between 1.4 k and 10 kbp both in the AD169 and Merlin genomes. Downstream of this variability region (around 19.5 kbp in the Merlin genome), there is a segment of 2 kbp (C segment) that is duplicated and inverted at the extreme right boundary of the TRS region in both strains. Briefly, the AD169 strain genome has two duplications, B=10 kbp and C=2 kpb. Only C is present in the Merlin strain.

Genomic alignment allowed for observation of the lack of colinearity among the two genomes. The regions of variability were identified along TRL region until the junction with UL (˜18 kb), around 94 and 107 kb in the UL, at the junction IRS/US and US/TRS (˜197 kb and 233 kb respectively). The coordinates refer to Merlin sequence.

Moreover, the RefSeq annotation of AD169 and Merlin indicate the canonical genomic organization (TRL-UL-IRL-IRS-US-TRS) as reported in Table 1.

TABLE 1

| | Position in AD169 genome | | Position in Merlin genome | |
|---|---|---|---|---|
| Region | Start-stop (nn length) | | Start-stop (nn length) | |
| a | 1-578 | (578) | 1-578 | (578) |
| TRL | 1-11247 | (11247) | 1-1324 | (1324) |
| UL | 11248-179152 | (167905) | 1325-194343 | (193019) |
| IRL | 179153-190400 | (11248) | 194344-195667 | (1324) |
| a | 189823-190400 | (578) | 195090-195667 | (578) |
| IRS | 189823-192345 | (2523) | 195090-197626 | (2537) |
| US | 192346-227766 | (35421) | 197627-233108 | (35428) |
| TRS | 227767-230290 | (2524) | 233109-235643 | (2538) |
| a | 229663-230290 | (628) | 235068-235646 | (579) |

There are notable differences in the TRL, UL and IRL regions lengths between the two genomes (a difference of 9923 nn for TRL, 25114 nn for UL, 9924 nn for IRL).

After the comparative analysis, each genomic region in both the analyzed strains was able to be re-located (Table 2). The Terminal Repeated Long (TRL) region contains repeats that are between 1.4 k and 10 kbp, as previously described. They are organized as follows:

TABLE 2
Genomic regions organization in AD169 and Merlin arising
from our analysis.The canonical genomic organization is listed
with the corresponding coordinates in AD169 genome.
The Merlin genomic coordinates resulting from the
comparative study in highlighted in bold, in the third column.

| | Position in AD169 genome | | Position in Merlin genome | |
|---|---|---|---|---|
| Region | Start-stop (nn length) | | Start-stop (nn length) | |
| a | 1-578 | (578) | 1-578 | (578) |
| TRL | 1-11247 | (11247) | 1-11785 | (11785) |
| UL | 11248-179152 | (167905) | 11786-179540 | (167754) |
| IRL | 179153-190400 | (11248) | 179541-195667 | (16126) |
| a | 189823-190400 | (578) | 195090-195667 | (578) |
| IRS | 189823-192345 | (2523) | 195090-197626 | (2537) |
| US | 192346-227766 | (35421) | 197627-233108 | (35428) |
| TRS | 227767-230290 | (2524) | 233109-235643 | (2538) |
| a | 229663-230290 | (628) | 235068-235646 | (579) |

The comparison analysis highlights sequence variability patterns that emphasize large divergences between the most studied laboratory strain AD169 and the wild type Merlin.

### Conservation of Protein Coding Genes

The Merlin genome was selected as a reference because it is the only one considered as a wild-type strain containing ORF092 (Dolan et al., 2004). Merlin is part of the RefSeq database, and has been recognized containing a total of 165 genes, about 12 of which are spliced. Their genomic sequences were analyzed with GeneSplicer, a computational method for splice site prediction. The predictions were compared with the Merlin genes annotation. All acceptor and donor sites for the 12 spliced gene products were confirmed.

The coding content of the remaining 9 genomes was re-evaluated by determining the set of putative coding sequences (CDSs) that are conserved in most of the analyzed genomes.

First, all start-to-stop open reading frames (ORFs) with a very short sequence coding potential of 20 amino acids within each of the genomes were identified using the getorf program from the EMBOSS suite (Rice P. et al., 2000). Similar previous studies used to filter the minimum size standard of 80 amino acids or more. Evidence suggests that this choice lead to the exclusion of known gene products because CMV CDSs length varies between 22 amino acids of CMV006 and 2241 amino acids of CMV110. Moreover, the analysis of the proteins associated with HCMV virions proposed by Varnum and coworkers (2004) raised the possibility that the virus encodes some very small polypeptides, so a decision was made to extend the analysis to very short sequences. The presence of the rarely used alternate start codons (GUG, CUG and UUG), reported by Brondke et al. in 2007, was accounted for in the identification of all ORFs.

A database containing the ORFs derived from each genome was built and Merlin proteins were searched against it to identify their homologues in each genome. The sequence similarity searching FASTAv35 algorithm (Pearson W R and Lipman D J 1988) was used since it has resulted in more accurate detection of matches' boundaries in comparison with BLAST (Basic Local Alignment Search Tool; Altschul et al., 1990) algorithm.

All ORFs filtered by a series of cutoff parameters were considered homologues to Merlin proteins and putative CDSs for the remaining genomes. These criteria impose an e-value lower than 1e-5 and a sequence identity higher than 70% over more than 75% of the full length Merlin query protein (“overlap”). The overlap is defined as the ratio between the matching sequence length and the query (Merlin protein) sequence length. In such a way, the best hit in each of the 9 genomes was identified as a homolog of each Merlin protein.

Most of the recognized ORFs (93%) were highly conserved, while the others exhibited high variability among strains. The least conserved CDSs are along the regions of variability already highlighted by the previous genomic analysis. Following the order of the conventional map they are ORF082-83, ORF003-7, ORF009-12 belonging to TRL/UL until roughly 18 kb and ORF087-88 at 107 kb. Further proteins, ORF014, ORF020-21, ORF026, ORF039, ORF053-54, ORF048, ORF057 presented low similarity level due to shorter regions of variability or point mutations. Over the 165 proteins annotated for the Merlin genome, 154 are well conserved in all of the six clinical isolates.

The major rearrangement highlighted by previously described genomic comparison between AD169 and Merlin implies important differences at the protein coding level. The present analysis confirmed most of the sequence variation already described in the literature (Cha et al., 1996; Prichard et al., 2001). The same considerations can be extended to all analyzed strains (four laboratory and six low passage strains). As previously described for AD169, the AD169-BAC genome also lacks a sequence coding for 19 proteins (ORF044-55, ORF056A-B-C-D, ORF057) in low passage strains. A similar, but smaller, sequence is missing from Towne and Towne-BAC coding for 15 proteins (ORF044-7, ORF052-5, ORF056A-B-C-D, ORF057).

The low passage strains, like Merlin, do not have duplicated proteins.

Due to the insertion of BAC sequences, part of US region of seven genomes (Murphy et al., 2003; see above Materials and Methods for details) is disrupted, so only a part of the sequences could be compared. Several gene sequences were confirmed as missing. Moreover, even though PH, FIX, and TB40E genomes should lack only the ORF058-ORF060 region for BAC insertion, an anticipated deletion of ORF113 and ORF112 genes that was observed in previous studies (Sinzger C. et al., 2008; Murphy E. et al., 2003) was also confirmed. Moreover, in the same region, the coding of ORF066 in Toledo and CMV060 in TR appeared to be altered.

Frameshifts in ORF004 (RL13) (in AD169, Towne) and ORF094A in AD169 (Skaletskaya et al., 2001) were confirmed.

ORF048, ORF052 and ORF053 are hypervariable (Brondly, Davison 2008), so all sequence publicly available at GenBank databases were collected and multiple alignments were performed to better characterize specific patterns of variability. This allowed for a frameshift mutation for ORF004 (RL13) and ORF094A in PH and for ORF012 in Toledo to be marked. For ORF012, a single nucleotide mutation that introduces an anticipated stop codon in PH was found.

For PH, Toledo and TR strains, a set of putative CDSs (7) with high sequence conservation levels that have not been previously reported were identified (Table 3). This revealed errors in several annotated genomes.

TABLE 3
Putative novel CDS identified in PH, Toledo and TR strains.
The amino acid sequence identity percentages compared with
the Merlin homologs are indicated in parentheses.

| Strain | Gene name (aa identity %) |
|---|---|
| PH | ORF056a (UL148a) (96%), ORF056c (100%), ORF080a (95%) |
| TOLEDO | ORF056c (100%), ORF056d (95%), ORF080a (97%) |
| TR | ORF056c (100%) |

### Potentially Surface-Exposed Proteins

All HCMV proteins were evaluated by computational methods to infer their localization and allow for selection of potentially surface exposed proteins. Phobius (Kall et al., 2004) was used to predict transmembrane domains and signal peptides starting from the amino acid sequence. 94 proteins of interest were identified (see Table 4 for the complete list). Evidence for the presence of a signal peptide was found in 75 proteins and evidence of transmembrane domain was found in 48 proteins. Twenty-nine of the proteins exhibited both a signal peptide and a transmembrane domain.

Most of the antigens described in the literature for vaccine formulation lay in this set, for example, the members of envelope glycoproteins complexes gcI(gB), gcII (gM/gN) and gcIII (gH/gL/gO) and gH/gL/ORF092/ORF093/ORF094 (Compton et al., 2004; Ryckman B J et al., 2008) that are essential for the entry in several types of host cells and cell tropism (Wang D. et al., 2005). Structural, early and late antigens and HCMV-encoded immunomodulators (pp 28, pp 50, ORF058, ORF059, ORF060 and ORF019) (Elkington et al., 2003) were also found.

Interestingly, 79 of the identified proteins could be suggested as new putative antigens. Moreover, by crossing these analyses and the results of protein conservation levels (previous paragraph) 45 proteins were elicited showing high conservation levels (more than 95% AA conservation) among all low passage strains and then ideal candidates for a cross-protective vaccine.

### The Glycoproteins of Cytomegalovirus

A prediction of potential glycosylations sites among the selected proteins was carried out. Both O-glycolsilation and N-glycosilation site predictions were performed by NetOGlyc 3.1 and NetNGlyc 1.0 (see Materials and methods above for detailed information). Since only extracellular domains may be glycosylated in transmembrane proteins, the results from this analysis were crossed with topological analysis. All of the predicted sites in N-terminal signal peptides and potential transmembrane domains were ruled out.

The analysis predicts that, from the 94 proteins, 77 proteins could have N-glycosylated sites and 71 proteins could have O-glycosylated sites (see Table 4). A confidence score was assigned to all predicted Asn modification.

Potential glycosylation sites were predicted for all gene products (48) already annotated as glycosylated. Although ORF015, ORF016, ORF017, ORF029, ORF030, ORF032, ORF034, ORF037 (UL116), ORF039, ORF040, ORF058, ORF070, ORF071, ORF072, ORF073, ORF077 and ORF080 are not annotated as glycosylated, many potential modification sites were recognized, some of them confirm previous work (Rigoutsos et al., 2002). The prediction analysis identified further potential glycoproteins: ORF024, ORF031, ORF041 (UL122), ORF045, ORF046, ORF047 (UL138), ORF049, ORF053 ORF057.

The results of the topological analysis allowed the selection of 94 proteins over the total 165. Putative signal peptide (SP) and/or the hydrophobic domain (TM) are listed in the third column. The results of glycosylation predictions are also shown. The number of potential N-glycosylation sites is indicated in the third column with the statistical confidence of the prediction: (+++) and (++) for high specificity predictions; (+) for good specificity. Fourth column show how many potential O-glycosylation prediction were predicted for each protein. All data refer to Merlin protein sequences.

TABLE 4
Proteins predicted as secreted or membrane
associated and their potential glycosylated sites.

| | | Topo- | N. of | N. of |
|---|---|---|---|---|
| | | logical | N-glyc. | O-glyc. |
| Proteins | Characterization | prediction | sites (η) | sites |
| ORF001 | Envelope glycoprotein | 1SP; | 3x (+) | 2 |
| | | 1TM | | |
| ORF002 | Membrane-associated | 1SP; | 4x (++) | 2 |
| | IgG Fc-binding | 1TM | | |
| | glycoprotein; ORF002 | | | |
| | family member | | | |
| ORF003 | Membrane-associated | 2TM | 1x (+++), | 29 |
| | glycoprotein; ORF002 | | 8x (++), | |
| | family member | | 8x (+) | |
| ORF004 | Membrane-associated | 1SP; | 2x (+++), | 24 |
| | glycoprotein; ORF002 | 1TM | 3x (++), | |
| | family member | | 6x (+) | |
| ORF005 | Membrane-associated | 1SP; | 2x (+++), | 0 |
| | glycoprotein; ORF002 | 1TM | 2x (++), | |
| | family member | | 4x (+) | |
| ORF006 | Potential membrane | 1TM | none | 0 |
| | protein | | | |
| ORF007 | Envelope glycoprotein; | 1SP | 2x (+++), | 1 |
| | ORF002 family member | | 4x (++), | |
| | | | 3x (+) | |
| ORF008 | Potential membrane | 1TM | none | 5 |
| | glycoprotein; ORF002 | | | |
| | family member | | | |
| ORF009 | Glycoprotein; ORF002 | 1TM | 1x (+++), | 10 |
| | family member | | 2x (++), | |
| | | | 6x (+) | |
| ORF010 | Membrane-associated | 2TM | 3x (+++), | 12 |
| | glycoprotein; ORF002 | | 3x (++), | |
| | family member | | 3x (+) | |
| ORF011 | Membrane-associated | 1TM | 2x (++) | 10 |
| | glycoprotein; ORF002 | | | |
| | family member | | | |
| ORF012 | Membrane-associated | 1SP; | 3x (++), | 2 |
| | glycoprotein; ORF002 | 1TM | 2x (+) | |
| | family member | | | |
| ORF013 | Glycoprotein; ORF002 | 1TM | 1x (+++), | 5 |
| | family member | | 2x (++), | |
| | | | 1x (+) | |
| ORF014 | Membrane-associated | 2TM | 2x (+++), | 33 |
| | glycoprotein; ORF002 | | 2x (++), | |
| | family member | | 2x (+) | |
| ORF015 | Potentially secreted | 1SP | 2x (+) | 6 |
| ORF016 | Membrane-associated | 1SP; | 1x (++) | 1 |
| | protein; ORF016 | 1TM | | |
| | family member | | | |
| ORF017 | Potential membrane | 1TM | 1x (++) | 2 |
| | protein | | | |
| ORF018 | Membrane-associated | 1SP; | 1x (+++), | 0 |
| | glycoprotein; binds to | 1TM | 4x (++), | |
| | MHC class 1-related | | 3x (+) | |
| | molecules | | | |
| ORF019 | Membrane glycoprotein; | 1SP; | 2x (+++), | 3 |
| | similar to MHC class I; | 1TM | 3x (++), | |
| | ORF019 family member | | 8x (+) | |
| ORF020 | Membrane-associated | 2TM | 6x (++), | 3 |
| | glycoprotein; similar to | | 7x (+) | |
| | T cell receptor gamma | | | |
| | chain | | | |
| ORF021 | Secreted glycoprotein; | 1SP | 1x (++) | 16 |
| | spliced | | | |
| ORF022 | Tegument protein; | 1SP | none | 1 |
| | ORF104 family member | | | |
| ORF023 | ORF104 family member | 1SP | none | 1 |
| ORF024 | Herpesvirus-specific gp | 1SP | 3x (+) | 10 |
| ORF025 | Envelope glycoprotein; | 7TM | 1x (++), | 1 |
| | G-protein coupled | | 5x (+) | |
| | receptor; GPCR family | | | |
| | member; spliced | | | |
| ORF026 | Full length is envelope | 2TM | 2x (+++), | 1 |
| | glycoprotein; viral | | 8x (++), | |
| | mitochondrial inhibitor | | 5x (+) | |
| | of apoptosis (vMIA) | | | |
| | located in N-terminal | | | |
| | domain specified by | | | |
| | first exon; spliced | | | |
| ORF027 | Membrane-associated | 1SP | (+) | 2 |
| | glycoprotein; contains | | | |
| | HLA-E-binding | | | |
| | peptide and upregulates | | | |
| | HLA-E | | | |
| ORF028 | Envelope glycoprotein | 1TM | (+) | 2 |
| ORF029 | Predicted membrane | 1TM | (+) | 0 |
| | protein | | | |
| ORF030 | Tegument protein | 1TM | 2x (++) | 7 |
| ORF031 | Membrane-associated | 1TM | 2x (++) | 11 |
| | protein involved in | | | |
| | egress of capsids from | | | |
| | nucleus | | | |
| ORF086 | Envelope glycoprotein | 1SP; | 3x (+++), | 4 |
| | | 1TM | 6x (++), | |
| | | | 7x (+) | |
| ORF087 | Envelope glycoprotein | 1SP; | 1x (+) | 31 |
| | | 1TM | | |
| ORF088 | Envelope glycoprotein | 1SP; | 1x (+++), | 6 |
| | | 1TM | 3x (++), | |
| | | | 7x (+) | |
| ORF089 | Envelope glycoprotein | 1SP; | 1x (+++), | 4 |
| | | 1TM | 2x (++), | |
| | | | 3x (+) | |
| ORF032 | Envelope protein; | 7TM | (+++) | 7 |
| | putative G-protein | | | |
| | coupled receptor; GPCR | | | |
| | family member | | | |
| ORF033 | Major capsid scaffold | 1SP | (+) | 29 |
| | protein | | | |
| ORF034 | DNA packaging protein; | 2TM | 1x (+++), | 1 |
| | putative ATPase subunit | | 1x (+) | |
| | of terminase; spliced | | | |
| ORF090 | Envelope glycoprotein | 8TM | 2x (++), | 0 |
| | | | 3x (+) | |
| ORF035 | Component of DNA | 2TM | 1x (+++), | 8 |
| | helicase-primase | | 2x (++), | |
| | complex | | 7x (+) | |
| ORF036 | Interleukin-10 | 1SP | (++) | 4 |
| ORF091 | Envelope glycoprotein | 1SP | (+++) | 1 |
| ORF037 | Predicted membrane | 1SP | 4x (++), | 63 |
| | protein | | 7x (+) | |
| ORF038 | Glycoprotein | 1SP; | 2x (+++), | 26 |
| | | 1TM | 3x (++), | |
| | | | 7x (+) | |
| ORF039 | Predicted type I | 1SP; | 6x (++), | 0 |
| | membrane protein | 2TM | 1x (+) | |
| ORF040 | Membrane protein | 1SP; | (+) | 0 |
| | | 1TM | | |
| ORF041 | Immediate-early | 1TM | (++) | 35 |
| | transcriptional | | | |
| | regulator; spliced | | | |
| ORF042 | Membrane glycoprotein | 1SP; | 1x (++), | 23 |
| | | 1TM | 1x (+) | |
| ORF092 | Envelope protein | 1SP | N/A | N/A |
| ORF093 | Envelope glycoprotein | 1SP | 3x (+) | 4 |
| ORF094 | Envelope protein | 1SP | (+++) | 0 |
| ORF043 | Envelope glycoprotein | 1SP; | 1x (++), | 17/15 |
| | | 1TM | 2x (+) | |
| ORF044 | Predicted membrane | 2TM | none | 14 |
| | glycoprotein | | | |
| ORF045 | Predicted secreted | 1SP | (++) | 43 |
| ORF046 | Predicted membrane | 1TM | none | 10 |
| | protein | | | |
| ORF047 | Golgi-localized type I | 1TM | none | 9 |
| | membrane | | | |
| ORF048 | Membrane-associated | 1SP; | 1x (++), | 35 |
| | glycoprotein | 1TM | 1x (+) | |
| ORF049 | Predicted membrane | 1TM | 1x (++), | 9 |
| | protein | | 1x (+) | |
| ORF050 | Membrane-associated | 1SP; | 3x (+) | 2 |
| | glycoprotein; ORF016 | 1TM | | |
| | family member | | | |
| ORF051 | Membrane-associated | 1SP; | 1x (+++), | 11/11 |
| | glycoprotein; similar to | 1TM | 6x (++), | |
| | MHC class I; ORF019 | | 10x (+) | |
| | family member | | | |
| ORF052 | Membrane-associated | 1SP; | 1x (+++), | 0 |
| | glycoprotein | 1TM | 1x (++), | |
| | similar to TNFR | | 3x (+) | |
| ORF053 | Alpha-chemokine; | 1SP | 3x (++) | 0 |
| | ORF053 family | | | |
| | member | | | |
| ORF054 | Putative alpha- | 1TM | none | 0 |
| | chemokine; ORF053 | | | |
| | family member | | | |
| ORF055 | Membrane-associated | 1SP; | none | 0 |
| | contains hydrophobic | 1TM | | |
| | domain | | | |
| ORF056 | Membrane-associated | 1SP; | 1x (++), | 1 |
| | protein | 1TM | 2x (+) | |
| ORF056A | Predicted membrane | 1TM | none | 1 |
| | protein | | | |
| ORF056B | Predicted membrane | 1TM | (++) | 2 |
| | protein | | | |
| ORF056C | Predicted membrane | 2TM | none | 1 |
| | protein | | | |
| ORF056D | Predicted membrane | 1TM | (−) | 5 |
| | protein | | | |
| ORF057 | Transmembrane | 1SP | (+) | 17 |
| | protein | | | |
| ORF058 | Degradation of MHC-I | 1TM | 1x (++), | 0 |
| | (and possibly MHC-II) | | 1x (+) | |
| ORF059 | Membrane-associated | 1SP; | 1x (++) | 0 |
| | immediate-early | 1TM | | |
| | glycoprotein; US2 | | | |
| | family member | | | |
| ORF060 | Glycoprotein; ORF060 | 1SP; | 1x (++) | 1 |
| | family member | 1TM | | |
| ORF061 | Membrane-associated | 1SP; | 1x (++), | 1/0 |
| | glycoprotein; ORF060 | 1TM | 1x (+) | |
| | family member | | | |
| ORF062 | Membrane-associated | 1SP; | 1x (+++) | 0 |
| | glycoprotein; ORF060 | 1TM | | |
| | family member | | | |
| ORF063 | Membrane-associated | 1SP; | 1x (++), | 0 |
| | glycoprotein; role in | 1TM | 1x (+) | |
| | cell-to-cell spread in | | | |
| | epithelial cells; | | | |
| | ORF060 family | | | |
| | member | | | |
| ORF064 | Membrane-associated | 1SP; | 1x (++), | 1 |
| | glycoprotein; ORF060 | 1TM | 1x (+) | |
| | family member | | | |
| ORF065 | Membrane-associated | 1SP; | 1x (+++) | 3 |
| | glycoprotein; ORF060 | 1TM | | |
| | family member | | | |
| ORF066 | Membrane-associated | 7TM | none | 4 |
| | multiply hydrophobic | | | |
| | protein; ORF066 | | | |
| | family member | | | |
| ORF067 | Membrane-associated | 7TM | none | 0 |
| | multiply hydrophobic | | | |
| | protein; ORF066 | | | |
| | family member | | | |
| ORF068 | Membrane-associated | 7TM | 1x (+++) | 12 |
| | multiply hydrophobic | | | |
| | protein; ORF066 | | | |
| | family member | | | |
| ORF069 | Membrane-associated | 7TM | none | 0 |
| | multiply hydrophobic | | | |
| | protein; ORF066 | | | |
| | family member | | | |
| ORF070 | Membrane-associated | 7TM | (+) | 9 |
| | multiply hydrophobic | | | |
| | protein; ORF066 | | | |
| | family member | | | |
| ORF071 | Membrane-associated | 7TM | (+) | 6 |
| | multiply hydrophobic | | | |
| | protein; ORF066 | | | |
| | family member | | | |
| ORF072 | Membrane-associated | 7TM | (++) | 3 |
| | multiply hydrophobic | | | |
| | protein; ORF066 | | | |
| | family member | | | |
| ORF072 | Membrane-associated | 7TM | none | 0 |
| | multiply hydrophobic | | | |
| | protein; ORF066 | | | |
| | family member | | | |
| ORF073 | Membrane-associated | 7TM | 1x (++), | 5 |
| | multiply hydrophobic | | 2x (+) | |
| | protein; ORF066 | | | |
| | family member | | | |
| ORF074 | Membrane-associated | 7TM | none | 6 |
| | multiply hydrophobic | | | |
| | protein; ORF066 | | | |
| | family member | | | |
| ORF076 | Envelope glycoprotein; | 7TM | 5x (++), | 3 |
| | G-protein coupled | | 1x (+) | |
| | receptor; GPCR family | | | |
| | member | | | |
| ORF077 | G-protein coupled | 7TM | (++) | 9 |
| | receptor; GPCR family | | | |
| | member | | | |
| ORF078 | Predicted membrane | 1SP; | 1x (+++), | 13 |
| | glycoprotein | 2TM | 1x (++), | |
| | | | 1x (+) | |
| ORF079 | Predicted membrane | 1SP; | 1x (++), | 8 |
| | glycoprotein | 1TM | 2x (+) | |
| ORF080 | Predicted secreted | 1SP | 2x (++), | 0 |
| | protein | | 1x (+) | |
| ORF080A | Predicted membrane | 1TM | none | 0 |
| | protein | | | |

### Example 2

### Reverse Vaccinology Approach

### Gene Synthesis

Nucleic acids that encoded the amino acid sequences derived from the bioinformatics analysis described in Example 1 were synthesized. Synthesis was requested with optimized codons for Homo sapiens usages, and attachment of a 5′ untranslated region containing AscI and SalI site for future cloning convenience, as well as a Kozak sequence for efficient protein translation (5′-GCTAGCGGCGCGCCGTCGACGCCACC) (SEQ ID NO: 5). Synthesized genes were inserted into the NheI (5′) and BamHI (3′) sites of pcDNAmyc His version A (−) (Invitrogen) were requested. These pcDNA clones were used for transfection into cultured cell lines for protein expression in vitro.

### Production of Alphavirus Replicon Plasmid and Particles

The alphavirus replicon plasmids were prepared by digesting pcDNA clones first with BamHI and AflII to remove the c-myc and hexahistidine (SEQ ID NO: 6) encoding sequence in the pcDNAmyc His version A (−) vector. After blunt-end formation of E. coli DNA polymerase in vitro, the plasmid DNA was re-circularized with T4 DNa polymerase. The re-circularized DNA was transformed into commercial E. coli competent cells (DH5α® from Invitrogen or XL-1 Blue® from Stratagene) using procedures provided by the manufacturer, to obtain sufficient amount of plasmid DNA from the shorter pcDNA clone. The plasmids were further digested with AflII. After blunt-end formation by E. coli DNA polymerase in vitro, the DNA was digested with AscI. The DNA fragment containing a CMV gene sequence was isolated by agarose gel electrophoresis and inserted in the VCR-chim2.1 vector (AscI and blunt-ended NotI sites). The resulting DNA was again transformed into E. coli competent cells. The VCR clones were used for production of VRP.

The alphavirus replicon particles were prepared as follows:

In Vitro Transcription of Replicon RNA and Defective Helper RNA

VRP plasmid, DH(defective helper)-Gly, and DH-Cap plasmid were linearized independently by digestion with PmeI restriction enzyme. The linearized DNA were purified using Qiaquick® DNA purification column kit (Qiagen). A half microgram of the purified DNA was submitted to a commercially available in vitro transcription kit (e.g. mMESSAGE mMACHINE from Ambion). Yielded RNA were further treated with DNase and purified using reagent included in the kit.

Triple RNA Electroporation

BHK-V cells were cultivated in high glucose DMEM medium supplemented with 10% FBS in T-225 or T175 flasks in an incubator at 37° C. with 5% CO₂. Cells were detached with trypsin. After 1.5 minutes at 37° C., trypsin was inactivated by addition of FBS containing fresh DMEM medium. Detached cells were collected in centrifugation tubes and pelleted by centrifugation at 4° C., for 5 minutes, at 1500 rpm using an Eppendorf tabletop centrifuge (5810R). Cell pellets were rinsed with RNase-free PBS three times. Cells were resuspended in cold Optimem (LifeTechnologies) at a concentration of 2×10⁷/ml.

Replicon RNA (10 μg), DH-Gly (6 μg) and DH-Cap RNA (10 μg) were placed in an electroporation cuvette (e.g. BioRad 165-2088 or Eppendorf #4307-002-022) on ice. Five hundred μl of cell suspension in Optimem were added to the cuvette. The cuvette was placed in an electroporator (GenePulser XCell from BioRad) using the following conditions (Exponential Decay protocol: 220V, 1000 μF infinite resistance, 4 mm gap). The electric pulses were given twice manually. The pulsed cells were transferred to a T75 flask containing prewarmed DMEM (14.5 ml) supplemented with 5% FBS. After 24 hours of cultivation at 37° C. in a CO₂ incubator, the culture supernatant was collected and centrifuged at 3000 rpm (Eppendorf 5180R) for 15 minutes at 4° C. to remove cell debris. The supernatant was transferred to an ultracentrifuge tube (Beckman #344058). One ml of 20% sucrose in PBS was underlayed beneath the supernatant. One ml of 50% sucrose in PBS was underlayed beneath the 20% sucrose layer.

VRP Concentration on Sucrose Cushion

The samples on the sucrose cushion were centrifuged for 2 hours at 30,000 rpm in a SW32Ti rotor at 4° C. The majority of the media part was aspirated to discard, leaving approximately 0.5 ml. The remaining material was added with 10 ml of buffered MEM (2× Eagle's MEM Lonza #12-668E, 20 mM HEPES, without FBS) and transferred to an Amicon Ultra-15 (Millipore #UFC910024) concentrator, followed by centrifugation at 4° C. for 30 to 45 minutes at 2,500 rpm till the solution is concentrated to 0.75 ml. The flow-through was discarded and 12 ml of buffered 1× Minimal Essential Medium were added to the solution above the filter. The centrifugation was repeated to reduce the volume to 1 ml. The concentrated VRP were divided into several aliquots and stored at −80° C.

### VRP Immunization

Female mice Balb/c (BALB/cAnNCrl), were purchased at the age of 6 weeks from Charles River Laboratories, Calco, Italy. Replicon particles were diluted to appropriate concentrations in PBS. Mice were immunized 2-3 times intra-muscularly in the tibialis anterior muscle with a total of 10⁵-10⁶ infectious units in 50 μl of PBS/mouse with 3 weeks of interval between administrations. Serum was prepared for serological analyses from the blood of immunized mice after 2-3 weeks of immunization.

### Transfections

The plasmid DNA were transfected to cultured cells (HEK 293T). Cell lysates were prepared from the transfectants to perform immunoblot using anti-histidine antibody as well as mouse sera from the immunized mice (Table 5).

The plasmid DNA were transfected to cultured cells (HEK 293T). Transfected cells were permeabilized and immunofluorescent assays were performed using anti-myc antibody, as well as mouse sera from the immunized mice (Table 5).

The plasmid DNA were transfected to cultured cells (HEK 293T). Cell lysates were prepared from the transfectants to perform immunoblot using CytoGam®, a commercial products that contain high titer of anti-CMV antibodies derived from CMV infected individuals. Antibodies against the following proteins were found in Cytogam®: RL10, RL12, RL13, UL5, UL7, UL11, UL33, UL40, UL41A, UL80.5, UL116, UL119, UL122, UL132, UL133, UL136, UL139, UL141, UL148A, US20, and US27 (Table 5).

TABLE 5
Results of transfections of 293T cells (“6His” disclosed as SEQ ID NO: 6)

| | Expressed in 293T | Antibodies in immune | Antibodies in |
|---|---|---|---|
| | cells (by 6His-or | mouse sera detected | CytoGam |
| Gene | myc-tag) | by immunoblot | (293T cells) |
| RL10 | +++ | +/− | ++ |
| RL11 | +++ | + | − |
| RL12 | − | maybe | + |
| RL13 | ++ | − | +++ |
| UL1 | +++ | ++ | − |
| UL2 | ++ | − | − |
| UL4 | +++ | − | − |
| UL5 | ++ | | − |
| UL6 | + | − | − |
| UL7 | +++ | − | + |
| UL8 | + | − | − |
| UL9 | +++ | − | − |
| UL10 | ++ | | − |
| UL11 | ++ | | − |
| UL13 | ++ | − | − |
| UL14 | ++ | maybe | − |
| UL15A | + | may not be | − |
| UL16 | +++ | − | − |
| UL18 | +++ | − | − |
| UL20 | ++ | | − |
| UL22A | − | | − |
| UL24 | ++ | − | − |
| UL29 | +++ | | − |
| UL31 | ++ | − | − |
| UL33 | + | − | − |
| UL37 | +++ | − | − |
| UL40 | ++ | − | ++ |
| UL41A | +++ | − | − |
| UL42 | ++ | + | − |
| UL148C | + | − | − |
| UL148D | ++ | − | − |
| UL150 | + (MF) | | − |
| US2 | + | | − |
| US3 | − | | − |
| US6 | ++ | | − |
| US7 | ++ | + | − |
| US8 | ++ | + | − |
| US9 | ++ | | − |
| US10 | + | − | − |
| US11 | ++ | +/− | − |
| US12 | ++ | | − |
| US13 | + | | − |
| US14 | + | − | − |
| US15 | − | − | − |
| US16 | ++ | − | − |
| US17 | ++ | − | − |
| UL47 | ++ | − | − |
| US18 | − | − | − |
| US19 | + | − | − |
| US20 | − | − | + |
| US21 | ++ | | − |
| US27 | ++ | − | ++ |
| US28 | + | | − |
| US29 | + | − | + |
| US30 | ++ | | − |
| US34 | + | − | − |
| US34A | − | − | − |
| | Expressed in 293T | Antibodies in immune | Antibodies in |
| | cells (by 6His-or | mouse sera detected | CytoGam |
| Gene | myc-tag, | by immunoblot | (293T cells) |
| UL50 | − | | − |
| UL78 | ++ | | − |
| UL80.5 | +++ | ++ | +++ |
| UL89 | − | − | − |
| UL105 | + | − | − |
| UL111A | +++ | − | − |
| UL116 | +++ | − | − |
| UL119 | ++ | − | +++ |
| UL120 | ++ | maybe | − |
| UL121 | ++ | − | − |
| UL122 | ++ | | +++ |
| UL124 | ++ | + | − |
| UL132 | +++ | − | +++ |
| UL133 | ++ | + | ++ |
| UL135 | ++ | − | − |
| UL136 | ++ | − | ++ |
| UL138 (Cam) | + | ++ | − |
| UL138 (Sie) | + | + | − |
| UL139 | ++ | − | +++ |
| UL140 | + | − | − |
| UL141 | + | maybe | − |
| UL142 | − | − | − |
| UL144 | + | − | − |
| UL146 | ++ | − | − |
| UL147 | ++ | | − |
| UL147A | − | − | − |
| UL148 | + | | |
| UL148A | +++ | maybe | − |
| UL148B | ++ | − | − |

(+++) = very likely glycosylated
(++) = likely glycosylated
(+) = could be glycosylated

### Confocal Microscopy

The plasmid DNA were transfected to cultured cells (ARPE-19 and MRC-5). Cells were permeabilized and confocal microscopy analysis was performed using anti-c-myc antibody, as well as CytoGam® or Cytotect® to study subcellular localization (Table 6).

TABLE 6

| Gene | Intracellular localization in ARPE-19 and MRC-5 |
|---|---|
| RL10 | Endoplasmic Reticulum |
| RL11 | Golgi, Trans-golgi network, Early endosomes |
| RL12 | Endoplasmic Reticulum, Golgi, |
| | Trans-golgi network, Early endosomes |
| RL13 | Golgi, Trans-golgi network, Early endosomes |
| UL5 | Golgi |
| UL80.5 | Nuclear |
| UL116 | Endoplasmi Reticulum |
| UL119 | Golgi, Trans-golgi network, Early endosomes |
| UL122 | Nuclear |
| UL132 | Trans-golgi network |
| UL133 | Trans-golgi network |

### Neutralization Assays

CMV neutralizing antibodies in mouse sera were measured using a microneutralization assay (IE1 Focus Assay), stained 48 hours post-infection. A 50 μl volume of an adequate virus dilution (TB40 EGFP, previously titered to have nearly 100 positive cells/well) in growth medium (D-MEM/F12 1:1 containing 10% heat-inactivated FBS and penicillin/streptomycin glutamine mix, plus sodium pyruvate) was added to an equal volume of serial dilutions of heat-inactivated test serum in the same medium containing 10% guinea pig complement, in 96-well tissue culture plates. The serum/CMV/complement mixture was incubated at 37° C. for one hour, then 100 μl of an ARPE-19 suspension (4×10⁵ cells/ml) was added and plates were cultured for 2 days at 37° C. in 5% CO2. Wells were fixed with 10% buffered formalin (100 μl/well) for 1 hour at room temperature (RT), washed three times with PBS 1% Triton-X100 (300 μl/well) and then permeabilized for 1 hour at RT with saponin buffer (PBS, 2% FBS, 0.5% saponin). After removal of permeabilizing solution wells were reacted with anti-IE1 monoclonal antibody conjugated with Alexa-488 (Millipore, MAB 810×). Plates were washed three times with PBS 1% Triton-X100 (300 μl/well) and the number of cells expressing IE1 was determined by fluorescence microscopy. The percent reduction in the number of IE1-positive cells compared to control wells was calculated for each sample and the serum was considered neutralizing if capable of at least 50% reduction of infectivity.

### Example 3

### Identification of Novel FcBP Coded by HCMV

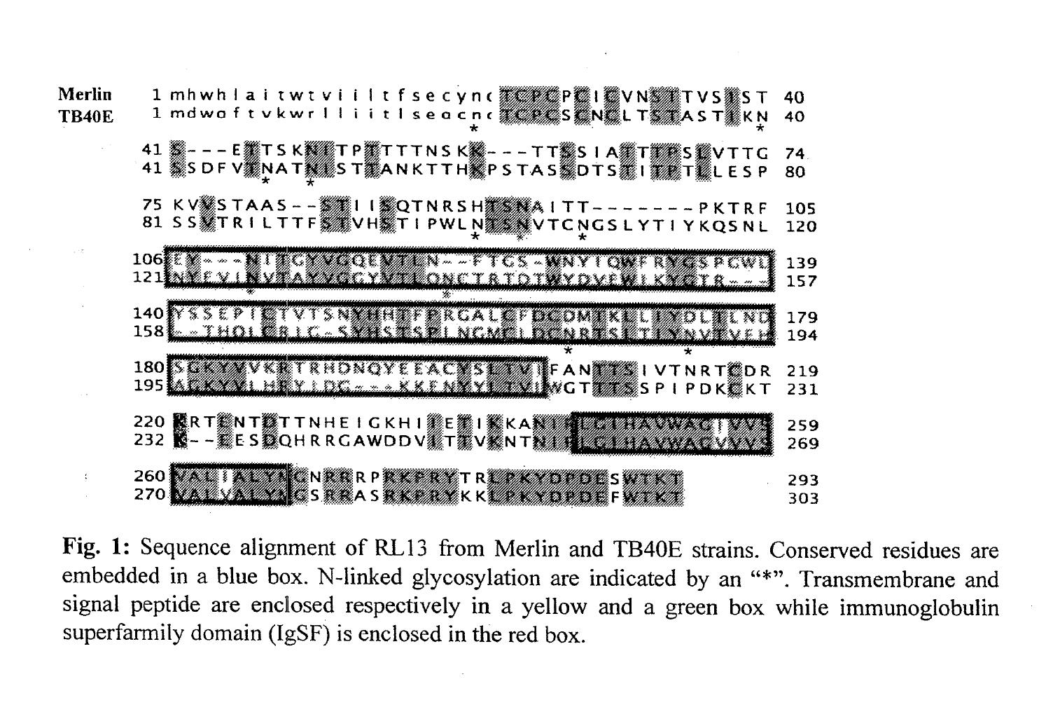

RL13 is known to be a transmembrane glycoprotein that belongs to the RL11 subfamily. Like UL119 it contains an Immunoglobulin super family (IgSF) domain and has been reported to have a high glycosylation status with both N- and O-linked glycans (FIG. 1). Due to these characteristics, the ability of RL13 to bind hFc was tested.

Far-Western blot analyses using human non-immune immunoglobulin (non-immune hIgG) was performed. As control gpRL10 was used, a protein belonging to the RL11 subfamily exhibiting one transmembrane domain (ref.) but lacking the IgG-like domain.

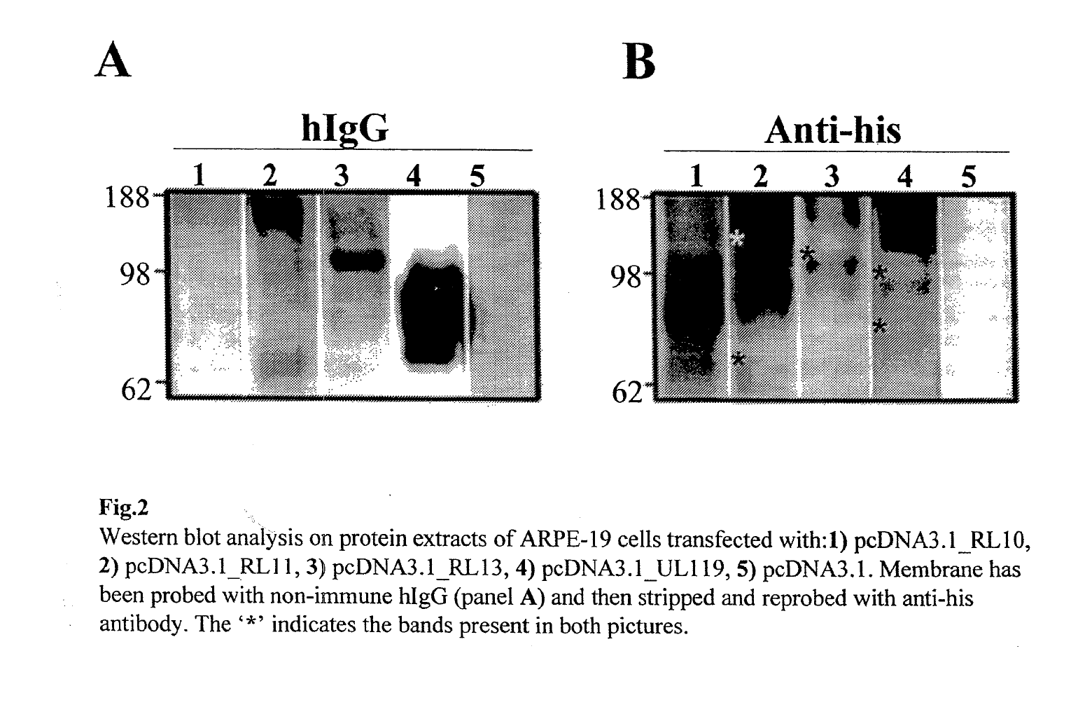

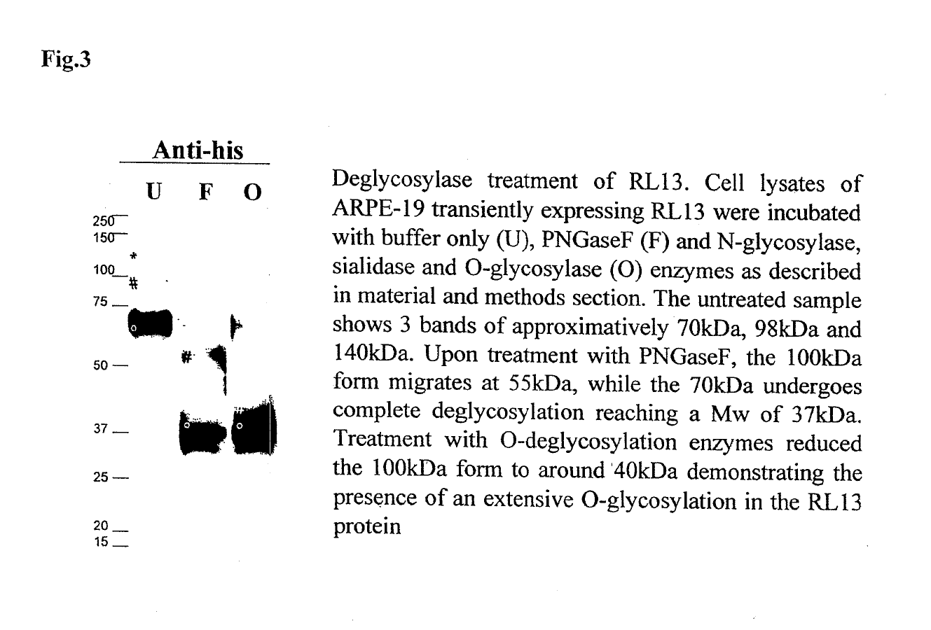

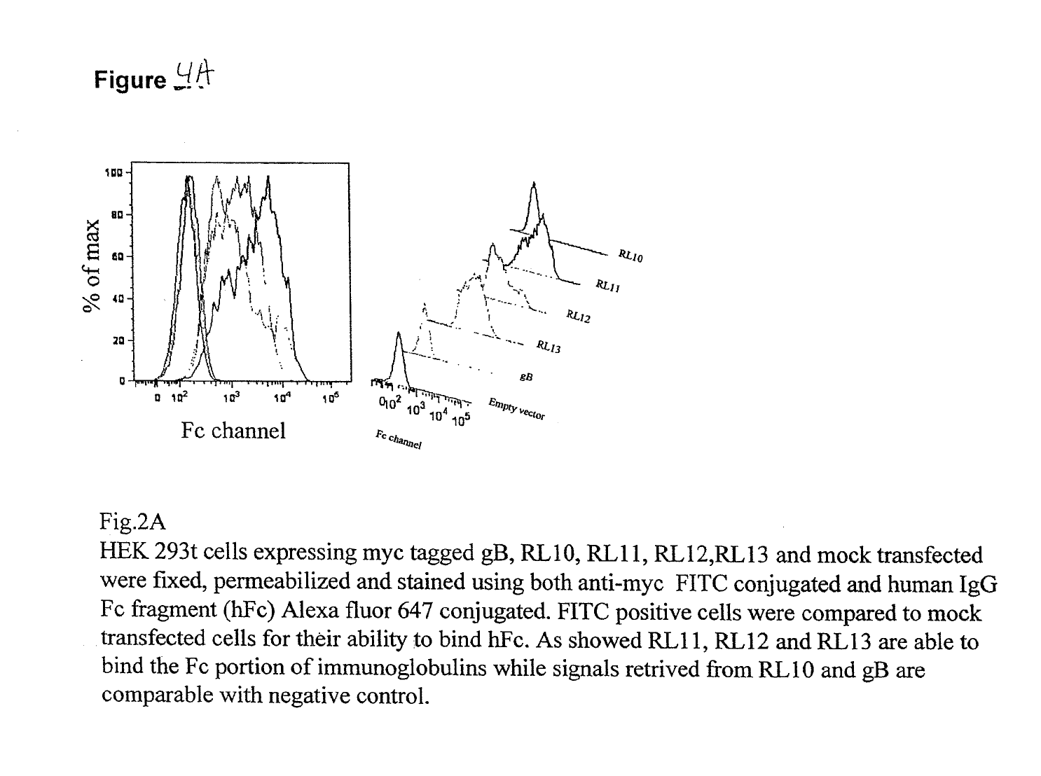

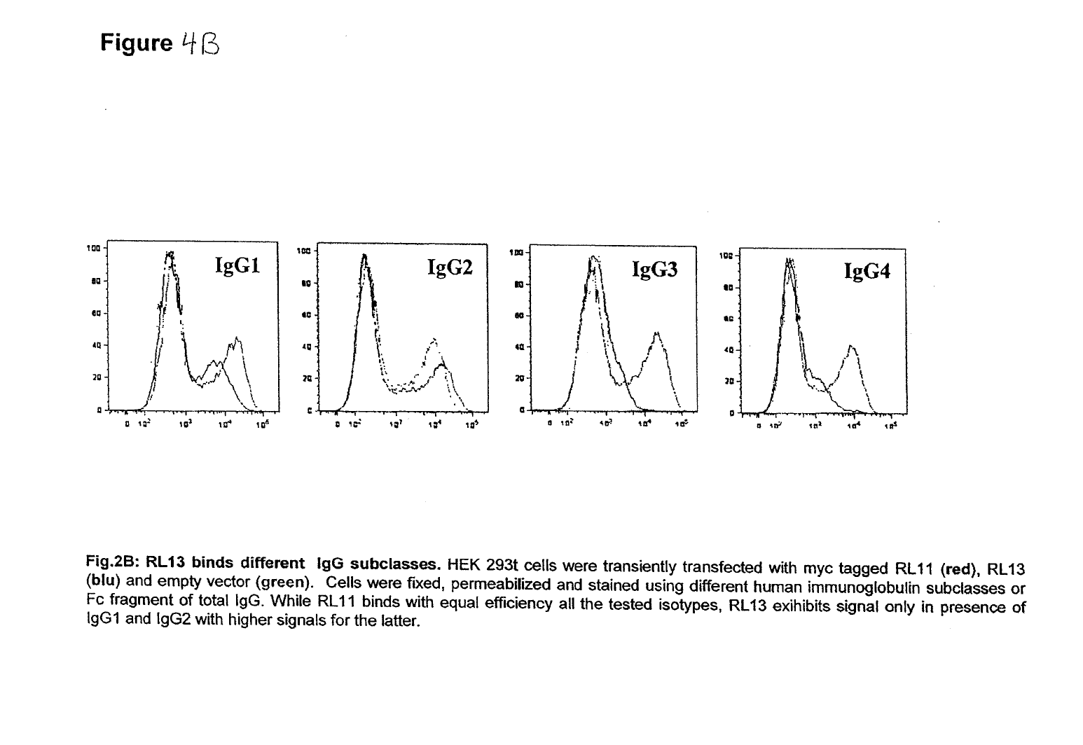

RL13, RL10, RL11 and UL119 sequences were selected from the low passage strain TR and inserted into a mammalian expression vector (pcDNA3.1) for their expression in fusion with C-terminal Myc and His tags). ARPE-19 epithelial cells were transiently transfected with these recombinant vectors, cell lysates were submitted to electrophoresis in non-reduced/non-boiled conditions and transferred to nitrocellulose membrane. FIG. 2A shows the result of the Western blot analysis using non-immune hIgG as probe and conjugated anti-human secondary antibodies to reveal. As expected, both RL11 and UL119 resulted positive at the binding to non-immune IgG (FIG. 2A, lanes 2 and 4 respectively), as well as RL10 and the lysate obtained from cells transfected with the empty vector did not show any IgG binding activity (FIG. 2A, lanes 1 and 5 respectively). The lane corresponding to the lysate from cells expressing RL13, indeed, did show an unambiguous band of approximately 100 kDa unveiling IgG binding properties (FIG. 2A, lane 3). To verify that the relative intensity of the hIgG binding signal was due to the protein properties and not to different levels of protein expression, the membrane was stripped and submitted to Western blot with anti-His antibody. The result, shown in FIG. 1B, confirms that RL13 has the ability to bind hIgG.

Results from this experiment, thus, were consistent with the identification of RL-13 as a novel hIgG binding protein coded by hCMV.

### Example 4

### Biochemical Characterization and Cellular Localization of TR Strain RL13

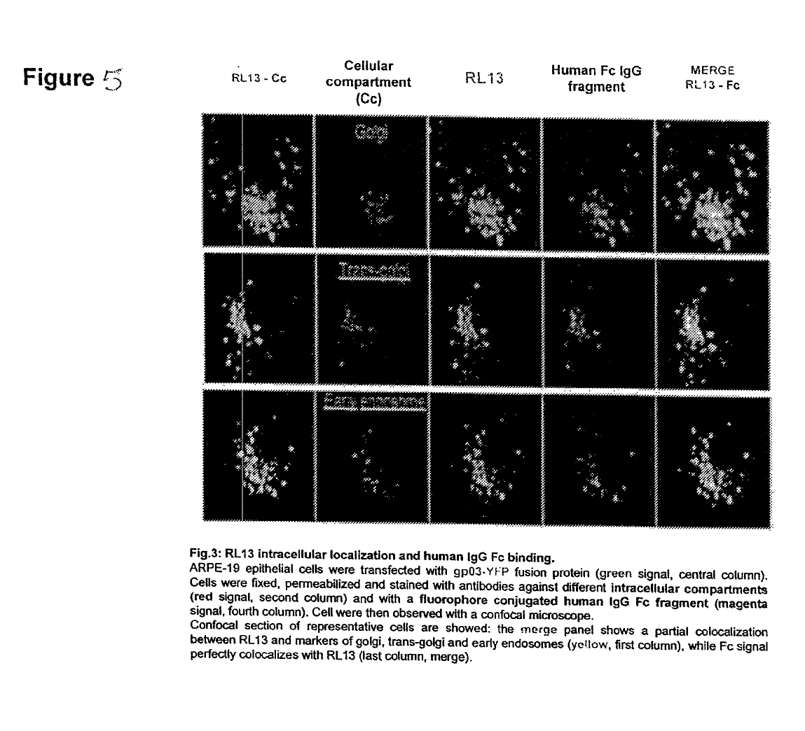

As previously shown, RL13 sequence between Merlin and TR is highly conserved, with 87% similarity. Even so, the two proteins differ in the number of potential acceptor residues of N-linked glycosylation, with 9 predicted sites for the TR against the 7 sites of the Merlin. We decided to investigate whether these differences could change the behavior of RL13 in terms of intracellular localization or glycans maturation. When TR RL13 was expressed in ARPE-19 and 293T cells using the pcDNA3.1 vector, 110-kDa, 100-kDA and 70-kDa proteins were detected (FIG. 3).

The maturation of N-linked oligosaccharides on RL13 by digestion with either Endo H, which cleaves high mannose oligosaccharides added cotranslationally in the ER, and PNGase F, which cleaves both high-mannose and Golgi-modified complex oligosaccharides, was analyzed to determine the routes of RL13 trafficking. The extracts from ARPE-19 and 293T cells were then digested with Endo H and with PNGase F. As shown in FIG. 3, the 70-kDa protein was susceptible to EndoH digestion, indicative of it being an ER-retained immature form, whereas the 110- and 100-kDa proteins were resistant to EndoH digestion and are thus presumably fully mature. Upon digestion with PNGaseF, the molecular weight of the 110 KDa and 100 KDa isoforms was reduced to 58 kDa and, in addition, a band at 38 kDa compatible with the calculated molecular weight of the RL13 protein appeared.

### Example 5

### RL13 Fc Binding Activity Analysis

ARPE-19, MRC-5 and HEK293T cells were grown respectively in DMEM:F12 (Gibco; Invitrogen) and DMEM high glucose containing 10% FCS and PSG (Gibco, Invitrogen) at 37° C. in 5% CO2.

Plasmid pcDNA3.1 mychis-C (−) containing RL10, RL11 or RL12 CMV TR genes, in frame with C-terminal myc and six histidine tags sequences (SEQ ID NO: 6), were synthesized by geneART.

Fluorescence fusion proteins of RL10, RL11 and RL12 were obtained by cloning these sequences upstream of EYFP sequence in pEYFP-N1 (Clontech) vector.

HEK293T cells were transfected using Lipofectamine 2000 (Invitrogen) with a DNA:Lipofectamine ratio of 2:5. ARPE-19 and MRC-5 were transfected using either Fugene6 (Roche) with a DNA:Fugene ratio or 1:6 of Nucleofector kit V (Amaxa) as suggested by the manufacturer.

For intracellular staining, HEK293T cells were transfected with either pcDNA3.1 mychis-C(−) or pEYP-N1 plasmids containing the RL10, RL11 and RL12 sequences. 48 hours post-transfection, cells were harvested with trypsin, fixed and permeabilized with Cytofix/Cytoperm kit (BD) as suggested by the manufacturer. For cells expressing the myc tagged proteins, anti-myc-FITC antibody (Invitrogen) was used at 1:500 dilution. To assess the binding towards human IgG Fc portion, human IgG Fc fragment 649 conjugated (Jackson immunoresearch) was used at different dilutions starting from 50 μg/ml to 1 μg/ml.

To verify the ability of RL13 to bind different human IgG isotopes, human IgG1, IgG2, IgG3 and IgG4 (SIGMA) were used at the same dilutions as above mentioned. An Alexa-Fluor goat anti-human 647 fluorophore conjugated was used as secondary antibody at 1:200 dilution.

Samples were measured on a FACSCalibur (BD), and data were analyzed in FlowJo (Treestar).

Cells were transiently transfected with the genes of interest, as above mentioned. 24 hours post transfection, they were trypsin detached and plated on glass coverslips. For intracellular staining, cells were fixed 48 hours post transfection with 3.7% paraformaldehyde. Fixed cells were then detergent permeabilized with 0.1% Triton X-100 (Sigma) and stained for 1 hour with primary antibodies. Upon washing, secondary antibodies were incubated for 1 hour, then washed again and mounted using ProLong Gold antifade reagent with DAPI (Invitrogen).

For membrane staining, 48 hours post transfection cells were treated as above mentioned without the fixation and permeabilization steps. All membrane staining were performed at 4° C.

Primary antibodies used in these experiments were mouse anti-myc-FITC, mouse anti-PDI (Invitrogen), mouse anti-GM130, mouse anti-TGN46 and mouse anti-EEA1 (Abcam), human IgG Fc fragment 649 conjugated (Jackson immunoresearch). Secondary antibodies were anti mouse IgG-Alexa Fluor 488, 568 and 647 (Invitrogen).

For human IgG internalization experiments, cells were transfected either with plasmid coding RL13 of empty vector (control) and transferred on glass coverslips 24 hours post transfection. 24 hours later, cells were washed in cold PBS and incubated at 4° C. with human IgG Fc fragment 649 fluorophore conjugated for 30 minutes.

Cells were either fixed (time 0) or incubated at 37° C. and fixed at different time points.

The intracellular locations of antibody-tagged or fluorescent fusion proteins were examined under laser illumination in a Zeiss LMS 710 confocal microscope and images were captured using ZEN software (Carl Zeiss).

HEK293T were transfected with plasmids with the genes of interests. 48 hours post transfections cells were washed in PBS and fresh culture media containing biotinylated human IgG Fc fragment (bFc) at a concentration of 10 μg/ml was supplemented. After 1 hour of 37° C. incubation, cells were harvested, washed in cold PBS several times and lysed in lysis buffer containing 1% nonidet NP-40 (Roche), 150 mM NaCl, 1 mM EDTA, 25 mM Tris-HCl pH7.4. After 30 minutes of 13000 rpm centrifugation at 4° C., supernatants were collected and incubated with 30 μl of Streptavidin Dynabeads (Invitrogen) prewashed in lysis buffer. Precipitation was carried at 4° C. for 2 hours with overnight rotation. Immunocomplexes were collected using magnetic beads, washed 4 times with lysis buffer and eluted by adding LDS-buffer and heating at 96° C. for 5 minutes.

Immunoprecipitated samples were analyzed through SDS-PAGE and western blotting.

Samples were prepared adding LDS (Invitrogen) and 100 mM DTT (Sigma) and heated at 96° C. for 3 minutes (reduced and denaturated condition).

Protein samples were then separated by SDS-PAGE using Invitrogen 4%-12% Bis-Tris NuPAGE protein gels according to the manufacturer's instructions. Gels were transferred to a nitrocellulose membrane using the P3 of the Iblot apparatus (Invitrogen) and membranes were blocked in blocking buffer (5% w/v nonfat dry milk in PBS with 0.1% Tween 20). Incubation with primary antibody in blocking buffer was done for 1 hour at room temperature or overnight at 4° C. Following 3 washes in PBST (PBS with 0.1% Tween 20), secondary antibody was incubated for 1 hour. After extensively washing in PBST, bound antibody was detected using ECL-Western blotting detection system (Amersham) or SuperSignal West Pico Chemiluminescent Substrate (Pierce) and exposure to film. Primary antibodies used were mouse anti-His(C-term) (Invitrogen), goat anti-human-HRP conjugated (Perkin Elmer). Secondary antibodies were goat anti-mouse-HRP conjugated (Perkin Elmer)

Results

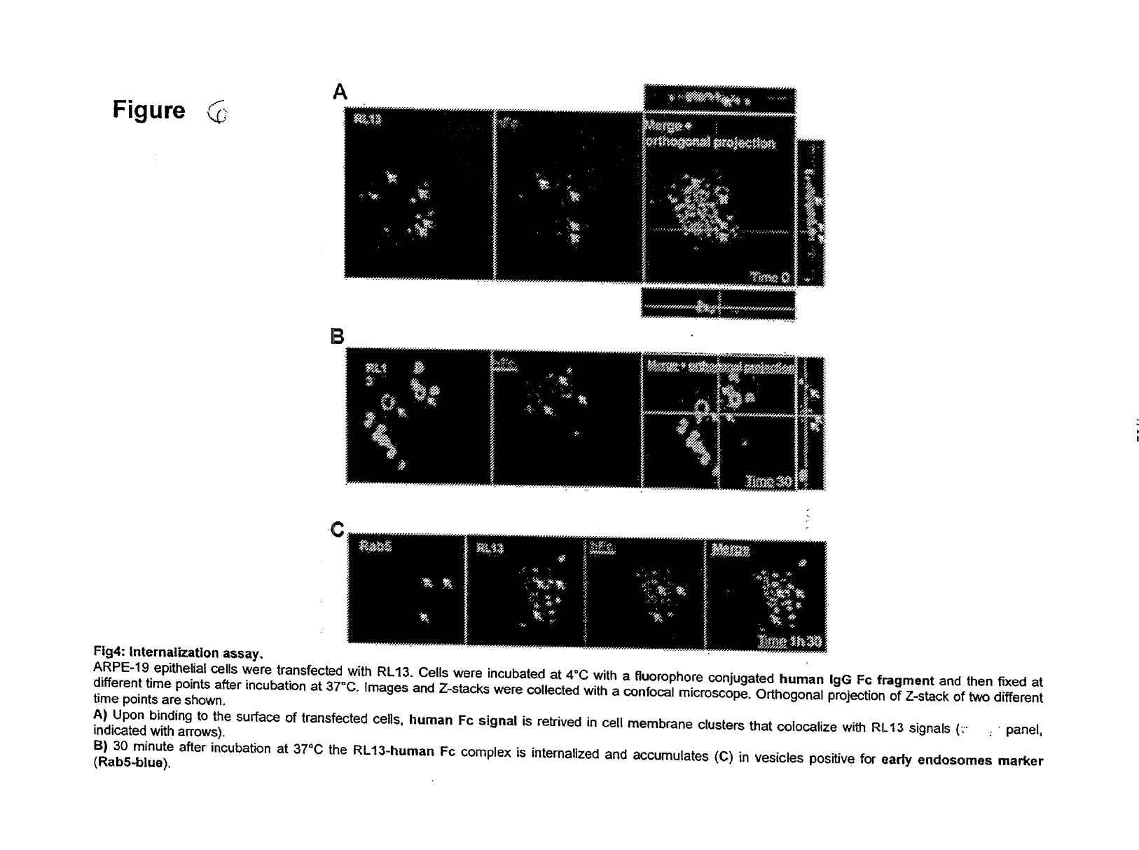

Expression of myc tagged RL10, RL11, RL12, RL13 in HEK 293T cells was obtained by transfection. Mock transfected cells were also used as control. 48 hours after transfection, cells were fixed, permeabilized and stained using both anti-myc FITC conjugated antibodies and human IgG Fc fragment (hFc) Alexa fluor 647 conjugated. FITC positive cells were compared to mock transfected cells for their ability to bind hFc (FIG. 4A).

RL11, RL12 and RL13 were able to bind the Fc portion of immunoglobulins.

RL11 has been shown to bind all different isotypes of human IgGs (Atalay, Zimmermann et al. 2002). To assess if RL13 differentially recognized human IgG isotypes, FACS analysis on RL11 and RL13 HEK 293T transfected cells was performed using individual human IgG isotypes as probe. RL11 binding to all IgG isotypes was confirmed, whereas RL13 appeared to be specific for IgG2 and, with less extent, for IgG1 (FIG. 4B).

48 hours after transfection, HEK 293T cells were fixed, permeabilized and stained with different markers of compartments and with fluorophore conjugated human IgG Fc fragment (hFc). Then, confocal microscopy analysis was performed. RL13 partially colocalized with markers of all three compartments: golgi, trans-golgi network and recycling endosomes. Co-localization with Fc was found in RL13 species present in the golgi and cytoplasmic vesicles both of the TGN and the recycling endosomes.

To investigate the RL13 membrane distribution, RL13 expressing cells were stained with fluorescent hFc. ARPE-19 cells transfected with YFP-tagged RL13 were initially placed on ice to reduce lateral diffusion of membrane proteins and also to block potential internalization of the ligand by RL13. Fluorescent labeled hFc was added and binding allowed for 30 min on ice. Following extensive washing of the hFc excess, internalization processes were restored by incubating cells at 37° C. for 30 and 90 min respectively. Finally, fixation, staining with florescent antibodies and confocal analysis was performed (FIG. 5).

In vivo labeling at low temperature showed that RL13 and hFc co-localized completely on the membrane of RL13 transfected cells, while no hFc was present on the membrane of control ARPE-19. The membrane exposed RL13 were organized in clusters. These structures could not be induced by the binding of the hFc due to the block of the lateral diffusion induced by the cold temperature. The temperature switch at 37° C. induced the internalization of the complex the majority of which accumulated mostly in large ring-shaped structures within 30 minutes, which also included the Rab5 marker, a regulator of early endosome trafficking (FIG. 6).

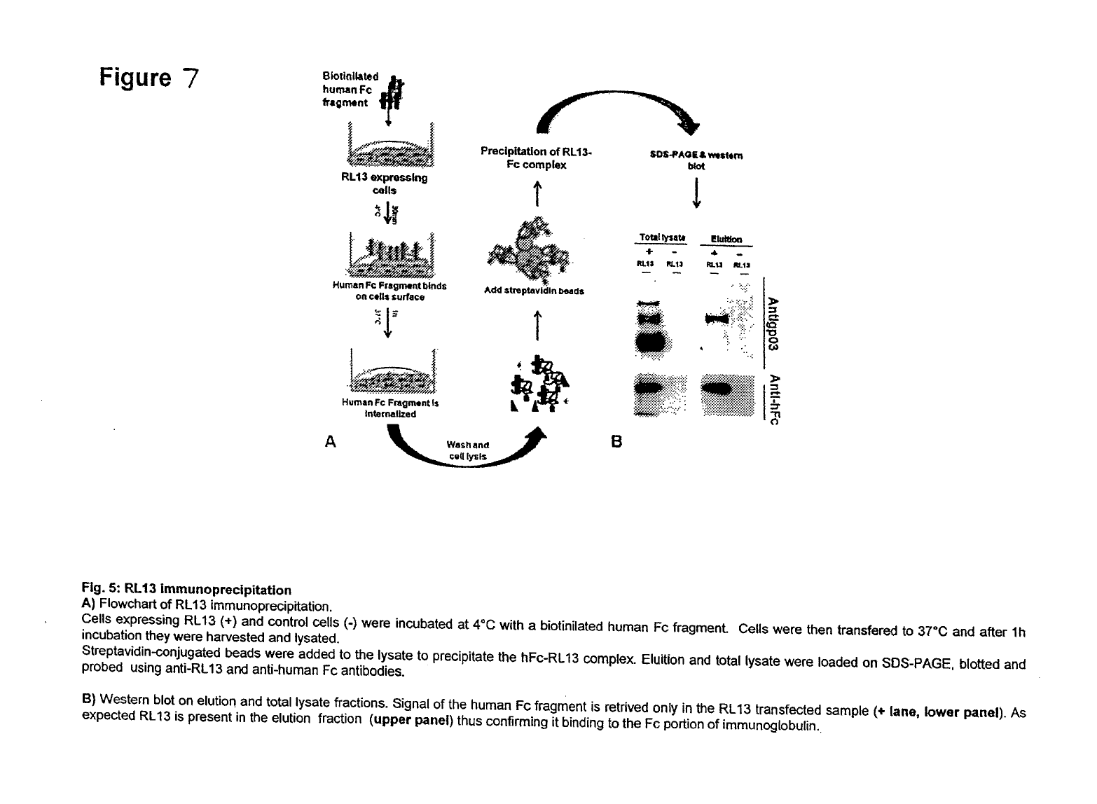

Expression of myc tagged RL13 protein in HEK 293T cells was obtained by transfection. Mock transfected cells were also used as control. 48 hours after transfection, cells were washed and culture media, supplemented with a biotinylated human IgG Fc fragment (bFc) at a concentration of 10 μg/ml, was added to each well. Cells were incubated for 1 hour at 37° C. to allow the internalization of the RL13-bFc complex and then detached in lysis buffer. Precipitation of the RL13-bFc complex was carried out using streptavidin conjugated magnetic beads at 4° C. for 2 hours. Immunocomplexes were analyzed through SDS-PAGE and western blotting. Fc binding was exclusive for RL13 expressing cells and the resulting complex can be successfully immunoprecipitated (FIG. 7).

### Example 6

### Protein-Protein Complexes Identification

ARPE-19, and HEK293T cells were grown respectively in DMEM:F12 (Gibco; Invitrogen) and DMEM high glucose containing 10% FCS and PSG (Gibco, Invitrogen) at 37° C. in 5% CO2.

Plasmid pcDNA3.1 mychis-C(−) containing RL10, RL11, RL13, UL80.5, UL122, UL138 or UL119 CMV TR genes, in frame with C-terminal myc and six histidine tags (SEQ ID NO: 6) sequences, were synthesized by geneART.

Plasmid pcDNA3.1 mychis-C(−) containing RL10, RL11, RL13 or UL119 CMV TR genes in frame with C-terminal myc tag only or six histidine tag (SEQ ID NO: 6) only were obtained through site directed mutagenesis using QuikChange® Site-Directed Mutagenesis Kit (Stratagene) according to the manufacturer's protocol.

Fluorescence fusion proteins of RL11, RL13 and UL119 were obtained cloning their coding regions upstream of EYFP or ECFP sequences in pEYFP-N1 and pECFP-N1 (Clontech) vectors respectively.

HEK293T cells were transfected using Lipofectamine 2000 (Invitrogen) with a DNA:Lipofectamine ratio of 2:5. ARPE-19 were transfected using either Fugene6 (Roche) with a DNA:Fugene ratio of 1:6 or Nucleofector kit V (Amaxa) as suggested by the manufacturer.

Foester Resonance Energy Transfer (FRET) is a technique that allows the study of protein protein interactions which are in close proximity, approximately in the range of 1-10 nm. In a classical FRET experiment the non radioactive transfer of energy from a “donor” fluorophore in its excited state to an acceptor molecules is registered. The efficiency of the energy transfer is directly linked to the distance between the acceptor and the donor. In the acceptor photobleaching technique, the value that is measured is the gain in the intensity of the donor fluorescence upon “bleaching”, that means impair the ability to absorb and thus to emit light, of the acceptor. Acceptor bleaching is achieved using high laser intensity.

For FRET experiments, ARPE-19 cells transiently co-expressing both ECFP (donor) and EYFP (acceptor) fused at the C-term of either RL11, RL13 and UL119 proteins were used. As negative control, cells co-expressing EYFP and ECFP were used. ECFP proteins were used as donor while EYFP proteins were used as acceptor. Cells were plated on glass coverslips 24 hours after co-transfection, incubated at 37° C., 5% CO₂ overnight and then fixed in 3.7% paraformaldehyde for 30 minutes on ice. Glass coverslips were then mounted on microscopy slides using Mowiol mounting medium (Mowiol 4-88, glycerol, Tris-HCl 0.2M pH 8.5,). FRET experiments were performed using a Carl Zeiss LSM710 confocal microscopy.

All parameters (laser intensity, digital gain, digital offset) were adjusted to obtain a comparable signal intensity of the EYFP and ECFP fluorescence and then not changed for the entire duration of the experiment. In a typical recording session, a region to be bleached is selected and donor intensity is collected multiple times before and after the acceptor bleaching event. All the data were analyzed using ImageJ software. The FRET efficiency has been calculated plotting the intensities of the acceptor at different time points after the bleaching, against the “donor de-quenching”, which is the gain in donor intensity calculated as: Cd=(Ci−Cb)/Ci, where Cd is the calculated donor dequenching, Ci is the intensity of donor at the “i” observation time and Cb is the intensity of the donor before the acceptor bleaching event.

HEK293T were co-transfected with two different plasmids each containing one of the gene of interest. 48 hours post transfection, cells were harvested through trypsinization washed in cold PBS two times and lysed in lysis buffer containing 1% nonidet NP-40 (Roche), 150 mM NaCl, 1 mM EDTA, 25 mM Tris-HCl pH7.4 and 5% glycerol. After 30 minutes of 13000 rpm centrifugation at 4° C., supernatants were collected and total protein content was determined using BCA protein assay kit (Pierce). 100 μg of total protein was used for co-immunoprecipitation experiments. Briefly cell lysates were incubated overnight in agitation at 4° C. with anti-his antibody conjugated magnetic beads (Genscript). Beads were then washed 5 times with lysis buffer and then heated at 96° C., 3 minutes in 2×LDS sample loading buffer (Invitrogen) to elute the protein complexes. Elution, flow through and wash fractions were analyzed through SDS-PAGE and western blotting.

Samples were prepared adding LDS (Invitrogen) and 100 mM DTT (Sigma) and heated at 96° C. for 3 minutes (reduced and denaturated condition). Protein samples were then separated by SDS-PAGE using Invitrogen 4%-12% Bis-Tris NuPAGE protein gels according to the manufacturer's instructions. Gels were transferred to a nitrocellulose membrane using the P3 of the Iblot apparatus (Invitrogen) and membranes were blocked in blocking buffer (5% w/v nonfat dry milk in PBS with 0.1% Tween 20). Incubation with primary antibody in blocking buffer was done for 1 hour at room temperature or overnight at 4° C. Following 3 washes in PBST (PBS with 0.1% Tween 20), secondary antibody was incubated for 1 hour. After extensively washing in PBST, bound antibody was detected using ECL-Western blotting detection system (Amersham) or SuperSignal West Pico Chemiluminescent Substrate (Pierce) and exposure to film. Primary antibodies used were mouse anti-myc tag (Invitrogen), rabbit anti-myc tag (Abcam). Secondary antibodies were goat anti-mouse-HRP conjugated and goat anti-rabbit-HRP conjugated (Perkin Elmer).

Results

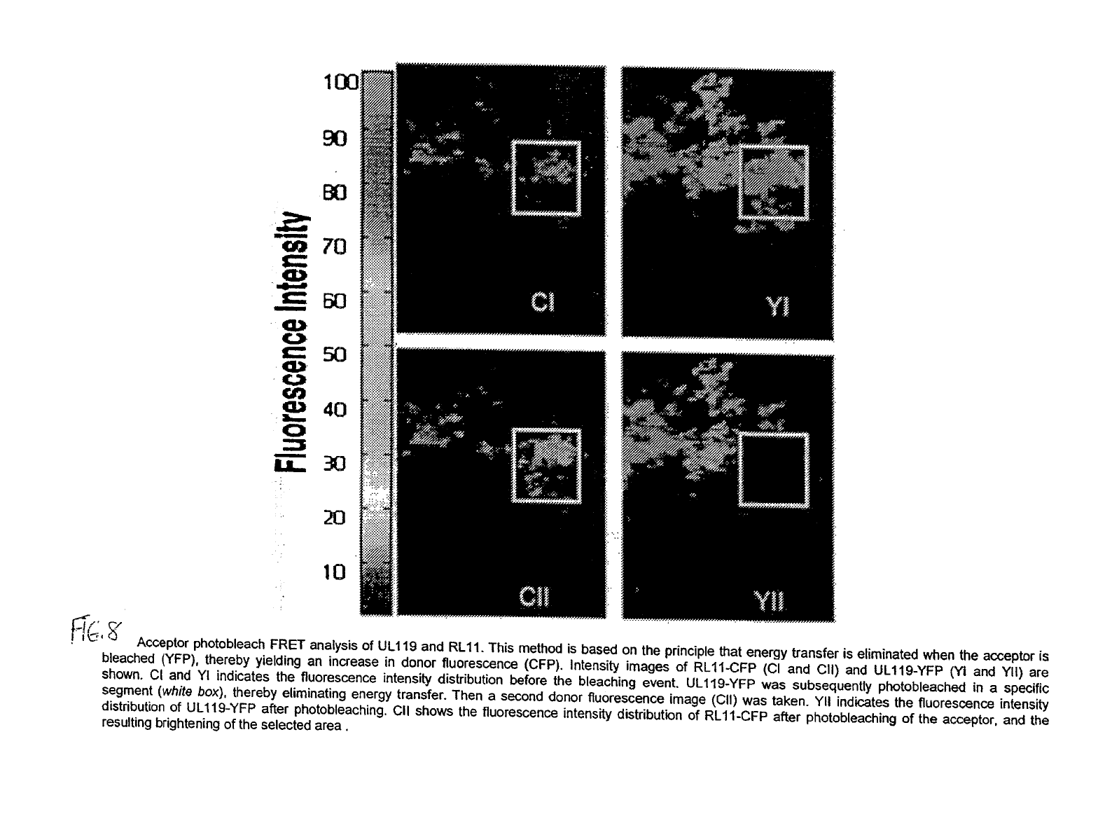

UL119 protein (also known as gp68) and RL11 protein (also known as gp34) are two known human IgG Fc binding proteins (FcBP) coded by human cytomegalovirus (Sprague et. al., 2008; Atalay et. al. 2002; Lilley et. al., 2001). So far no data are present in literature describing an interaction between UL119 and RL11. In order to verify a possible complex formation between these two proteins FRET (Foester Resonance Energy Transfer) experiments were carried out. Both UL119 and RL11 were fused at the N-terminus of both ECFP and EYFP fluorescent proteins and the resulting fusion proteins were used respectively as donor and acceptor pairs for FRET in the acceptor photobleaching approach (FIG. 8).

In this approach the intensity of the donor is calculated before and after its de-quenching upon photobleaching of the acceptor molecule. If donor and acceptor are in close proximity, an increase in donor intensity should be observed.

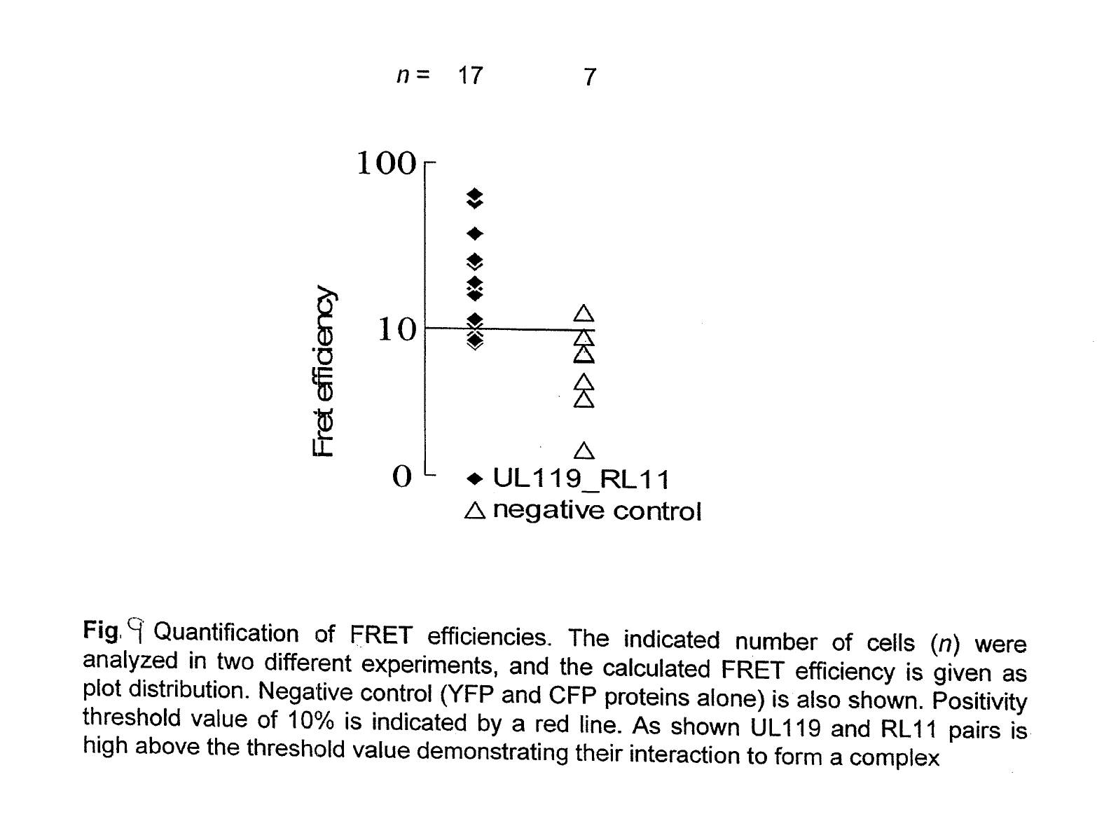

Cells used in this study were ARPE-19 epithelial cells transiently transfected with plasmids coding either for UL119-CFP and RL11-YFP or UL119-YFP and RL11-CFP. 24 hours after transfection, cells were seeded on glass coverslips overnight and then mounted on microscope slides using Mowiol mounting medium. As a control, determination of random FRET events, derived from collisions between EYFP and ECFP, was done in cells expressing the fluorescent proteins not fused to any other protein. FRET efficiency for all the samples was calculated using imageJ software. The results showed a remarkable increase in the intensity of the donor when the UL119-RL11 co-expressing samples were analyzed compared to the negative control. As already stated, the increase in the intensity is related to the close proximity of the donor/acceptor fused molecules (FIG. 9).

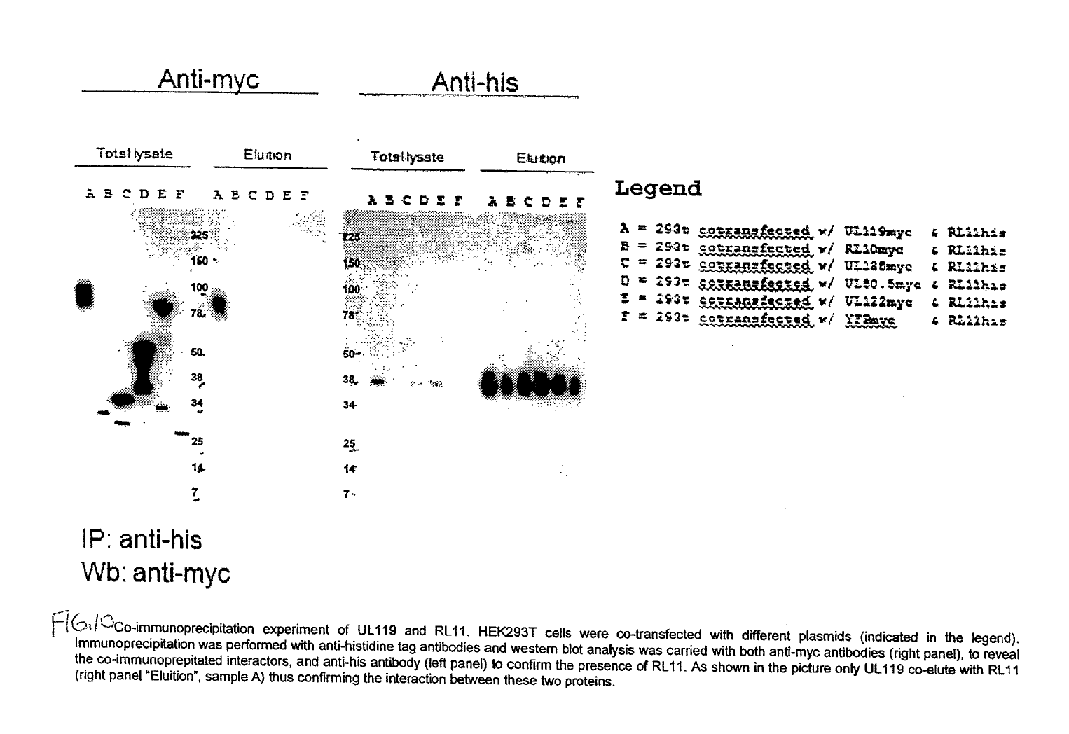

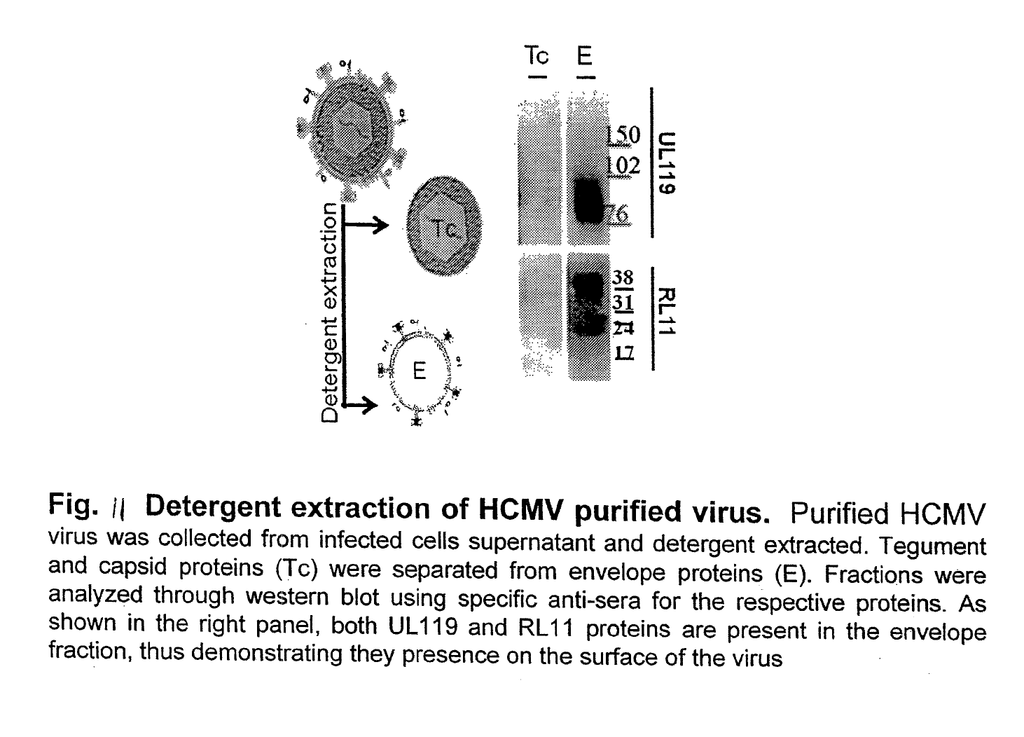

To confirm the association between UL119 and RL11 proteins co-immunoprecipitation experiments were performed. Six histidine (SEQ ID NO: 6) tagged RL11 was co-expressed either with myc tagged UL119 or with control proteins. 48 hours post transfection, cells were lysed, lysates cleared by centrifugation and total protein content dosed using BCA assay. Anti-his tag conjugated magnetic beads were incubated with 100 μg of the complexes of interest (FIG. 10).

Western blot analysis carried out using anti-myc tag antibody revealed the presence of UL119 in the elution fraction, while all controls tested resulted negative. Moreover anti-his antibody confirmed the presence of RL11 in all the tested samples thus validating the reliability of the experiment.

### Example 7

### Identification of Viral Envelope Proteins

Human cytomegalovirus TB40E-UL32GFP strain was used to infect MRC-5 cells. Supernatant from 5 to 7 days post infection was collected, clarified through centrifugation at 10000 g for 10 minutes. Cell debris-free supernatant were collected, underlined with 20% sucrose and concentrated through ultra-centrifugation at 40 minutes at 70,000×g, 16° C.

For subparticular fractioning, virus pellets were resuspended in PBS 2% NP-40 0.5% sodium deoxycholate and incubated on ice for 45 minutes. Then the samples were spun down, thereby separating a detergent phase containing the envelope proteins (oil phase) from a pellet containing the tegument and capsid proteins (water phase). Both fractions were precipitated with acetone and protein pellets were resuspended in 20 mM ammoniumbicarbonate. After addition of DTT and LDS, samples were boiled and loaded on SDS-PAGE. Western blot was performed on nitrocellulose membrane using Invitrogen Iblot system. Membrane was blocked for 1 hour in blocking buffer (5% nonfat dry milk in PBS+0.1% Tween 20) and then incubated with primary anti-sera diluted in blocking buffer for 1 hour. Membrane were washed with PBST (PBS+0.1% Tween 20) and incubated with secondary antibody goat anti-mouse HRP conjugated (Perkin Elmer) for 1 hour. After extensive washes, ECL (Amersham) or SuperSignal West Pico Chemiluminescent Substrate (Pierce) were used to detect antibodies upon film exposure.

Results

A subparticular fractioning of purified virus was performed to separate membrane associated proteins, thus bona fide envelope proteins, from the soluble ones. Viral envelope proteins fraction were separated from tegument and capsid proteins through an extraction in PBS 2% NP-40 0.5% sodiumdeoxycholate followed by incubation on ice for 45 minutes. Fractions were acetone precipitated and upon resuspension in an appropriate buffer, loaded on SDS-PAGE gel, blotted and probed using antibodies against UL119 and RL11. Both UL119 and RL11 were retrieved in the viral envelope fraction, suggesting that UL119 and RL11 are not only virus incorporated, but also envelope exposed proteins. CMV human IgG Fc binding protein (FcBP) UL119 and RL11 were detected in infected cells. UL119 has also been found on the virion (Varnuum et. al.) while RL11 presence on the virus was still uncharacterized. Our data are consistent with a virion localization of RL11.

SEQUENCES
RL10
Neut strain:
TB40/e-UL32-GFP
DNA:
(SEQ ID NO: 7)
ATGTATCCGCGTGTAATGCACGCGGTGTGCTTTTTAGCATTCGGCTTGGTAAGCTAC
GTGGCCTTCTGCGCCGAAACCACGGTCGCCACCAACTGTCTTGTGAAAACAGAAAA
TACCCACCTGACATGTAAGTGCAGTCCGAATAACACATCTAATACCGGCAATGGCA
GCAAGTGCCACGCGGTGTGCAAATGCCGGGTCACAGAACCCATTACCATGCTAGGC
GCATACTCGGCCTGGGGCGCGGGCTCGTTCGTGGCCACGCTGATAGTCCTGCTGGTG
GTCTTCTTCGTAATTTACGCGCGCGAGGAGGAGAAAAACAACACGGGCACCGAGGT
AGATCAATGTCTGGCCTATCGGAGCCTGACACGCAAAAAGTTGGAACAACACGCGG
CTAAAAAGCAGAACATCTACGAACGGATTCCATACCGACCCTCCAGACAGAAAGAT
AACTCCCCGTTGATCGAACCGACGGGCACAGACGACGAAGAGGACGAGGACGACG
ACGTC
Protein:
(SEQ ID NO: 8)
MYPRVMHAVCFLAFGLVSYVAFCAETTVATNCLVKTENTHLTCKCSPNNTSNTGNGSK
CHAVCKCRVTEPITMLGAYSAWGAGSFVATLIVLLVVFFVIYAREEEKNNTGTEVDQCL
AYRSLTRKKLEQHAAKKQNIYERIPYRPSRQKDNSPLIEPTGTDDEEDEDDDV
Immunization strain:
TR
DNA (codon-optimized*):
(SEQ ID NO: 9)
ATGTACCCCAGAGTGATGCACGCCGTGTGCTTTCTGGCCCTGGGCCTGATCAGCTAC
GTGGCCGTGTGCGCCGAGAACACCGTGACCACCAACTGCCTGGTCAAGACCGAGAA
TACCCACCTGACCTGCAAGTGCAACCCCAACAGCACCAGCACCAACGGCAGCAAGT
GCCACGCCATGTGCAAGTGCAGAGTGACCGAGCCCATCACCATGCTGGGCGCCTAT
TCTGCCTGGGGAGCCGGCAGCTTTGTGGCCACCCTGATCGTGCTGCTGGTCGTGTTC
TTCGTGATCTACGCCCGGGAGGAAGAGAAGAACAACACCGGCACCGAGGTGGACCA
GTGCCTGGCCTACAGAAGCCTGACCCGGAAGAAGCTGGAACAGCACGCCGCCAAGA
AGCAGAACATCTACGAGAGAATCCCTTACCGGCCCAGCCGGCAGAACGACAACAGC
CCCCTGATCGAGCCCACCGGCACAGACGACGAAGAGGACGAGGACGACGACGTG
Protein:
(SEQ ID NO: 10)
MYPRVMHAVCFLALGLISYVAVCAENTVTTNCLVKTENTHLTCKCNPNSTSTNGSKCH
AMCKCRVTEPITMLGAYSAWGAGSFVATLIVLLVVFFVIYAREEEKNNTGTEVDQCLA
YRSLTRKKLEQHAAKKQNIYERIPYRPSRQNDNSPLIEPTGTDDEEDEDDDV
RL11
Neut strain:
TB40/e-UL32-GFP
DNA:
(SEQ ID NO: 11)
ATGCAGACCTACAGCACCCCCCTCACGCTTGCCATAGTCACGTCGCTGTTTTTGTTCA
CAACTCAAGGAGGTTCATCGAACGCCGTCGAACCAACCAAAAAACCCCTAAAGCTC
GCCAACTACCGCGCCACCTGCGAGGACCGTACACGTACTCTGGTTACCAGGCTTAAC
ACTAGCCATCACAGCGTAGTCTGGCAACGTTATGATATCTACAGCAGATACATGCGT
CGTATGCCGCCACTTTGCATCATTACAGACGCCTATAAAGAAACCACGCATCAGGGT
GGCGCAACTTTCACGTGCACGCGCCAAAATCTCACGCTGTACAATCTTACGGTTAAA
GATACGGGAGTCTACCTCCTGCAGGATCAGTATACCGGCGATGTCGAGGCTTTCTAC
CTCATCATCCACCCACGCAGCTTCTGCCGAGCTTTGGAAACGCGTCGATGCTTTTAT
CCGGGACCAGGGAGAGTTGTGGTTACGGATTCCCAAGAGGCAGACCGAGCAATTAT
CTCGGATTTAAAACGCCAGTGGTCCGGCCTCTCACTTCATTGCGCCTGGGTTTCGGG
ACTGATGATCTTTGTTGGCGCACTGGTCATCTGCTTTCTGCGGTCGCAACGAATCGG
GGAACAGGACGCTGAACAGCTGCGGACGGACCTGGATACGGAACCTCTATTGTTGA
CGGTGGACGGGGATTTGGAG
Protein:
(SEQ ID NO: 12)
MQTYSTPLTLAIVTSLFLFTTQGGSSNAVEPTKKPLKLANYRAT
CEDRTRTLVTRLNTSHHSVVWQRYDIYSRYMRRMPPLCIITDAYKETTHQGGATFTCT
RQNLTLYNLTVKDTGVYLLQDQYTGDVEAFYLIIHPRSFCRALETRRCFYPGPGRVVV
TDSQEADRAIISDLKRQWSGLSLHCAWVSGLMIFVGALVICFLRSQRIGEQDAEQLRT
DLDTEPLLLTVDGDLE
Immunization strain:
TR
DNA (codon-optimized*):
(SEQ ID NO: 13)
ATGCAGACCTACAGCACCCCCCTGACCCTGGTCATCGTGACTAGCCTGTTTCTGTTC
ACAACCCAGGGCAACCTGAGCAACGCCGTGGAGCCCACCAAGAAGCCCCTGAAGCT
GGCCAACTACCGGGCCACCTGCGAGGACAGAACCAGAACCCTGGTCACCCGGCTGA
ACACCAGCCACCACAGCGTCGTGTGGCAGAGATACGACATCTACAGCCGGTACATG
CGGAGAATGCCCCCCCTGTGCATCATCACCGACGCCTACAAAGAGACAACCCACCA
GGGCGGAGCCACCTTCACCTGCACCCGGCAGAACCTGACCCTGTACAACCTGACCA
TCAAGGACACCGGCGTGTACCTGCTGCAGGACCAGTGTACAGGCGACGTGGAGGCC
TTCTACCTGATCATCCACCCCCGGTCCTTTTGCAGAGCCCTGGAAACCCGGCGGTGC
TTTTACCCTGGCCCTGGCAGAGTGGTGGTCACCGACAGCCAGGAAGCCGACCGGGC
CATCATCAGCGACCTGAAGCGGCAGTGGAGCGGCCTGTCTCTGCACTGTGCCTGGGT
GTCCGGCCTGATGATCTTCGTGGGCGCCCTCGTGATCTGCTTCCTGCGGAGCCAGAG
AATCGGCGAGCAGGACGCCGAGCAGCTGAGAACCGACCTGGACACCGAGCCTCTGC
TGCTGACCGTGGACGGCGACCTGGAA
Protein:
(SEQ ID NO: 14)
MQTYSTPLTLVIVTSLFLFTTQGNLSNAVEPTKKPLKLANYRATCEDRTRTLVTRLNTSH
HSVVWQRYDIYSRYMRRMPPLCIITDAYKETTHQGGATFTCTRQNLTLYNLTIKDTGVY
LLQDQCTGDVEAFYLIIHPRSFCRALETRRCFYPGPGRVVVTDSQEADRAIISDLKRQWS
GLSLHCAWVSGLMIFVGALVICFLRSQRIGEQDAEQLRTDLDTEPLLLTVDGDLE
RL12
Neut strain:
TB40/e-UL32-GFP
DNA:
(SEQ ID NO: 15)
ATGCGTACACAACATCGACGGCGAAACAAGTCATCGTACACGCAAATAACATGCAT
GTTTATCATTTTTTGGATTCTGCAGAAAAGCAAGTGTAACAACACCACTATCGCTAA
TACTTCCACGTCAATTACACTCACAAGCTTGATATCTACTGCACAACTAACATCTACT
TTACAAACCACCGGAATGTCTACCACTACATTCACATCCTCCGATGTCAACGCCAAC
ACATCCACAGGATTCACTGCAAGCTCTGCAAAAAGCACAGACGTGATCTCAACTATT
TCCACCATACCCACTCAAACATCTACAATTAACGCGACTGTAATGACAACCTCACCA
AACGGAGGCATGAATTTATCGACACAACATATAATCAGCAGTACCGCGACTTCGCA
AGCAACTACATCATTACCAATCAATACTAGTACAATGGTAACAAATACAACTCAAA
ACATCAGTACACCACTCCCAACTTGCTCATCATCTAATAGCACATTCAATGATACAT
CAAACAACCGTACTTGTCATGAAAACAGTACAATATCACAAGAATCTGAAACATTG
TTGAAGGCAATACAAGGAGACAATATCACTATAATACACAACCTAACCACCACATC
GTGCTACAAGACAGCTTGGCTTAGACATTTTAATATATCCACACACAGAAAATACAC
CCATCCCAACATAAAGAGTGGAAAATTTAGTAACCATTCATTAAAGATCCTCCATTC
GCGTGTACTGTGTGAGTGGCAGACACATTACCTAAAACATCACTACGATTTATGTTT
TACATGCGATCAGAATTTATCTTTGTCTCTGTACGGTCTTAATTTTACTCACTCTGGT
AAATATAGCTTTCGATGTTACAAAAGTGGCCATCCCTCTGAACAAAATCAAAATTTT
AATCTACAAGTACATCCTAGAAACAACACGAACGAGACACATGTGAACCCCTGGAT
ATGCGAAGAACCAAAGCACGAATGGGATACTTTGGCTGCTACATCTGATAAACCGA
CCAGTCATAAAGACGATACAACCACATCATCTACAGATCATCTATACCGCTATAATA
ATCATTCCAACACATCACACGGCAGACACACTACGTGGACTTTAGTGTTAATTTGTA
TAGCCTGCATTCTCCTATTTTTCGTCCGACGAGCTCTAAACAAAAAATACCATCCATT
AAGGGACGATATCAGTGAATCAGAATTCATAGTTCGATACAATCCTGAGCATGAGG
AT
Protein:
(SEQ ID NO: 16)
MRTQHRRRNKSSYTQITCMFIIFWILQKSKCNNTTIANTSTSITLTSLISTAQLTSTLQTTG
MSTTTFTSSDVNANTSTGFTASSAKSTDVISTISTIPTQTSTINATVMTTSPNGGMNLSTQ
HIISSTATSQATTSLPINTSTMVTNTTQNISTPLPTCSSSNSTFNDTSNNRTCHENSTISQES
ETLLKAIQGDNITIIHNLTTTSCYKTAWLRHFNISTHRKYTHPNIKSGKFSNHSLKILHSRV
LCEWQTHYLKHHYDLCFTCDQNLSLSLYGLNFTHSGKYSFRCYKSGHPSEQNQNFNLQ
VHPRNNTNETHVNPWICEEPKHEWDTLAATSDKPTSHKDDTTTSSTDHLYRYNNHSNT
SHGRHTTWTLVLICIACILLFFVRRALNKKYHPLRDDISESEFIVRYNPEHED
Immunization strain
TR
DNA (codon-optimized*):
(SEQ ID NO: 17)
ATGAGAGTGAACCGGCAGCGGCGGAACAACCTGACCTACCGGCAGACCGTGTACGT
GATCCTGACCTTCTACATCGTGCACCGGGGCATCTGCAACAGCACCGACACCAACA
ACAGCACCAGCACCTCCAACTCCACCGTGTCCGACACCAATGTGTATAGCACCCCTA
ACCCCCCTAGCGTGTCCAGCACCACCCTGGACACCAGCACCGACTCCCAGATCAGC
ATTGCCAGCAACACCATCAGCTCCACCACAAACACCCTGACCGCCTACAGCATCACC
ACCCTGAATACCTCCACCTCCAGCAGCACACTGACCGCCGTGAGCAGCACCCACAC
CCGGTCCAGCATCCTGAGCAACAACGCCAGCTATACCACCTCTCTGGACAATACCAC
CACCGATATCACCAGCAGCGAGAGCAGCATCAACGTGTCCACCGTGTACAATACCA
CCTACATCCCCGTGACCAGCCTGGCCATCAACTGCACCGCCACCATCAATGGCACCA
ACAACTCCAGCTCCAAGACCTGTCAGCAGGACATCGAGACAATCCCCGTGAAGTCC
ACCCCTCTGACCGCCGAGGAAGGCACCAACATCACCATCCACGGCAACGACACCTG
GGACTGCCCTGACGTGGTCTGGTACAGACACTACAACTGGTCCACCCACGGCCACC
ACATCTACCCCAACACCCACTACAAGACCCTGATCCACCGGCGGAAGATCCTGACC
AGCCACCCCATCTGCTACAGCGACAGAAGCAGCCCCACCGCCTACCACGACCTGTG
CCGGTCCTGCAACAAGACCGAGCTGCGGCTGTACGACCTGAACACCACCAACTCCG
GCCGGTACAGCAGACGGTGCTACAAGCAGTACCACCACCAGGGCCCCCACGAGGAC
GAGAACTTCGGCCTGACCGTGAACCCCCGGAACAACACCGACAACTACACCATCCC
CGTGTGCCCCAGATACGTGGAGACACAGAGCCAGGAAGATGAGCAGGACGACGAC
TACACCCTGAGCACCACCATCAACAACAACCTGATGCGCAAGACCGGCCACTACGA
CATCAGCCACGGCACCCACACAACCTGGGCCCTGATCCTGATCTGTATCGCCTGCAT
GCTGCTGTTCTTCGTGCGGAGAGCCCTGAACAAGAAGTACCGGCCCCTGCGGGACG
ATATCAGCGAGTCCAGCCTGGTGGTGCAGTATCACCCCGAGCACGAGGAC
Protein:
(SEQ ID NO: 18)
MRVNRQRRNNLTYRQTVYVILTFYIVHRGICNSTDTNNSTSTSNSTVSDTNVYSTPNPPS
VSSTTLDTSTDSQISIASNTISSTTNTLTAYSITTLNTSTSSSTLTAVSSTHTRSSILSN
NASYTTSLDNTTTDITSSESSINVSTVYNTTYIPVTSLAINCTATINGTNNSSSKTCQQD
IETIPVKSTPLTAEEGTNITIHGNDTWDCPDVVWYRHYNWSTHGHHIYPNTHYKTLIHRR
KILTSHPICYSDRSSPTAYHDLCRSCNKTELRLYDLNTTNSGRYSRRCYKQYHHQGPHED
ENFGLTVNPRNNTDNYTIPVCPRYVETQSQEDEQDDDYTLSTTINNNLMRKTGHYDISHG
THTTWALILICIACMLLFFVRRALNKKYRPLRDDISESSLVVQYHPEHED
RL13
Neut strain:
TB40/e-UL32-GFP
DNA:
(SEQ ID NO: 19)
ATGGACTGGCAGTTTACGGTTAAGTGGAGGTTACTGATCATCACGTTATCTGAAGGT
TGTAATGATACATGCCCTTGTTCGTGCAACTGCCTCACCTCCACCGCCTCAACCATC
AAAAATTCGTCTGATTTTGTCACTAACGCTACCAACATTTCAACTACTGCAAATAAA
ACCACGCACAAACCCTCTACCGCCTCGTCAGATACATCAACAATTACTCCAACGCTG
TTGGAATCACCGTCAAGCGTTACGCGAATATTAACAACGTTCTCTACCGTTCATAGT
ACCATTCCCTGGTTGAATACCAGCAACGTAACTTGCAACGGTAGTTTGTACACCATC
TATAAACAATCTAATTTAAATTACGAGGTAATTAACGTAACAGCGTATGTCGGTGGA
TACGTCACTCTGCAAAATTGCACTAGAACGGATACATGGTATGATGTAGAATGGATA
AAATATGGAACTCGTACACACCAACTGTGCAGAATTGGAAGTTATCATTCAACGTCT
CCACTAAACGGCATGTGTCTAGACTGTAACAGAACCTCTCTCACCATCTACAACGTA
ACCGTCGAACACGCTGGAAAATACGTTTTACATCGCTACATTGACGGTAAAAAGGA
AAACTACTATCTAACTGTATTATGGGGAACCACAACATCGTCTCCTATACCTGACAA
ATGCAAAACAAAAGAGGAGTCAGATCAGCACAGGCGCGGAGCGTGGGACGACGTA
ATAACAACTGTAAAAAACACTAACATTCCCCTGGGAATTCATGCTGTATGGGCGGGT
GTAGTCGTATCTGTGGCACTTGTAGCCTTATACATGGGTAGCCGTCGCGCTTCCAGG
AAACCGCGTTATAAAAAACTTCCCAAATATGATCCAGATGAGTTTTGGACTAAAACC
Protein:
(SEQ ID NO: 20)
MDWQFTVKWRLLIITLSEGCNDTCPCSCNCLTSTASTIKNSSDFVTNATNISTTANKTTH
KPSTASSDTSTITPTLLESPSSVTRILTTFSTVHSTIPWLNTSNVTCNGSLYTIYKQSNLNYE
VINVTAYVGGYVTLQNCTRTDTWYDVEWIKYGTRTHQLCRIGSYHSTSPLNGMCLDCN
RTSLTIYNVTVEHAGKYVLHRYIDGKKENYYLTVLWGTTTSSPIPDKCKTKEESDQHRR
GAWDDVITTVKNTNIPLGIHAVWAGVVVSVALVALYMGSRRASRKPRYKKLPKYDPD
EFWTKT
Immunization strain:
TR
DNA (codon-optimized*):
(SEQ ID NO: 21)
ATGCACTGGCACCTGGCCATCACCTGGACAGTGATCATCAGCACCTTCAGCGAGTGC
TGCAACCAGACCTGTCCCTGCAGCTGCGTGTGCGTGAACAGCACCACCGTGAACATC
TCCACCAACGAGACAACCAGCAAGGCCATCACCCCCACCGCCACCACCAATACCGC
CAAGACCACCTCCAGCCTGGTGATTACAACACCCAGCAGCGTGACAATCAGCAAGG
CCGTGAGCACAGCCGCCAGCAGCACCATCCTGAGCCAGACCAACCGGTCCCACACC
AGCAACGTGATCACAACCCCTAAGACCCGCTTCGAGTACAACATCACCGGCTACGT
GGGCCAGGAAGTGACCTTCAACTTCAGCGGCAGCTTCTGGTCCTACATCGAGTGGTT
CCGGTACAGCAGCCCCGGCTGGCTGTATAGCAGCGAACCCATCTGCACCGTGACCA
ACAGCTACCACCACACCTTCCCCAGAGGCACCCTGTGCTTCGACTGCAACATGACCA
AGTTCGTGATCTACGACCTGACCCTGAACGACAGCGGCAAATACGTGGTGAAGCGG
ACCCGGCACGACAACCAGTACGAGGAAGCCTGCTACAATCTGACAGTGATCTACGC
CAACACCACCGCCATCGTGACCAACCGGACCTGTGACCGGCGGCAGACCAAGAACA
CCGATACCACCAACCACGGCATCGGCAAGCACATCATCGAGACAATCAAGAAGGCC
AACATCCCCCTGGGCATTCATGCCGTGTGGGCCGGCATTGTGGTGTCTGTGGCCCTG
ATCGCCCTGTACATGGGCAACCGGCGGAGGCCCAGAAAGCCCCGGTACACCCGGCT
GCCCAAGTACGACCCCGACGAGTTCTGGACCAAGACC
Protein:
(SEQ ID NO: 22)
MHWHLAITWTVIISTFSECCNQTCPCSCVCVNSTTVNISTNETTSKAITPTATTNTAKTTS
SLVITTPSSVTISKAVSTAASSTILSQTNRSHTSNVITTPKTRFEYNITGYVGQEVTFNFSGS
FWSYIEWFRYSSPGWLYSSEPICTVTNSYHHTFPRGTLCFDCNMTKFVIYDLTLNDSGKY
VVKRTRHDNQYEEACYNLTVIYANTTAIVTNRTCDRRQTKNTDTTNHGIGKHIIETIKKA
NIPLGIHAVWAGIVVSVALIALYMGNRRRPRKPRYTRLPKYDPDEFWTKT
UL5
Neut strain:
TB40/e-UL32-GFP
DNA:
(SEQ ID NO: 23)
ATGTTTCTAGGCTACTCTGACTGTGTAGATCCCGGCTTTGCTGTATATCGTGTATCTA
GATCACGCTTGAAGCTCGTGTTGTCTTTTGTGTGGTTGGTCGGTTTGCGTCTCCATGA
TTGTGCCACGTTCGAATCCTGCTGTTACGACATCACCGAGGCGGAGAGTAACAAGGC
TATATCAAGGGACGAAGCAGCATTCACCTCCAGCGTGAGCACCCGCACACCGTCCC
TGGTGATCGCGCCGCCTCCTGACCGATCGATGCTGTTATCACGGGAGGAAGAACTCG
TTCCGTGGAGTCGTCTCATCATCACTAAGCAGTTCTACGGAGGCCTGATTTTCCACA
CCACCTGGGTTACCGGCTTCGTTTTGCTAGGACTCTTGACGCTTTTCGCCAGCCTGTT
TCGCGTGCCGCAATCCATCTGTCGTTTCTGCATAGACCGTCTCCGGGACATCGCCCG
TCCTTTGAAATACCGCTATCAACGTCTCGTCGCCACCGTG
Protein:
(SEQ ID NO: 24)
MFLGYSDCVDPGFAVYRVSRSRLKLVLSFVWLVGLRLHDCATFESCCYDITEAESNKAI
SRDEAAFTSSVSTRTPSLVIAPPPDRSMLLSREEELVPWSRLIITKQFYGGLIFHTTWVTGF
VLLGLLTLFASLFRVPQSICRFCIDRLRDIARPLKYRYQRLVATV
Immunization strain:
TR
DNA (codon-optimized*):
(SEQ ID NO: 25)
ATGTTTCTGGGCTACAGCGACTGCGTGGACCCCGGCTTCGCCGTGTACCGGGTGTCC
AGATCCCGGCTGAAGCTGGTGCTGTCCTTCGTGTGGCTCGTGGGCCTGAGACTGCAC
GACTGCGCCACCTTCGAGAGCTGCTGCTACGACATCACCGAGGCCGAGAGCAACAA
GGCCATCAGCCGGGACGAGGCCGTGTTCACCAGCAGCGTGTCCACCAGAACCCCCA
GCCTGGCCATTGCCCCCCCTCCCGATAGAAGTATGCTGCTGTCCCGGGAAGAGGAAC
TGGTGCCCTGGTCTAGACTGATCATCACCAAGCAGTTCTACGGCGGCCTGATCTTCC
ACACCACCTGGGTGACCGGCTTTGTGCTGCTGGGCCTGCTGACCCTGTTCGCCAGCC
TGTTCCGGGTGCCCCAGAGCATCTGCCGGTTCTGCATCGACCGGCTGCGGGATATCG
CCAGACCCCTGAAGTACAGATACCAGAGACTGGTCGCCACCGTG
Protein:
(SEQ ID NO: 26)
MFLGYSDCVDPGFAVYRVSRSRLKLVLSFVWLVGLRLHDCATFESCCYDITEAESNKAI
SRDEAVFTSSVSTRTPSLAIAPPPDRSMLLSREEELVPWSRLIITKQFYGGLIFHTTWVTGF
VLLGLLTLFASLFRVPQSICRFCIDRLRDIARPLKYRYQRLVATV
UL80.5
Neut strain:
TB40/e-UL32-GFP
DNA:
(SEQ ID NO: 27)
ATGTCGCACCCTCTGAGTGCTGCGGTTCCCGCCGCTACGGCTCCTCCAGGTGCTACC
GTGGCAGGTGCGTCGCCGGCTGTGCCGTCTCTAGCGTGGCCTCACGACGGAGTTTAT
TTACCCAAAGACGCTTTTTTCTCGCTACTTGGGGCCAGTCGCTCGGCAGCGCCCGTC
ATGTATCCCGGTGCCGTAGCGGCTCCTCCTTCTGCTTCGCCAGCACCGTTGCCTTTGC
CGTCTTATCCCGCGCCCTACGGCGCCCCCGTCGTGGGTTACGACCAGTTGGCGACAC
GTCACTTTGCGGAATACGTGGATCCCCATTATCCCGGGTGGGGTCGGCGTTACGAGC
CCGCGCCGCCTTTGCATTCGGCTTGTCCCGTGCCGCCGCCACCATCACCAGCCTATT
ACCGTCGGCGCGATTCTCCGGGCGGTATGGATGAACCACCGTCCGGATGGGAGCGT
TACGACGGTGGTCACCGTGGTCAGTCGCAGAAGCAGCACCGTCACGGGGGCAGCGG
TGGACACAACAAACGCCGTAAGGAAGCTGCGGCGGCGTCGTCGTCGTCCTCGGACG
AAGACTTGAGTTTCCCCGGCGAGGCCGAGCACGGCCGGGCGCGAAAGCGTCTAAAA
AGTCACGTCAATAGCGACGGTGGAAGTGGCGGGCACGCGGGTTCCAATCAGCAGCA
GCAACAACGTTACGATGAACTGCGGGATGCCATTCACGAGCTGAAACGCGATCTGT
TTGCCGCGCGGCAGAGTTCTACGTTACTTTCGGCGGCTCTCCCCGCTGCGGCCTCTTC
CTCCCCAACTACTACTACCGTGTGTACTCCCACCGGCGAGCTGACGAGTGGCGGAGG
AGAAACACCCACGGCACTTCTATCCGGAGGTGCCAAGGTAGCTGAGCGCGCTCAGG
CCGGCGTGGTGAACGCCAGTTGCCGCCTCGCTACCGCGTCGGGTTCTGAGGCGGCA
ACGGCCGGGCCCTCGACGGCAGGTTCTTCTTCCTGCCCGGCTAGTGTCGTGTTAGCC
GCCGCTGCTGCCCAAGCCGCCGCAGCTTCCCAGAGCCCGCCCAAAGACATGGTAGA
TCTGAATCGGCGGATTTTTGTGGCTGCGCTCAATAAGCTCGAG
Protein:
(SEQ ID NO: 28)
MSHPLSAAVPAATAPPGATVAGASPAVPSLAWPHDGVYLPKDAFFSLLGASRSAAPVM
YPGAVAAPPSASPAPLPLPSYPAPYGAPVVGYDQLATRHFAEYVDPHYPGWGRRYEPA
PPLHSACPVPPPPSPAYYRRRDSPGGMDEPPSGWERYDGGHRGQSQKQHRHGGSGGHN
KRRKEAAAASSSSSDEDLSFPGEAEHGRARKRLKSHVNSDGGSGGHAGSNQQQQQRYD
ELRDAIHELKRDLFAARQSSTLLSAALPAAASSSPTTTTVCTPTGELTSGGGETPTALLSG
GAKVAERAQAGVVNASCRLATASGSEAATAGPSTAGSSSCPASVVLAAAAAQAAAAS
QSPPKDMVDLNRRIFVAALNKLE
Immunization strain:
TB 40/e
DNA (codon-optimized*):
(SEQ ID NO: 29)
ATGAGCCATCCTCTGTCTGCCGCTGTGCCTGCTGCTACAGCCCCTCCTGGCGCTACA
GTGGCTGGCGCCTCTCCTGCTGTGCCTTCTCTGGCCTGGCCTCACGATGGCGTGTACC
TGCCCAAGGACGCCTTCTTTAGCCTGCTGGGCGCCTCTAGATCTGCCGCCCCTGTGA
TGTATCCTGGCGCCGTGGCCGCTCCTCCTTCTGCCTCTCCCGCCCCACTGCCTCTGCC
TAGCTACCCTGCCCCTTACGGCGCTCCCGTCGTGGGATACGACCAGCTGGCCACCAG
ACACTTCGCCGAGTACGTGGACCCTCACTACCCTGGCTGGGGCAGAAGATATGAGC
CTGCCCCCCCTCTGCATAGCGCCTGCCCCGTGCCTCCTCCTCCTAGCCCCGCCTACTA
CAGAAGAAGAGACAGCCCTGGCGGGATGGATGAGCCTCCTTCCGGCTGGGAGAGAT
ACGATGGCGGCCACCGGGGACAGAGCCAGAAGCAGCACAGACACGGCGGGTCCGG
GGGACACAACAAGCGGCGGAAAGAGGCCGCAGCCGCTTCCAGCTCCAGCTCCGACG
AGGACCTGAGCTTTCCTGGCGAGGCCGAGCACGGCAGAGCCCGGAAGAGACTGAAG
TCCCACGTGAACAGCGATGGCGGATCTGGCGGCCATGCCGGCTCTAATCAGCAGCA
GCAGCAGAGATACGACGAGCTGCGGGACGCCATCCACGAGCTGAAGCGGGACCTGT
TCGCCGCCAGACAGTCCAGCACCCTGCTGTCTGCAGCTCTCCCAGCCGCTGCCAGCA
GCTCTCCTACCACCACCACCGTGTGCACCCCTACCGGCGAGCTGACAAGCGGAGGG
GGCGAGACACCTACCGCTCTGCTGTCCGGCGGAGCCAAAGTGGCCGAAAGGGCCCA
GGCTGGCGTGGTCAATGCTTCCTGTAGACTGGCCACAGCCAGCGGCTCTGAAGCCGC
CACAGCCGGCCCTAGCACAGCCGGCAGCAGCTCTTGTCCTGCCTCTGTGGTGCTGGC
AGCTGCTGCAGCTCAGGCTGCTGCCGCCTCCCAGAGCCCCCCCAAGGACATGGTGG
ACCTGAACCGGCGGATCTTCGTGGCCGCCCTGAACAAGCTGGAA
Protein:
(SEQ ID NO: 30)
MSHPLSAAVPAATAPPGATVAGASPAVPSLAWPHDGVYLPKDAFFSLLGASRSAAPVM
YPGAVAAPPSASPAPLPLPSYPAPYGAPVVGYDQLATRHFAEYVDPHYPGWGRRYEPA
PPLHSACPVPPPPSPAYYRRRDSPGGMDEPPSGWERYDGGHRGQSQKQHRHGGSGGHN
KRRKEAAAASSSSSDEDLSFPGEAEHGRARKRLKSHVNSDGGSGGHAGSNQQQQQRYD
ELRDAIHELKRDLFAARQSSTLLSAALPAAASSSPTTTTVCTPTGELTSGGGETPTALLSG
GAKVAERAQAGVVNASCRLATASGSEAATAGPSTAGSSSCPASVVLAAAAAQAAAAS
QSPPKDMVDLNRRIFVAALNKLE
UL116
Neut strain:
TB40/e-UL32-GFP
DNA:
(SEQ ID NO: 31)
ATGAAGCGGCGGCGGCGATGGCGGGGCTGGTTGCTTTTCCTGGCCCTGTGCTTTTGC
TTACTGTGTGAAGCGGTGGAAACCAACGCGACCACCGTTACCAGTACCACCGCTGC
CGCCGCCACGACAAACACTACCGTCGCCACCACCGGTACCACTACTACCTCCCCTAA
CGTCACTTCAACCACGAGTAACACCGTCATCACTCCCACCACGGTTTCCTCGGTCAG
CAATCTGACATCCAGCGCCACGTCGATTCCCATCTCAACGTCAACGGTTTCTGGAAC
AAGAAACACAAGGAATAATAATACCACAACCATCGGTACGAACGTTACTTCCCCCT
CCCCTTCTGTATCCATACTTACCACCGTGACACCGGCCGCGACTTCTACCACCTCCA
ACAACGGGGATGTAACATCCGACTACACTCCAACTTTTGACCTGGAAAACATTACCA
CCACCCGCGCTCCCACGCGTCCTCCCGCCCAGGACCTTTGTAGCCATAACCTGTCAA
TCATCCTGTACGAAGAGGAATCTCAGAGCAGCGTAGACATTGCGGTGGATGAAGAA
GAGCCAGAACTGGAGGACGACGACGAGTACGACGAACTGTGGTTCCCCCTCTACTT
CGAGGCTGAGTGCAACCTAAATTACACGCTACAATACGTCAATCACAGTTGTGATTA
CAGCGTGCGCCAGTCGTCTGTCTCATTCCCCCCGTGGCGCGACATCGACTCAGTTAC
CTTCGTACCCAGGAACCTCTCCAACTGTAGCGCCCACGGTCTGGCCGTCATCGTCGC
GGGTAACCAAACCTGGTACGTGAATCCGTTTAGCCTGGCTCACCTGCTGGATGCAAT
ATATAACGTTTTAGGGATCGAAGACCTGAGCGCCAACTTTCGGCGCCAACTGGCTCC
TTACCGTCACACTCTCATCGTGCCGCAGACT
Protein:
(SEQ ID NO: 32)
MKRRRRWRGWLLFLALCFCLLCEAVETNATTVTSTTAAAATTNTTVATTGTTTTSPNVTS
TTSNTVITPTTVSSVSNLTSSATSIPISTSTVSGTRNTRNNNTTTIGTNVTSPSPSVSILTT
VTPAATSTTSNNGDVTSDYTPTFDLENITTTRAPTRPPAQDLCSHNLSIILYEEESQSSVDI
AVDEEEPELEDDDEYDELWFPLYFEAECNLNYTLQYVNHSCDYSVRQSSVSFPPWRDID
SVTFVPRNLSNCSAHGLAVIVAGNQTWYVNPFSLAHLLDAIYNVLGIEDLSANFRRQLA
PYRHTLIVPQT
Immunization strain:
TR
DNA (codon-optimized*):
(SEQ ID NO: 33)
ATGAAGCGGCGGAGAAGATGGCGGGGCTGGCTGCTGTTCCTGGCCCTGTGCTTCTGT
CTGCTGTGCGAGGCCGTGGAGACAAACGCCACCACCGTGACCGGAACAACAGCCGC
CGCTGCCACCACCAATACCACTGTCGCCACCACCGGCACCACCACCACCTCCCCCAA
CGTGACCAGCACCACAAGCAACACCGTGACCACCCCTACCACCGTGTCCAGCGTGT
CCAACCTGACCTCCAGCACAACCTCCATCCCCATCAGCACCAGCACCGTGTCCGGCA
CCCGGAACACCGGCAACAACAATACCACCACCATCGGGACTAACGCTACCTCTCCC
AGCCCTTCCGTGAGCATCCTGACCACAGCCACCCCAGCCGCTACCTCCACAACCAGC
AACAACGGCGACGTGACCTCCGACTACACCCCCACCTTCGACCTGGAAAACATCAC
CACCACAAGAGCCCCTACCAGACCCCCTGCCCAGGATCTGTGCAGCCACAACCTGA
GCATCATCCTGTACGAGGAAGAGTCCCAGAGCAGCGTGGATATCGCCGTGGACGAG
GAAGAACCCGAGCTGGAAGATGACGACGAGTACGACGAGCTGTGGTTCCCCCTGTA
CTTCGAGGCCGAGTGCAACCTGAACTACACCCTGCAGTACGTGAACCACAGCTGCG
ACTACAGCGTGCGGCAGTCCTCCGTGAGCTTCCCCCCCTGGCGGGACATCGACAGCG
TGACCTTCGTGCCCCGGAACCTGAGCAATTGCAGCGCCCACGGCCTGGCTGTGATCG
TGGCCGGCAACCAGACTTGGTACGTGAATCCCTTCAGCCTGGCCCACCTGCTGGACG
CCATCTACAACGTGCTGGGCATCGAGGACCTGAGCGCCAACTTCAGACGGCAGCTG
GCCCCCTACAGACACACCCTGATCGTGCCCCAGACC
Protein:
(SEQ ID NO: 34)
MKRRRRWRGWLLFLALCFCLLCEAVETNATTVTGTTAAAATTNTTVATTGTTTTSPNVTS
TTSNTVTTPTTVSSVSNLTSSTTSIPISTSTVSGTRNTGNNNTTTIGTNATSPSPSVSILTT
ATPAATSTTSNNGDVTSDYTPTFDLENITTTRAPTRPPAQDLCSHNLSIILYEEESQSSVDI
AVDEEEPELEDDDEYDELWFPLYFEAECNLNYTLQYVNHSCDYSVRQSSVSFPPWRDID
SVTFVPRNLSNCSAHGLAVIVAGNQTWYVNPFSLAHLLDAIYNVLGIEDLSANFRRQLA
PYRHTLIVPQT
UL119
Neut strain:
TB40/e-UL32-GFP
DNA:
(SEQ ID NO: 35)
ATGTGTTCCGTGCTGGCGATCGCGCTCGTAGTTGCGCTCTTGGGCGACATGCACCCG
GGAGTGAAAAGTAGCACCACAAGCGCCGTCACTTCCCCTAGTAATACCACCGTCAC
GTCTACTACGTCAATAAGTACCTCTAACAACGTCAGTTCTGCTGTCACCACCACGGT
ACAAACCTCTACCTCGTCCGCCTCCACCTCCGTGATAGCCACGACGCAGAAAGAGG
GGCACCTGTATACTGTGAATTGCGAAGCCAGCTACAGCTACGACCAAGTGTCTCTAA
ACGCCACCTGCAAAGTTATCCTGTTGAATAATACCAAAAATCCAGACATTTTATCAG
TTACTTGTTATGCACGGACAGACTGCAAGGGTCCCTTCACTCAGGTGGGATATCTTA
GCGCTTTTCCCTCCAACGATAAAGGAAAACTACATCTCTCCTACAACGCTACTGCTC
AAGAGCTGCTTATCTCGGGACTCAGGCCGCAGGAGACCACTGAGTACACGTGCTCTT
TCTTCAGTTGGGGCCGCCATCACAACGCCACTTGGGACCTTTTCACCTATCCCATTTA
CGCCGTGTACGGGACTCGCTTGAACGCTACCACGATGCGGGTCCGCGTGCTGCTTCA
GGAACACGAACACTGCTTGCTCAACGGTAGCAGCCTCTATCACCCCAACAGCACCG
TGCATCTGCATCAGGGCGACCAGCTCATTCCGCCGTGGAATATTAGTAACGTGACGT
ATAACGGACAACGGTTACGCGAGTTTGTCTTCTACCTCAACGGCACGTATACTGTCG
TGCGTCTCCACGTCCAGATCGCGGGCCGAAGTTTTACCACCACCTACGTGTTTATCA
AGAGCGACCCGCTGTTCGAGGACCGGCTGCTGGCCTACGGCGTGCTGGCTTTCCTGG
TGTTCATGGTAATTATTCTTTTGTACGTGACCTACATGCTGGCGCGCCGGCGGGACT
GGTCCTATAAGAGACTGGAGGAGCCCGTTGAAGAAAAGAAACACCCGGTGCCCTAC
TTCAAGCAGTGG
Protein:
(SEQ ID NO: 36)
MCSVLAIALVVALLGDMHPGVKSSTTSAVTSPSNTTVTSTTSISTSNNVSSAVTTTVQTS
TSSASTSVIATTQKEGHLYTVNCEASYSYDQVSLNATCKVILLNNTKNPDILSVTCYART
DCKGPFTQVGYLSAFPSNDKGKLHLSYNATAQELLISGLRPQETTEYTCSFFSWGRHHN
ATWDLFTYPIYAVYGTRLNATTMRVRVLLQEHEHCLLNGSSLYHPNSTVHLHQGDQLIP
PWNISNVTYNGQRLREFVFYLNGTYTVVRLHVQIAGRSFTTTYVFIKSDPLFEDRLLAYG
VLAFLVFMVIILLYVTYMLARRRDWSYKRLEEPVEEKKHPVPYFKQW
Immunization strain:
TR
DNA (codon-optimized*):
(SEQ ID NO: 37)
ATGTGCAGCGTGCTGGCCATTGCCCTGGTGGTGGCTCTCCTGGGCGACATGCACCCC
AGAGTGAAGTCCAGCACCACCTCCGCCGTGACCAGCCCCAGCAACACCACCGTGAC
CTCCACCACCTCCATCAGCACCAGCAACAACGTCACTAGCGCTGTCACAACCACCGT
GCAGACCAGCACAAGCAGCGCCAGCACCAGCGTGATCGCCACCACCCAGAAAGAG
GGCCACCTGTACACCGTGAACTGCGAGGCCAGCTACAGCTACGACCAGGTGTCCCT
GAACGCCACCTGCAAAGTGATCCTGCTGAACAACACCAAGAACCCCGACATCCTGA
GCGTGACCTGCTACGCCAGAACCGACTGCAAGGGCCCCTTCACCCAGGTCGGCTAC
CTGAGCGCCTTCCCCAGCAACGACAAGGGCAAGCTGCACCTGAGCTACAACGCCAC
CGCCCAGGAACTGCTGATCAGCGGCCTGAGGCCCCAGGAAACCACCGAGTACACCT
GCAGCTTTTTCAGCTGGGGCAGACACCACAATGCCACCTGGGACCTGTTCACCTACC
CCATCTACGCCGTGTACGGCACCAGACTGAATGCCACCACCATGAGAGTGCGGGTG
CTGCTGCAGGAACACGAGCACTGCCTGCTGAACGGCAGCAGCCTGTACCACCCCAA
CAGCACAGTGCACCTGCATCAGGGAAACCAGCTGATTCCACCCTGGAACATCAGCA
ACGTGACCTACAACGGCCAGCGGCTGCGGGAGTTCGTGTTCTACCTGAACGGCACCT
ACACCGTCGTGCGGCTGCATGTGCAGATCGCCGGCAGATCCTTCACCACCACCTATG
TGTTCATCAAGAGCGACCCCCTGTTCGAGGACAGACTGCTGGCCTACGGGGTGCTGG
CCTTCCTGGTGTTCATGGTCATCATCCTGCTGTACGTGACATACATGCTGGCCAGAC
GGCGGGACTGGTCCTACAAGCGGCTGGAAGAACCCGTGGAGGAAAAGAAGCACCC
CGTCCCTTACTTCAAGCAG
Protein:
(SEQ ID NO: 38)
MCSVLAIALVVALLGDMHPRVKSSTTSAVTSPSNTTVTSTTSISTSNNVTSAVTTTVQTS
TSSASTSVIATTQKEGHLYTVNCEASYSYDQVSLNATCKVILLNNTKNPDILSVTCYART
DCKGPFTQVGYLSAFPSNDKGKLHLSYNATAQELLISGLRPQETTEYTCSFFSWGRHHN
ATWDLFTYPIYAVYGTRLNATTMRVRVLLQEHEHCLLNGSSLYHPNSTVHLHQGNQLIP
PWNISNVTYNGQRLREFVFYLNGTYTVVRLHVQIAGRSFTTTYVFIKSDPLFEDRLLAYG
VLAFLVFMVIILLYVTYMLARRRDWSYKRLEEPVEEKKHPVPYFKQ
UL122
Neut strain:
TB40/e-UL32-GFP
DNA:
(SEQ ID NO: 39)
ATGGAGTCCTCTGCCAAGAGAAAGATGGACCCTGACAACCCTGACGAGGGCCCTTC
CTCCAAGGTGCCACGGCCCGAGACACCCGTGACCAAGGCCACGACGTTCCTGCAGA
CTATGTTAAGGAAGGAGGTTAACAGTCAGCTGAGCCTGGGAGACCCGCTGTTCCCA
GAATTGGCCGAAGAATCTCTCAAAACCTTTGAACAAGTGACCGAGGATTGCAACGA
GAACCCCGAAAAAGATGTCCTGGCAGAACTCGGTGACATCCTCGCCCAGGCTGTCA
ATCATGCCGGTATCGATTCCAGTAGCACCGGCCCCACGCTGACAACCCACTCTTGCA
GCGTTAGCAGCGCCCCTCTTAACAAGCCGACCCCCACCAGCGTCGCGGTTACTAACA
CTCCTCTCCCCGGGGCATCCGCTACTCCCGAGCTCAGCCCGCGTAAGAAACCGCGCA
AAACCACGCGTCCTTTCAAGGTGATTATTAAACCGCCCGTGCCTCCCGCGCCTATCA
TGCTGCCCCTCATCAAACAGGAAGACATCAAGCCCGAGCCCGACTTTACCATCCAGT
ACCGCAACAAGATTATCGATACCGCCGGCTGTATCGTGATCTCTGATAGCGAGGAA
GAACAGGGTGAAGAAGTCGAAACCCGCGGTGCTACCGCGTCTTCCCCTTCCACCGG
CAGCGGCACGCCGCGAGTGACCTCTCCCACGCACCCGCTCTCCCAGATGAACCACCC
TCCTCTTCCCGATCCCTTGGGCCGGCCCGATGAAGATAGTTCCTCTTCGTCTTCCTCC
TCCTGCAGTTCGGCTTCGGACTCGGAGAGTGAGTCCGAGGAGATGAAATGCAGCAG
TGGCGGAGGAGCATCCGTGACCTCGAGCCACCATGGGCGCGGCGGTTTTGGTGGCG
CGGCCTCCTCCTCTCTGCTGAGCTGCGGCCATCAGAGCAGCGGCGGGGCGAGCACC
GGACCCCGCAAGAAGAAGAGCAAACGCATCTCCGAGTTGGACAACGAGAAGGTAC
GCAATATCATGAAAGATAAGAACACCCCCTTCTGCACACCCAACGTGCAGACTCGG
CGGGGTCGCGTCAAGATTGACGAGGTGAGCCGCATGTTCCGCAACACCAATCGCTC
TCTTGAGTACAAGAACCTGCCCTTCACGATTCCCAGTATGGACCAGGTGTTAGATGA
GGCCATCAAAGCTTGCAAAACCATGCAGGTGAACAACAAGGGCATCCAGATCATCT
ACACCCGCAATCATGAGGTGAAGAGTGAGGTGGATGCGGTGCGGTGTCGCCTGGGC
ACCATGTGCAACCTGGCCCTCTCCACTCCCTTCCTCATGGAGCACACCATGCCTGTG
ACACACCCACCCGAAGTGGCGCAGCGCACGGCCGATGCTTGTAACGAAGGCGTCAA
AGCCGCGTGGAGCCTCAAAGAATTGCACACCCACCAATTATGCCCCCGTTCTTCCGA
TTACCGCAACATGATCATCCACGCTGCCACCCCCGTGGACCTGTTGGGCGCTCTCAA
CCTGTGCCTACCCCTGATGCAAAAGTTTCCCAAACAGGTCATGGTGCGCATCTTCTC
CACCAACCAGGGTGGGTTCATGCTGCCTATCTACGAGACGGCCGCGAAGGCCTACG
CCGTGGGGCAGTTTGAGCAGCCCACCGAGACCCCTCCCGAAGACCTGGACACCCTG
AGCCTGGCCATCGAGGCAGCCATCCAGGACCTGAGGAACAAGTCTCAG
Protein:
(SEQ ID NO: 40)
MESSAKRKMDPDNPDEGPSSKVPRPETPVTKATTFLQTMLRKEVNSQLSLGDPLFPELA
EESLKTFEQVTEDCNENPEKDVLAELGDILAQAVNHAGIDSSSTGPTLTTHSCSVSSAPL
NKPTPTSVAVTNTPLPGASATPELSPRKKPRKTTRPFKVIIKPPVPPAPIMLPLIKQEDIKPE
PDFTIQYRNKIIDTAGCIVISDSEEEQGEEVETRGATASSPSTGSGTPRVTSPTHPLSQMNH
PPLPDPLGRPDEDSSSSSSSSCSSASDSESESEEMKCSSGGGASVTSSHHGRGGFGGAASS
SLLSCGHQSSGGASTGPRKKKSKRISELDNEKVRNIMKDKNTPFCTPNVQTRRGRVKIDE
VSRMFRNTNRSLEYKNLPFTIPSMDQVLDEAIKACKTMQVNNKGIQIIYTRNHEVKSEV
DAVRCRLGTMCNLALSTPFLMEHTMPVTHPPEVAQRTADACNEGVKAAWSLKELHTH
QLCPRSSDYRNMIIHAATPVDLLGALNLCLPLMQKFPKQVMVRIFSTNQGGFMLPIYET
AAKAYAVGQFEQPTETPPEDLDTLSLAIEAAIQDLRNKSQ
Immunization strain:
TR
DNA (codon-optimized*):
(SEQ ID NO: 41)
ATGGAAAGCAGCGCCAAGCGGAAGATGGACCCCGACAACCCCGATGAGGGCCCCA
GCAGCAAGGTGCCCAGACCCGAGACACCTGTGACCAAGGCCACCACCTTTCTGCAG
ACCATGCTGCGGAAAGAAGTGAACAGCCAGCTGTCCCTGGGCGACCCTCTGTTTCCC
GAGCTGGCCGAGGAAAGCCTGAAAACCTTCGAGCAGGTCACCGAGGACTGCAACGA
GAACCCCGAGAAGGACGTGCTGGCTGAACTGGGCGATATTCTGGCCCAGGCCGTGA
ACCACGCCGGCATCGATAGCAGCAGCACCGGCCACACCCTGACCACCCACAGCTGC
AGCGTGTCCAGCGCCCCTCTGAACAAGCCCACCCCCACAAGCGTGGCCGTGACCAA
CACACCTCTGCCTGGCGCCTCTGCCACACCCGAGCTGTCCCCCCGGAAGAAGCCCAG
AAAGACCACCCGGCCCTTCAAAGTGATCATCAAGCCCCCCGTGCCCCCTGCTCCTAT
CATGCTGCCCCTGCTGATTAAGCAGGAAGATATCAAGCCCGAGCCCGACTTCACCAT
CCAGTACCGGAACAAGATCATCGACACCGCCGGCTGCATCGTGATCAGCGACAGCG
AGGAAGAACAGGGCGAGGAAGTGGAGACAAGAGGCGCCACCGCCAGCAGCCCTAG
CACAGGCAGCGGCACCCCTAGAGTGACCAGCCCCACCCACCCCCTGAGCCAGATGA
ACCACCCCCCCCTGCCTGATCCTCTGGGCAGACCCGACGAGGATAGCAGCTCCAGCT
CCTCTAGCTCTTGCAGCAGCGCCAGTGATAGCGAATCAGAGTCCGAAGAGATGAAG
TGCAGCTCTGGCGGCGGAGCCAGCGTGACAAGCAGCCACCACGGCAGAGGCGGATT
TGGCGGAGCCGCCTCTTCTAGCCTGCTGTCCTGTGGCCACCAGTCCTCCGGCGGAGC
CTCTACCGGCCCCAGAAAGAAGAAGTCCAAGCGGATCAGCGAGCTGGACAACGAG
AAAGTGCGGAACATCATGAAGGACAAGAACACCCCCTTTTGCACCCCCAACGTGCA
GACCAGACGGGGCAGAGTGAAGATCGACGAGGTGTCCCGGATGTTCAGAAACACCA
ACCGGTCCCTGGAATACAAGAACCTGCCCTTCATGATCCCCAGCATGCACCAGGTGC
TGGACGAGGCCATCAAGGCCTGCAAGACCATGCAGGTCAACAACAAGGGCATCCAG
ATCATCTACACCCGGAACCACGAAGTGAAGTCCGAGGTGGACGCCGTGAGATGCAG
ACTGGGCACCATGTGCAACCTGGCCCTGAGCACCCCCTTTCTGATGGAACACACCAT
GCCCGTGACCCACCCTCCAGAGGTGGCCCAGAGAACCGCCGATGCCTGCAACGAAG
GCGTGAAGGCCGCCTGGTCCCTGAAAGAGCTGCACACACACCAGCTGTGCCCCAGA
AGCAGCGACTACCGCAACATGATCATTCACGCCGCCACCCCTGTGGATCTGCTGGGC
GCCCTGAACCTGTGCCTGCCCCTGATGCAGAAATTCCCCAAGCAGGTCATGGTCCGG
ATCTTCAGCACCAACCAGGGCGGCTTCATGCTGCCTATCTACGAGACAGCCGCCAAG
GCCTACGACGTGGGCCAGTTCGAGCAGCCTACCGAGACACCCCCCGAGGACCTGGA
TACCCTGAGCCTGGCCATCGAGGCTGCTATCCAGGACCTGCGGAACAAGAGC
Protein:
(SEQ ID NO: 42)
MESSAKRKMDPDNPDEGPSSKVPRPETPVTKATTFLQTMLRKEVNSQLSLGDPLFPELA
EESLKTFEQVTEDCNENPEKDVLAELGDILAQAVNHAGIDSSSTGHTLTTHSCSVSSAPL
NKPTPTSVAVTNTPLPGASATPELSPRKKPRKTTRPFKVIIKPPVPPAPIMLPLLIKQEDIKP
EPDFTIQYRNKIIDTAGCIVISDSEEEQGEEVETRGATASSPSTGSGTPRVTSPTHPLSQMN
HPPLPDPLGRPDEDSSSSSSSSCSSASDSESESEEMKCSSGGGASVTSSHHGRGGFGGAAS
SSLLSCGHQSSGGASTGPRKKKSKRISELDNEKVRNIMKDKNTPFCTPNVQTRRGRVKID
EVSRMFRNTNRSLEYKNLPFMIPSMHQVLDEAIKACKTMQVNNKGIQIIYTRNHEVKSE
VDAVRCRLGTMCNLALSTPFLMEHTMPVTHPPEVAQRTADACNEGVKAAWSLKELHT
HQLCPRSSDYRNMIIHAATPVDLLGALNLCLPLMQKFPKQVMVRIFSTNQGGFMLPIYE
TAAKAYDVGQFEQPTETPPEDLDTLSLAIEAAIQDLRNKS
UL132
Neut strain:
TB40/e-UL32-GFP
DNA:
(SEQ ID NO: 43)
ATGCCGGCCCCGCGGGGTCCCCTTCGCGCAACATTCCTGGCCCTGGTCGCGTTCGGG
TTGCTGCTTCAGATAGACCTCAGCGACGCTACGAATGTGACCAGCAGCACAAAAGT
CCCTACTAGCACCAGCAGCAGAAATAGCGTCGACAATGCCACGAGTAGCGGACCCA
CGACCGGGATCAACATGACCACCACCCACGAGTCTTCCGTTCACAGCGTGCGCAAT
GACGAAATCATGAAAGTGCTGGCTATCCTCTTCTACATCGTGACAGGCACCTCCATT
TTCAGCTTCATAGCGGTACTGATCGCGGTAGTTTACTCCTCGTGTTGCAAGCACCCG
GGCCGCTTTCGTTTCGCCGACGAAGAAGCCGTCAACCTGTTGGACGACACGGACGA
CAGTGGCGGTGGCAGCCCGTTTGGCAGCGGTTCCCGACGAGGTTCTCAGATCCCCGC
CGGATTTTGTTCCTCGAGCCCTTATCAGCGGTTGGAAACTCGGGACTGGGACGAGGA
GGAGGAGGCGTCCGCGGCCCGCGAGCGCATGAAACATGATCCTGAGAACGTCATCT
ATTTCAGAAAGGATGGCAACTTGGACACGTCGTTCGTGAATCCCAATTATGGGAGA
GGCTCGCCTTTGACCATCGAATCTCACCTCTCGGACAATGAGGAAGACCCCATCAGG
TACTACGTCTCGGTGTACGATGAACTGACCGCCTCGGAAATGGAAGAACCTTCGAAC
AGCACCAGCTGGCAGATTCCCAAACTAATGAAAGTTGCCATGCAACCCGTCTCGCTC
AGAGATCCCGAGTACGAC
Protein:
(SEQ ID NO: 44)
MPAPRGPLRATFLALVAFGLLLQIDLSDATNVTSSTKVPTSTSSRNSVDNATSSGPTTGIN
MTTTHESSVHSVRNDEIMKVLAILFYIVTGTSIFSFIAVLIAVVYSSCCKHPGRFRFADEE
AVNLLDDTDDSGGGSPFGSGSRRGSQIPAGFCSSSPYQRLETRDWDEEEEASAARERMK
HDPENVIYFRKDGNLDTSFVNPNYGRGSPLTIESHLDNEEDPIRYYVSVYDELTASEMEE
PSNSTSWQIPKLMKVAMQPVSLRDPEYD
Immunization strain:
TR
DNA (codon-optimized*):
(SEQ ID NO: 45)
ATGCCTGCCCCTAGAGGCCTGCTGAGAGCCACCTTCCTGGTGCTCGTGGCCTTTGGC
CTGCTGCTGCACATGGACTTCAGCGACGCCACAAACATGACCAGCAGCACCAACGT
GCCCACCTCCACCTCCAGCCGGAACACCGTGGAGAGCACCACAAGCAGCGAGCCCA
CCACCGAAACCAACATGACCACCGCCAGAGAAAGCAGCGTGCACGACGCCCGGAA
CGACGAGATCATGAAGGTGCTGGCCATCCTGTTCTACATCGTGACCGGCACCAGCAT
CTTCAGCTTTATCGCCGTGCTGATCGCCGTGGTGTACTCTAGTTGCTGCAAGCACCCC
GGCAGATTCAGATTCGCCGACGAGGAAGCCGTGAATCTGCTGGACGACACCGACGA
TAGCGGCGGCAGCAGCCCTTTTGGCAGCGGCAGCAGAAGAGGCTCTCAGATCCCTG
CCGGCTTCTGTTCTAGCAGCCCCTACCAGCGGCTGGAAACCCGGGACTGGGACGAG
GAAGAGGAAGCCAGCGCCGCCAGGGAAAGAATGAAGCATGACCCTGAGAATGTGA
TCTACTTCCGGAAGGACGGCAACCTGGACACCAGCTTCGTGAACCCCAACTACGGC
AGAGGCAGCCCCCTGACCATCGAGTCCCACCTGAGCGACAACGAAGAGGACCCCAT
CCGGTACTACGTGTCCGTGTACGACGAGCTGACCGCCAGCGAGATGGAAGAACCCA
GCAACAGCACCAGCTGGCAGATCCCCAAGCTGATGAAGGTCGCCACCCAGAGCGTG
TCCCTGAGGGACCCCGAGTACGAC
Protein:
(SEQ ID NO: 46)
MPAPRGLLRATFLVLVAFGLLLHMDFSDATNMTSSTNVPTSTSSRNTVESTTSSEPTTET
NMTTARESSVHDARNDEIMKVLAILFYIVTGTSIFSFIAVLIAVVYSSCCKHPGRFRFADE
EAVNLLDDTDDSGGSSPFGSGSRRGSQIPAGFCSSSPYQRLETRDWDEEEEASAARERM
KHDPENVIYFRKDGNLDTSFVNPNYGRGSPLTIESHLSDNEEDPIRYYVSVYDELTASEM
EEPSNSTSWQIPKLMKVATQSVSLRDPEYD
UL133
Neut strain:
TB40/e-UL32-GFP
DNA:
(SEQ ID NO: 47)
ATGGGTTGCGACGTGCACGATCCTTCGTGGCAATGCCAATGGGGCGTTCCCACGATT
ATTGTGGCCTGGATAACATGCGCGGCCCTGGGAATTTGGTGTTTGGTAGGATCACCG
AATACGTTTTCGGGACCCGGCATCGCAGCCGTAGTCGGCTGTTCTGTTTTCATGATTT
TCCTCTGCGCGTATCTCATCCGTTACCGGGAATTCTTCAAGGACTCCGTAATCGACGT
CTTCACCTGCCGATGGGTGCGCTACTGCAGCTGCAGCTGTAAGTGCAGCTGCAAATG
CATTTCGGGTCCTTGTAGCCGCTGCTGTTCAGCGTGTTACAAGGAGACGATGATTTA
CGACATGGTTCAATATGGTCATCGACGGCGTCCCGGACACGGCGACGATCCCGACA
GGGTGATCTGCGAGATAGTCGAGAGTCCCCCGGTTTCGGCGCCGACAGTATTCGTCC
CCCCGCCGTCGGAGGAGTCCCACCAGCCCGTCATCCCACCGCAGCCGCCAACACCG
ACATCGGAACCCAAACCGAAGAAAGGTAGGGCGAAAGATAAACCGAAGAGCAAAC
CGAAGGACAAACCTCCGTGCGAGCCGACGGTGAGTTCACAACCACCGTCGCAGCCG
ACGGCGATGCCCGGCGGTCCGCCCGACGCGTCTCCCCCCGCCATGCCGCAGATGCC
ACCCGGCGTGGCCGAGGCGGTACAAGCTGCCGTGCAGGCGGCCATGGCCGCGGCTC
TACAACAACAGCAGCAGCATCAGACCGGAACG
Protein:
(SEQ ID NO: 48)
MGCDVHDPSWQCQWGVPTIIVAWITCAALGIWCLVGSPNTFSGPGIAAVVGCSVFMIFL
CAYLIRYREFFKDSVIDVFTCRWVRYCSCSCKCSCKCISGPCSRCCSACYKETMIYDMV
QYGHRRRPGHGDDPDRVICEIVESPPVSAPTVFVPPPSEESHQPVIPPQPPTPTSEPKPKKG
RAKDKPKSKPKDKPPCEPTVSSQPPSQPTAMPGGPPDASPPAMPQMPPGVAEAVQAAV
QAAMAAALQQQQQHQTGT
Immunization strain:
TR
DNA (codon-optimized*):
(SEQ ID NO: 49)
ATGGGCTGTGACGTGCAGGACCCCAGCTGTCAGTGTCAGTGGGGCGTGCCTGCCATC
ATCGTGATCTGGATGATCTGTGCCGCCCTGGGCATTTGGTGTCTGGCCGGCAGCAGC
GCCAATATCTTCAGCGGCCCTGGCATTGCTGCCGTGGTCGTGTGCAGCGTGTTCATG
ATCTTTCTGTGCGCCTACCTGATCCGGTACAGAGAGTTCTTCAAGGACAGCATCATC
GACATCCTGACCTGTAGATGGGTGCGCTACTGCTCCTGCTCCTGCAAGTGCAGCTGT
AAGTGTATCAGCGGACCCTGCTCCAGATGCTGTAGCGCCTGCTACAAAGAAACCAT
GATCTACGACATGGTGCAGTACGGCCACAGAAGAAGGCCTGGCCACGGCGACGACC
CCGACAGAGTGATCTGCGAGATCGTGGAGAGCCCTCCCGTGTCCGCCCCTACCGTGT
TCGTGCCTCCTCCCTCCGAGGAATCTCACCAGCCCGTGATCCCCCCTCAGCCTCCTAC
CCCTACCAGCGAGCCCAAGCCCAAGAAGGGCAGAGCCAAGGACAAGCCCAGAGGC
AGACCTAAGAACAAGCCCCCCTGCGAGCCTACAGTGTCCAGCCAGCCCCCTAGCCA
GCCAACAGCCATGCCTGGCGGCCCTCCAGATGCCCCTCCTCCCGCCATGCCTCAGAT
GCCTCCAGGCGTGGCCGAAGCTGTGCAGGCCGCCGTGCAGACAGCTGTGGCCGCTG
CTCTGCAGCAGCAACAGCAGCACCAGACCGGCACC
Protein:
(SEQ ID NO: 50)
MGCDVQDPSCQCQWGVPAIIVIWMICAALGIWCLAGSSANIFSGPGIAAVVVCSVFMIFL
CAYLIRYREFFKDSIIDILTCRWVRYCSCSCKCSCKCISGPCSRCCSACYKETMIYDMVQ
YGHRRRPGHGDDPDRVICEIVESPPVSAPTVFVPPPSEESHQPVIPPQPPTPTSEPKPKKGR
AKDKPRGRPKNKPPCEPTVSSQPPSQPTAMPGGPPDAPPPAMPQMPPGVAEAVQAAVQ
TAVAAALQQQQQHQTGT
UL138
Neut strain:
TB40/e-UL32-GFP
DNA:
(SEQ ID NO: 51)
ATGGACGATCTGCCGCTGAACGTCGGGTTACCCATCATCGGCGTGATGCTCGTGCTG
ATCGTGGCCATTCTCTGCTATCTAGCTTACCATTGGCACGACACCTTCAAACTGGTGC
GCATGTTTTTGAGCTACCGCTGGCTGATCCGCTGTTGCGAGCTGTACGGGGAATACG
AGCGCCGGTTCGCGGACCTGTCGTCGCTGGGCCTCGGCGCCGTACGGCGGGAGTCG
GACAGACGATACCGTTTCTCCGAACGGCCCGATGAGATCTTGGTCCGTTGGGAGGA
AGTGTCTTCCCAGTGCAGCTACGCGTCGTCGCGGATAACAGACCGCCGCGCGGGTTC
ATCGTCTTCGTCGTCGGTCCACGTCGCTAACCAGAGAAACAGCGTGCCTCCGCCGGA
CATGGCGGTGACGGCGCCGCTGACCGACGTCGATCTGTTGAAACCCGTGACGGGAT
CCGCGACGCAGTTCACCACCGTAGCCATGGTACATTATCATCAAGAATACACGTGA
Protein:
(SEQ ID NO: 52)
MDDLPLNVGLPIIGVMLVLIVAILCYLAYHWHDTFKLVRMFLSYRWLIRCCELYGEYER
RFADLSSLGLGAVRRESDRRYRFSERPDEILVRWEEVSSQCSYASSRITDRRAGSSSSSSV
HVANQRNSVPPPDMAVTAPLTDVDLLKPVTGSATQFTTVAMVHYHQEYT
Immunization strain:
TR
DNA (codon-optimized*):
(SEQ ID NO: 53)
ATGGACGACCTGCCCCTGAACGTGGGCCTGCCCATCATCGGCGTGATGCTGGTGCTG
ATCGTGGCCATCCTGTGCTACCTGGCCTACCACTGGCACGACACCTTCAAGCTCGTG
CGGATGTTCCTGAGCTACCGGTGGCTGATCCGGTGTTGCGAGCTGTACGGCGAGTAC
GAGCGGAGATTCGCCGATCTGAGCAGCCTGGGCCTGGGCGCCGTGAGAAGAGAGAG
CGACCGGCGGTACAGATTCAGCGAGCGGCCCGACGAAATCCTCGTGCGCTGGGAAG
AGGTGTCCAGCCAGTGCAGCTACGCCAGCAGCCGGATCACAGACAGAAGGGCCGGC
AGCAGCAGCTCTAGCAGCGTGCACGTGGCCAACCAGAGAAACAGCGTGCCCCCTCC
CGATATGGCCGTGACCGCCCCTCTGACCGACGTGGACCTGCTGAAGCCTGTGACCGG
CAGCGCCACCCAGTTTACCACCGTGGCCATGGTGCACTACCACCAGGAATACACC
Protein:
(SEQ ID NO: 54)
MDDLPLNVGLPIIGVMLVLIVAILCYLAYHWHDTFKLVRMFLSYRWLIRCCELYGEYER
RFADLSSLGLGAVRRESDRRYRFSERPDEILVRWEEVSSQCSYASSRITDRRAGSSSSSSV
HVANQRNSVPPPDMAVTAPLTDVDLLKPVTGSATQFTTVAMVHYHQEYT
UL148A
Neut strain:
TB40/e-UL32-GFP
DNA:
(SEQ ID NO: 55)
ATGAGTTCCAGCGACAATCTCGATCCTTGGATTCCCGTGTGCGTCGTGGTGGTCATG
ACCTCCGTAGTCCTGTTCGCAGGTCTGCACGTGTACTTGTGGTACGTTCGGCGGCAG
CTGGTGGCGTTCTGCCTGGAGAAGGTGTGCGTTCGCTGCTGCGGAAAAGATGAGAC
GACGCCGCTAGTGGAGGATGCCGAACCGCCGGCGGAGCTGGAGATGGTGGAAGTGT
CGGACGAGTGTTAC
Protein:
(SEQ ID NO: 56)
MSSSDNLDPWIPVCVVVVMTSVVLFAGLHVYLWYVRRQLVAFCLEKVCVRCCGKDET
TPLVEDAEPPAELEMVEVSDECY
Immunization strain:
TR
DNA (codon-optimized*):
(SEQ ID NO: 57)
ATGAGCAGCAGCGACAACCTGGACCCCTGGATTCCCGTGTGCGTGGTGGTGGTCATG
ACTAGCGTGGTGCTGTTTGCCGGCCTGCATGTGTACCTCTGGTACGTGCGGAGACAG
CTGGTCGCCTTCTGCCTGGAAAAAGTGTGCGTGCGGTGCTGCGGCAAGGACGAGAC
AACCCCCCTGGTGGAGGATGCCGAGCCTCCCGCCGAGCTGGAAATGGTGGAGGTGT
CCGACGAGTGCTAC
Protein:
(SEQ ID NO: 58)
MSSSDNLDPWIPVCVVVVMTSVVLFAGLHVYLWYVRRQLVAFCLEKVCVRCCGKDET
TPLVEDAEPPAELEMVEVSDECY
UL7
Neut strain:
TB40/e-UL32-GFP
DNA:
(SEQ ID NO: 59)
ATGGCTTCCGACGTGGGTTCTCATCCTCTGACAGTTACACGATTCCGCTGCAAAGTG
CATCATGTGTACAATAAACTGTTGATTTTAGCTTTGTTTGCCCCCGTGATTCTGGAAT
CCGTTATCTACGTGTCCGGGCCACAGGGAGGGAACGTTACCCTGATATCCAACTTCA
CTTCAAACATCAGCGTACGGTGGTTTCGCTGGGACGGCAACGATAGCCATCTCATTT
GCTTTTACAAACGTGGAGAAGGTCTTTCTACGCCCTATGTGGGTTTAAGCTTAAGTT
GTGCGGCTAACCAGATCACCATCTTCAACCTCACGTTAAACGACTCCGGTCGTTACG
GAGCAGAAGGTTTTACGAGAAGCGGCGAAAATGAAACGTTTCTGTGGTATAATTTG
ACCGTGAAACCCAAACCTTTGGAAACTACTCCAGCTAGTAACGTAACAACCATCGTC
ACGACGACATCGACGGTGACCGGCGCGAAAAGTAACGTTACGGGGAACGCCGGTTT
AGCACCACAACTACGTGTCGTCGCTGGATTCTCCAATCAGACGCCTTTGGAAAACAA
CACGCACATGGCCTTGGTAGGTGTTGTCGTGTTTCTAGCCCTAATAGTTGTTTGTATT
ATGGGGTGGTGGAAGTTGTTGTGTAGTAAACCAAAGTTA
Protein:
(SEQ ID NO: 60)
MASDVGSHPLTVTRFRCKVHHVYNKLLILALFAPVILESVIYVSGPQGGNVTLISNFTSNI
SVRWFRWDGNDSHLICFYKRGEGLSTPYVGLSLSCAANQITIFNLTLNDSGRYGAEGFT
RSGENETFLWYNLTVKPKPLETTPASNVTTIVTTTSTVTGAKSNVTGNAGLAPQLRVVA
GFSNQTPLENNTHMALVGVVVFLALIVVCIMGWWKLLCSKPKL
Immunization strain:
TR
DNA (codon-optimized*):
(SEQ ID NO: 61)
ATGGCCTCTGATGTGGGCAGCCACCCCCTGACCGTGACCCGGTTCCGGTGCAGAGTG
CACCACGTGTACAACAAGCTGCTGATCCTGGCCCTGTTCGCCCCCGTGATCCTGGAA
AGCGTGATCTACGTGTCCGGCCCTCAGGGCGGCAATGTGACCCTGATCAGCAACTTC
ACCAGCAACATCAGCGTGCGGTGGTTCAGATGGGACGGCAACGACAGCCACCTGAT
CTGCTTCTACAAGCGGGGCGAGGGCCTGAGCACACCTTACGTGGGCCTGAGCCTGA
GCTGCGCCGCCAACCAGATCACCATCTTCAACCTGACCCTGAACGACAGCGGCAGA
TACGGCGCCGAGGGCTTCACCAGAAGCGGCGAGAACGAGACATTCCTGTGGTACAA
TCTGACCGTGAAGCCCAAGCCCCTGGAAACCACCCCTGCCAGCAACGTGACCACCA
TCGTGACCACAACCAGCACCGTGACCGGCGCCAAGTCCAACGTGACCGGCAATGCC
TCTCTGGCCCCCCAGCTGAGAGCTGTGGCCGGCTTTAGCAACCAGACCCCCCTGGAA
AACAACACCCACATGGCCCTGGTCGGCGTGGTGGTGTTTCTGGCCCTGATCGTGGTC
TGCATCATGGGGTGGTGGAAGCTGCTGTGCAGCAAGCCCGAACTG
Protein:
(SEQ ID NO: 62)
MASDVGSHPLTVTRFRCRVHHVYNKLLILALFAPVILESVIYVSGPQGGNVTLISNFTSNI
SVRWFRWDGNDSHLICFYKRGEGLSTPYVGLSLSCAANQITIFNLTLNDSGRYGAEGFT
RSGENETFLWYNLTVKPKPLETTPASNVTTIVTTTSTVTGAKSNVTGNASLAPQLRAVA
GFSNQTPLENNTHMALVGVVVFLALIVVCIMGWWKLLCSKPEL
UL40
Neut strain:
TB40/e-UL32-GFP
DNA:
(SEQ ID NO: 63)
ATGAACAAATTCAGCAACACTCGTATCGGCTTCACTTGCGCGGTTGTGGCTCCGCGG
ACTTTAATTCTGACGCTTGGACTCCTGTGTATGAGGATCAGGAGTTTATTATCTTCTC
CTGCCGAGACGACGGTAACAACCGCCGGCGTGACGTCCGCTCACGGTCCGTTATGTC
CGCTCGTGTTCCAGGGTTGGGCGTACGCCGTGTACCACCAAGGCGACATGGCCCTCA
TGACACTCGACGTGTACTGCTGCCGCCAGACCTCCAACAACACCGCCGTCGCGTTCT
CGCGTCATCTTGCCGTTAACACGCTGTTGATCGAAGTGGGTAACAACACTCGCCGCC
GTGCAGACGGAGTCTCCTGCCTGGACCATTTTCGCGCGCAACACCAGGATTGCCCGG
CCCAGACGGTGCACGTGCGCGGCGTAAACGAAAGCGCTTTTGGACTCACCCATCTG
CAGTCCTGTTGCCTGAACGAGCATTCACAACTCTCGGAGCGGGTGGCCTACCATCTG
AAGCTGCGACCCGCCACGTTCGGTCTGGAGACCTGGGCCATGTACACTGTGGGCATT
CTGGCCCTGGGGTCGTTCTCCTCCTTCTATTCCCAGATCGCTAGGAGCCTGGGGGTTC
TGCCCAACGATCATCACTACGCCTTGAAAAAGGCT
Protein:
(SEQ ID NO: 64)
MNKFSNTRIGFTCAVVAPRTLILTLGLLCMRIRSLLSSPAETTVTTAGVTSAHGPLCPLVF
QGWAYAVYHQGDMALMTLDVYCCRQTSNNTAVAFSRHLAVNTLLIEVGNNTRRRAD
GVSCLDHFRAQHQDCPAQTVHVRGVNESAFGLTHLQSCCLNEHSQLSERVAYHLKLRP
ATFGLETWAMYTVGILALGSFSSFYSQIARSLGVLPNDHHYALKKA
Immunization strain:
TR
DNA (codon-optimized*):
(SEQ ID NO: 65)
ATGAACAAGTTCAGCAACACCCGGATCGGCTTCACCTGTGCCGTGATGGCCCCCAG
AACCCTGATCCTGACCCTGGGCCTGCTGTGCATGCGGATCAGATCCCTGCTGTGCTC
CCCTGCCGAGACAACCGTGACCACCGCTGGCGCCATGTCTGCCCACGGCCCCAGAT
GCCCTCTGGTGTTCCAGGGCTGGGCCTACGCCGTGTACCATCAGGGCGACATGGCTC
TGATGACCCTGGATGTGTACTGCTGTCGGCAGACCAGCAGCAACACCGTGGTGGCCT
TCAGCCACCACCCCGCCGACAACACCCTGCTGATCGAAGTGGGCAACAACACCAGA
CGGCACGTGGACGGCATCAGCTGCCAGGACCACTTCAGAGCCCAGCACCAGGATTG
CCCTGCCCAGACAGTGCACGTGCGGGGCGTGAATGAGAGCGCCTTCGGCCTGACCC
ACCTGCAGAGCTGCTGCCTGAACGAGCACAGCCAGCTGTCCGAGAGAGTGGCCTAC
CACCTGAAGCTGAGGCCCGCCACCTTTGGCCTGGAAACCTGGGCCATGTACACCGTG
GGCATCCTGGCTCTGGGCAGCTTCAGCAGCTTCTACAGCCAGATCGCCAGATCTCTC
GGCGTGCTGCCCAACGATCACCACTACGCCCTGAAGAAGGCC
Protein:
(SEQ ID NO: 66)
MNKFSNTRIGFTCAVMAPRTLILTLGLLCMRIRSLLCSPAETTVTTAGAMSAHGPRCPLV
FQGWAYAVYHQGDMALMTLDVYCCRQTSSNTVVAFSHHPADNTLLIEVGNNTRRHVD
GISCQDHFRAQHQDCPAQTVHVRGVNESAFGLTHLQSCCLNEHSQLSERVAYHLKLRP
ATFGLETWAMYTVGILALGSFSSFYSQIARSLGVLPNDHHYALKKA
UL136
Neut strain:
TB40/e-UL32-GFP
DNA:
(SEQ ID NO: 67)
ATGTCAGTCAAGGGCGTGGAGATGCCAGAAATGACGTGGGACTTGGACGTTGGAAA
TAAATGGCGGCGTCGAAAGGCCCTGAGTCGCATTCACCGGTTCTGGGAATGTCGACT
ACGGGTGTGGTGGCTGAGTGACGCCGGCGTAAGAGAAACCGACCCACCGCGTCCCC
GACGCCGCCCGACTTGGATGACCGCGGTGTTTCACGTTATCTGTGCCGTTTTGCTTAC
GCTTATGATTATGGCCATCGGCGCGCTCATCGCGTACTTAAGATATTACCACCAGGA
CAGTTGGCGAGACATGCTCCACGATCTATTTTGCGGCTGTCATTATCCTGAGAAGTG
CCGTCGGCACCACGAGCGGCAGAGAAGCAGACGGCGAGCCATGGATGTGCCCGACC
CGGAACTCGGCGACCCGGCCCGCCGGCCGTTGAACGGGGCCATGTACTACGGCAGC
GGCTGTCGCTTCGACACGGTGGAAATGGTGGACGAGACGAGACCCGCGCCGCCGGC
GCTGTCATCGCCCGAAACCGGCGACGATAGCAACGACGACGCGGTTGCCGGCGGAG
GTGCTGGCGGGGTAACATCATCCGCGACTCGTACGACGTCGTCGAACGCGCTGCTGC
CAGAATGGATGGATGCGGTACATGTGGCGGTCCAAGCCGCCGTTCAAGCGACCGTG
CAAGTAAGTGGCCCGCGGGAGAACGCCGTATCTCCCGCTACG
Protein:
(SEQ ID NO: 68)
MSVKGVEMPEMTWDLDVGNKWRRRKALSRIHRFWECRLRVWWLSDAGVRETDPPRP
RRRPTWMTAVFHVICAVLLTLMIMAIGALIAYLRYYHQDSWRDMLHDLFCGCHYPEKC
RRHHERQRSRRRAMDVPDPELGDPARRPLNGAMYYGSGCRFDTVEMVDETRPAPPALS
SPETGDDSNDDAVAGGGAGGVTSSATRTTSSNALLPEWMDAVHVAVQAAVQATVQVS
GPRENAVSPAT
Immunization strain:
TR
DNA (codon-optimized*):
(SEQ ID NO: 69)
ATGAGCGTGAAGGGCGTGGAGATGCCCGAGATGACCTGGGACCTGGACGTGGGCAA
CAAGTGGCGGCGGAGAAAGGCCCTGAGCAGAATCCACCGGTTCTGGGAGTGCCGGC
TGAGAGTGTGGTGGCTCTCCGATGCCGGCGTGAGAGAGACAGACCCCCCCAGACCC
AGACGCAGACCCACCTGGATGACCGCCGTGTTCCACGTGATCTGCGCCGTGCTGCTG
ACCCTGATGATCATGGCCATCGGCGCCCTGATCGCCTACCTGCGGTACTACCACCAG
GACAGCTGGCGGGACATGCTGCACGACCTGTTCTGCGGCTGCCACTACCCCGAGAA
GTGCAGACGGCACCACGAGCGGCAGCGGAGAAGGCGGAGAGCCATGGACGTGCCC
GACCCTGAACTGGGCGACCCTGCCAGACGACCCCTGAACGGCGCCATGTACTACGG
CAGCGGCTGCAGATTCGACACCGTGGAGATGGTGGACGAGACAAGACCTGCCCCCC
CTGCCCTGTCTAGCCCCGAGACAGGCGACGACAGCAACGATGATGCCGTGGCAGGA
GGCGGAGCTGGCGGAGTCACCAGCAGCGCCACCAGAACCACCTCCAGCAACGCCCT
GCTGCCCAAGTGGATGGATGCCGTGCATGTGGCCGTGCAGGCCGCTGTGCAGGCTA
CAGTGCAGGTGTCCGGCCCTAGAGAAAACGCCGTGAGCCCTGCCACC
Protein:
(SEQ ID NO: 70)
MSVKGVEMPEMTWDLDVGNKWRRRKALSRIHRFWECRLRVWWLSDAGVRETDPPRP
RRRPTWMTAVFHVICAVLLTLMIMAIGALIAYLRYYHQDSWRDMLHDLFCGCHYPEKC
RRHHERQRRRRRAMDVPDPELGDPARRPLNGAMYYGSGCRFDTVEMVDETRPAPPAL
SSPETGDDSNDDAVAGGGAGGVTSSATRTTSSNALLPKWMDAVHVAVQAAVQATVQ
VSGPRENAVSPAT
UL139
Neut strain:
TB40/e-UL32-GFP
DNA:
(SEQ ID NO: 71)
ATGCTGTGGATATTAATTTTATTTGCACTCGCCGCATCGGCGAGTGAAACCACTACA
GGTACCAGCTCTAATTCCAGTCAATCTACTAGTGCTACCGCCAACACGACCGTATCG
ACATGTATTAATGCCTCTAACGGCAGTAGCTGGACAGTACCACAGCTCGCGCTGCTT
GCCGCTAGCGGCTGGACATTATCTGGACTCCTTCTCTTATTTACCTGCTGCTTTTGCT
GCTTTTGGTTAGTACGTAAAATCTGCAGCTGCTGCGGCAATTCCTCCGAGTCAGAGA
GCAAAACAACCCACGCGTACACCAATGCCGCATTCACTTCTTCCGACGCGACGTTAC
CCATGGGCACTACAGGGTCGTACACTCCCCCACAGGACGGCTCATTTCCACCTCCGC
CTCGG
Protein:
(SEQ ID NO: 72)
MLWILILFALAASASETTTGTSSNSSQSTSATANTTVSTCINASNGSSWTVPQLALLAASG
WTLSGLLLLFTCCFCCFWLVRKICSCCGNSSESESKTTHAYTNAAFTSSDATLPMGTTGS
YTPPQDGSFPPPPR
Immunization strain:
TR
DNA (codon-optimized*):
(SEQ ID NO: 73)
ATGCTGTGGATTCTGGTGCTGTTCGCCCTGGCCGCCAGCGCCAGCGAGACAACCACC
GGCACCAGCAGCAACAGCAGCCAGAGCACCAGCTCCAGCAGCACCTCCAGCAATAG
CACCGCCACCCCCACAAGCGCCAGCATCCAGTGCGTGGAGAGCTTCGGCGGCAGCA
ATTGGACAGTGGCCCAGCTGGCCCTGTTTGCTGCCAGCGGCTGGACACTGAGCGGCC
TGCTGCTGCTGTTCACCTGTTGCTTTTGCTGCTTCTGGCTGGTCCGGAAGATCTGCAG
CTGCTGCGGCAACAGCTCCGAGAGCGAGAGCAAGACCACCCACGCCTACACCAACG
CCGCCTTCACCAGCTCCGATGCCACCCTGCCTATGGGCACCACCGGCAGCTACACCC
CTCCCCAGGACGGCAGCTTCCCCCCACCTCCTAGA
Protein:
(SEQ ID NO: 74)
MLWILVLFALAASASETTTGTSSNSSQSTSSSSTSSNSTATPTSASIQCVESFGGSNWTVA
QLALFAASGWTLSGLLLLFTCCFCCFWLVRKICSCCGNSSESESKTTHAYTNAAFTSSDA
TLPMGTTGSYTPPQDGSFPPPPR
US20
Neut strain:
TB40/e-UL32-GFP
DNA:
(SEQ ID NO: 75)
ATGCAGGCGCAGGAGGCTAACGCGCTGCTGCTCTCCCGCATGGAGGCTCTCGAGTG
GTTCAAAAAGTTCACCGTATGGCTGCGCGTGTACGCCATCTTCATCTTTCAGCTGGCT
TTCAGCTTCGGCTTGGGAAGCGTTTTTTGGTTGGGGTTCCCACAAAACCGCAACTTTT
GCGTCGAGAACTACAGCTTCTTTCTCACCGTGCTCGTGCCCATCGTCTGCATGTTCAT
CACGTACACGTTGGGCAACGAACACCCTAGTAACGCCACGGTGCTTTTCATCTATCT
GTTGGCCAACAGCCTGACGGCGGCCATCTTCCAAATGTGCTCTGAAAGCCGCGTACT
AGTAGGTTCCTACGTGATGACCCTGGCGTTGTTTATCTCCTTTACGGGGCTGGCGTTT
CTAGGTGGCCGTGACCGACGTCGCTGGAAATGCATCAGCTGCGTCTACGTGGTGATG
CTGCTTTCGTTCCTCACGCTCGCTCTGCTAAGCGACGCCGATTGGCTGCAGAAGATA
GTGGTGACGTTGTGCGCCTTCTCTATCAGCTTCTTTTTGGGTATTCTGGCCTACGACA
GTCTCATGGTCATCTTTTTCTGCCCACCTAACCAATGCATCCGTCACGCCGTCTGTCT
CTACCTGGACAGCATGGCCATCTTTCTCACGTTGTTGCTCATGCTCTCGGGTCCCCGT
TGGATTAGTCTTTCGGACGGCGCGCCTTTGGACAACGGGACTTTGACAGCCGCCAGT
ACGACGGGGAAGTCC
Protein:
(SEQ ID NO: 76)
MQAQEANALLLSRMEALEWFKKFTVWLRVYAIFIFQLAFSFGLGSVFWLGFPQNRNFC
VENYSFFLTVLVPIVCMFITYTLGNEHPSNATVLFIYLLANSLTAAIFQMCSESRVLVGSY
VMTLALFISFTGLAFLGGRDRRRWKCISCVYVVMLLSFLTLALLSDADWLQKIVVTLCA
FSISFFLGILAYDSLMVIFFCPPNQCIRHAVCLYLDSMAIFLTLLLMLSGPRWISLSDGAPL
DNGTLTAASTTGKS
Immunization strain:
TB 40/e
DNA (codon-optimized*):
(SEQ ID NO: 77)
ATGCAGGCCCAGGAAGCCAACGCCCTGCTGCTGTCCCGGATGGAAGCCCTGGAATG
GTTCAAGAAGTTCACCGTCTGGCTGCGGGTGTACGCCATCTTCATCTTCCAGCTGGC
CTTCAGCTTTGGCCTGGGCAGCGTGTTCTGGCTGGGCTTCCCTCAGAACCGGAACTT
CTGCGTGGAGAACTACAGCTTCTTCCTGACCGTGCTGGTGCCCATCGTGTGCATGTT
CATCACCTACACCCTGGGCAACGAGCACCCCAGCAACGCCACCGTGCTGTTCATCTA
CCTGCTGGCCAACAGCCTGACCGCCGCCATCTTCCAGATGTGCAGCGAGAGCAGAG
TGCTCGTGGGCAGCTACGTGATGACCCTGGCACTGTTCATCAGCTTCACCGGCCTGG
CCTTTCTGGGCGGCAGAGACAGACGGCGGTGGAAGTGCATCAGCTGCGTGTACGTG
GTCATGCTGCTGTCTTTTCTGACACTGGCCCTGCTGTCCGACGCCGACTGGCTGCAG
AAAATCGTGGTCACCCTGTGCGCCTTCAGCATCAGCTTTTTTCTGGGCATCCTGGCCT
ACGACAGCCTGATGGTCATCTTCTTTTGCCCCCCCAACCAGTGCATCAGACACGCCG
TGTGCCTGTACCTGGACAGCATGGCCATCTTTCTGACTCTGCTGCTGATGCTGTCCGG
CCCCAGATGGATCAGCCTGAGCGACGGCGCTCCCCTGGATAATGGCACCCTGACAG
CCGCCAGCACCACAGGCAAGAGC
Protein
(SEQ ID NO: 78)
MQAQEANALLLSRMEALEWFKKFTVWLRVYAIFIFQLAFSFGLGSVFWLGFPQNRNFC
VENYSFFLTVLVPIVCMFITYTLGNEHPSNATVLFIYLLANSLTAAIFQMCSESRVLVGSY
VMTLALFISFTGLAFLGGRDRRRWKCISCVYVVMLLSFLTLALLSDADWLQKIVVTLCA
FSISFFLGILAYDSLMVIFFCPPNQCIRHAVCLYLDSMAIFLTLLLMLSGPRWISLSDGAPL
DNGTLTAASTTGKS
US27
Neut strain:
TB40/e-UL32-GFP
DNA:
(SEQ ID NO: 79)
ATGACCACCTCTACAAACCAAACCTTAACACAGGTGAGCAACATGACAAATCACAC
CTTGAACAACACCGAAATCTATCAGCTGTTCGAGTACACTCGGTTGGGGGTATGGTT
GATGTGCATCGTGGGCACGTTTCTGAACGTGCTGGTGATCACCACCATCATGTACTA
CCGTCGTAAGAAGAAATCTCCGAGCGATACTTACATCTGCAACCTGGCTATAGCCGA
TCTGCTGATTGTCGTCGGCCTGCCGTTTTTTCTAGAATATGCCAAGCATCACCCTAAA
CTCAGCCGAGAGGTGGTTTGTTCGGGACTCAACGCTTGTTTCTACATCTGTCTTTTTG
CCGGCGTTTGTTTTCTCATCAACCTGTCGATGGATCGCTACTGCGTCATTGTTTGGGG
TGTAGAATTGAACCGCGTGCGAAATAACAAGCGGGCCACCTGTTGGGTGGTGATTTT
TTGGATACTAGCCGTGCTTATGGGGATGCCACATTACCTGATGTACAGCCATACCAA
CAACGAGTGTGTTGGTGAATTCGCTAACGAGACTTCGGGTTGGTTCCCCGTGTTTTT
GAACACCAAAGTTAACATTTGCGGCTACCTGGCGCCCATTGCGCTGATGGCGTACAC
GTACAACCGTATGGTGCGGTTTATCATTAACTACGTTGGTAAATGGCACATGCAGAC
GCTCCACGTTCTTTTGGTTGTGGTTGTGTCTTTTGCCAGCTTTTGGTTTCCTTTCAACC
TGGCGCTATTTTTAGAATCCATCCGTCTTCTGGCGGGAGTGTACAATGACACACTTC
AAAACGTTATTATCTTCTGTCTATACGTCGGTCAGTTTTTGGCCTACGTTCGCGCTTG
TCTGAATCCTGGGATCTACATCCTAGTAGGCACTCAAATGAGGAAGGACATGTGGA
CAACCCTAAGGGTATTCGCCTGTTGCTGCGTGAAGCAGGAGATACCTTACCAGGACA
TTGATATTGAGCTACAAAAGGACATACAAAGAAGGGCCAAACACACCAAACGTACC
CATTATGACAGAAAAAATGCACCTATGGAGTCCGGGGAGGAGGAATTTCTATTG
Protein:
(SEQ ID NO: 80)
MTTSTNQTLTQVSNMTNHTLNNTEIYQLFEYTRLGVWLMCIVGTFLNVLVITTIMYYRR
KKKSPSDTYICNLAIADLLIVVGLPFFLEYAKHHPKLSREVVCSGLNACFYICLFAGVCFL
INLSMDRYCVIVWGVELNRVRNNKRATCWVVIFWILAVLMGMPHYLMYSHTNNECVG
EFANETSGWFPVFLNTKVNICGYLAPIALMAYTYNRMVRFIINYVGKWHMQTLHVLLV
VVVSFASFWFPFNLALFLESIRLLAGVYNDTLQNVIIFCLYVGQFLAYVRACLNPGIYILV
GTQMRKDMWTTLRVFACCCVKQEIPYQDIDIELQKDIQRRAKHTKRTHYDRKNAPMES
GEEEFLL
Immunization strain:
TR
DNA (codon-optimized*):
(SEQ ID NO: 81)
ATGACCACCTCCACCAACAACCAGACCCTGACCCAGGTGTCCAACATGACCAACCA
CACCCTGAACAGCACCGAGATCTACCAGCTGTTCGAGTACACCCGGCTGGGCGTGT
GGCTGATGTGCATCGTGGGCACCTTTCTGAACGTGCTGGTCATCACCACCATCCTGT
ACTACCGGCGGAAGAAGAAGTCCCCCAGCGACACCTACATCTGCAACCTGGCCGTG
GCCGACCTGCTGATCGTCGTGGGCCTGCCCTTCTTCCTGGAATACGCCAAGCACCAC
CCCAAGCTGTCCCGGGAGGTCGTGTGTAGCGGCCTGAACGCCTGCTTCTACATCTGC
CTGTTCGCCGGCGTGTGCTTCCTGATCAACCTGAGCATGGACCGGTACTGCGTGATC
GTGTGGGGCGTGGAGCTGAACAGAGTGCGGAACAACAAGCGGGCCACCTGCTGGGT
GGTCATCTTCTGGATTCTGGCCGTGCTGATGGGCATGCCTCACTACCTGATGTACAG
CCACACCAACAACGAGTGCGTGGGCGAGTTCGCCAACGAGACAAGCGGCTGGTTCC
CCGTGTTCCTGAACACCAAAGTGAACATCTGCGGCTACCTGGCCCCTATCGCCCTGA
TGGCCTACACCTACAACCGGATGGTCCGGTTCATCATCAACTACGTGGGCAAGTGGC
ACATGCAGACCCTGCACGTGCTGCTGGTCGTGGTGGTGTCCTTCGCCAGCTTCTGGT
TCCCCTTCAACCTGGCCCTGTTCCTGGAAAGCATCCGGCTGCTGGCTGGCGTGTACA
ACGACACCCTGCAGAACGTGATCATCTTCTGCCTGTACGTGGGCCAGTTCCTGGCCT
ATGTGCGGGCCTGCCTGAACCCAGGCATCTACATCCTCGTGGGCACACAGATGCGG
AAGGATATGTGGACCACCCTGCGGGTGTTCGCCTGCTGCTGCGTGAAGCAGGAAAT
CCCCTACCAGGACATCGACATCGAGCTGCAGAAGGACATCCAGCGGAGAGCCAAGA
ACACCAAGCGGACCCACTACGACAGAAAGCACGCCCCCATGGAAAGCGGCGAGGA
AGAGTTCCTGCTG
Protein:
(SEQ ID NO: 82)
MTTSTNNQTLTQVSNMTNHTLNSTEIYQLFEYTRLGVWLMCIVGTFLNVLVITTILYYRR
KKKSPSDTYICNLAVADLLIVVGLPFFLEYAKHHPKLSREVVCSGLNACFYICLFAGVCF
LINLSMDRYCVIVWGVELNRVRNNKRATCWVVIFWILAVLMGMPHYLMYSHTNNECV
GEFANETSGWFPVFLNTKVNICGYLAPIALMAYTYNRMVRFIINYVGKWHMQTLHVLL
VVVVSFASFWFPFNLALFLESIRLLAGVYNDTLQNVIIFCLYVGQFLAYVRACLNPGIYIL
VGTQMRKDMWTTLRVFACCCVKQEIPYQDIDIELQKDIQRRAKNTKRTHYDRKHAPME
SGEEEFLL
US29
Neut strain:
TB40/e-UL32-GFP
DNA:
(SEQ ID NO: 83)
ATGCGGTGTTTCCGATGGTGGCTCTACAGTGGGTGGTGGTGGCTCACGTTTGGATGT
GCTCGGACCGTGACGGTGGGTTTCGTCGCGCCCACGGTCCGGGCACAATCAACCGT
GGTCCGCTCTGAGCCGGCTCCGCCGTCGGAAACCCGACGAGACAACAATGACACGT
CTTACTTCAGCAGCACCTCTTTCCATTCTTCCGTGTCCCCTGCCACCTCAGTGGACCG
TCAATTTCGACGGACCACGTACGACCGTTGGGACGGTCGACGTTGGCTGCGCACCCG
CTACGGGAACGCCAGCGCCTGCGTGACGGGCACCCAATGGAGCACCAACTTTTTTTT
CTCTCAGTGTGAGCACTACCCTAGTTTCGTGAAACTCAACGGGGTGCAGCGCTGGAC
ACCTGTTCGGAGACCTATGGGCGAGGTTGCCTACTACGGGGGTTGTTGTATGGTGGG
CGGGGGTAATCGTGCGTACGTGATACTCGTGAGCGGTTACGGGACCGCCAGCTACG
GCAACGCTTTACGCGTGGATTTTGGGCGCGGCAACTGCACGGCGCCGAAACGCACC
TACCCTCGGCGCTTGGAACTGCACGATGGCCGCACAGACCCTAGCCGTTGCGATCCC
TACCAAGTATATTTCTACGGTCTGCAGTGTCCTGAGCAACTGGTTATCACCGCCCAC
GGCGGCGTGGGTATGCGCCGCTGTCCTACCGGCTCTCGTCCCACCCCGTCCCGGCCC
CACCGGCATGACTTGGAGAACGAGCTACATGGTCTGTGTGTGGATCTTCTGGTGTGC
GTCCTTTTATTAGCTCTGCTGCTGTTGGAGCTCGTTCCCATGGAAGCCGTGCGTCACC
CGCTGCTTTTCTGGCGACGCGTGGCGTTATCGCCGTCCACTTCCAAGGTGGATCGCG
CCGTCAAGCTGTGTCTTCGGCGCATGCTGGGTCTGCCGCCGCCACCGTCAGTCGCAC
CACCTGGGGAAAAGAAGGAGCTACCGGCTCAGGCGGCCTTGTCGCCGCCACTGACC
ACCTGGTCACTACCGCCGTTTCTGTCCACGCGGATACCTGACAGTCCGCCGCCACCG
TACCAGCTTCGTCACGCCACGTCACTAGTGACGGTACCCACGCTGCTGTTATATACG
TCATCCGACATCGGTGACACAGCTTCAGAAACAACGTGTGTGGCGCACGCTACTTAT
GGGGAACCCCCGGAGCCCGCTCGATCGACGGCTACGGTTCAGGAATGTACGGTTCT
TACCGCCCCGAATTGCGGCATCGTCAACAACGACGGCGCGGTCTCTGAAGGCCAAG
ACCATGGAGATGCGGTTCACCATAGCCTGGATGTGGTTTCCCAGTGTGCTGCTGATA
CTGGGGTTGTTGACACCTCCGAG
Protein:
(SEQ ID NO: 84)
MRCFRWWLYSGWWWLTFGCARTVTVGFVAPTVRAQSTVVRSEPAPPSETRRDNNDTS
YFSSTSFHSSVSPATSVDRQFRRTTYDRWDGRRWLRTRYGNASACVTGTQWSTNFFFSQ
CEHYPSFVKLNGVQRWTPVRRPMGEVAYYGGCCMVGGGNRAYVILVSGYGTASYGN
ALRVDFGRGNCTAPKRTYPRRLELHDGRTDPSRCDPYQVYFYGLQCPEQLVITAHGGV
GMRRCPTGSRPTPSRPHRHDLENELHGLCVDLLVCVLLLALLLLELVPMEAVRHPLLFW
RRVALSPSTSKVDRAVKLCLRRMLGLPPPPSVAPPGEKKELPAQAALSPPLTTWSLPPFL
STRIPDSPPPPYQLRHATSLVTVPTLLLYTSSDIGDTASETTCVAHATYGEPPEPARSTAT
VQECTVLTAPNCGIVNNDGAVSEGQDHGDAVHHSLDVVSQCAADTGVVDTSE
Immunization strain:
TB 40/e
DNA (codon-optimized*):
(SEQ ID NO: 85)
ATGCGGTGCTTCCGGTGGTGGCTGTACAGCGGATGGTGGTGGCTCACCTTCGGCTGC
GCCAGAACCGTGACCGTGGGCTTCGTGGCCCCTACCGTGCGGGCTCAGAGCACCGT
CGTGAGAAGCGAGCCTGCCCCCCCTAGCGAGACACGGCGGGACAACAACGACACCA
GCTACTTCAGCAGCACCAGCTTCCACAGCTCCGTGAGCCCCGCCACCTCCGTGGACC
GGCAGTTCAGACGGACCACCTACGACAGATGGGACGGCAGACGGTGGCTGCGGACC
AGATACGGCAACGCCAGCGCCTGTGTGACAGGCACCCAGTGGAGCACCAACTTTTT
CTTCAGCCAGTGCGAGCACTACCCCAGCTTCGTGAAGCTGAACGGCGTGCAGAGAT
GGACCCCCGTGCGCAGACCTATGGGCGAGGTGGCCTACTACGGCGGCTGTTGCATG
GTCGGCGGAGGGAACAGAGCCTACGTGATCCTGGTGTCCGGCTACGGCACCGCCTC
TTACGGCAATGCCCTGCGGGTGGACTTCGGCAGAGGCAACTGCACCGCCCCCAAGC
GGACCTACCCCAGACGGCTGGAACTGCACGACGGCAGAACCGACCCCAGCAGATGC
GACCCCTACCAGGTGTACTTCTACGGCCTGCAGTGCCCCGAGCAGCTGGTCATCACA
GCTCACGGCGGAGTGGGCATGAGAAGATGCCCCACCGGCAGCAGACCTACCCCCAG
CAGACCCCACAGACACGACCTGGAAAACGAGCTGCATGGCCTGTGTGTGGATCTGC
TCGTGTGCGTGCTGCTGCTGGCCCTGCTGCTGCTCGAGCTGGTGCCCATGGAAGCCG
TGAGACACCCCCTGCTGTTCTGGCGGAGAGTGGCCCTGAGCCCCAGCACCAGCAAG
GTGGACCGGGCCGTGAAGCTGTGCCTGCGGAGAATGCTGGGCCTGCCTCCTCCTCCT
TCTGTGGCCCCTCCCGGCGAGAAGAAAGAACTGCCAGCCCAGGCCGCTCTGAGCCC
TCCTCTGACCACCTGGTCCCTGCCCCCCTTCCTGAGCACCAGAATCCCCGACAGCCC
CCCTCCTCCCTATCAGCTGCGGCACGCCACAAGCCTGGTCACCGTGCCCACACTGCT
GCTGTACACCTCCAGCGACATCGGCGACACCGCCAGCGAAACCACCTGTGTGGCCC
ACGCCACCTATGGCGAGCCTCCCGAGCCTGCCAGATCCACCGCCACCGTGCAGGAA
TGCACCGTCCTGACCGCCCCTAACTGCGGCATCGTGAACAACGACGGAGCCGTGTCT
GAGGGACAGGATCACGGCGACGCTGTGCACCACAGCCTGGACGTGGTGTCCCAGTG
TGCCGCCGATACCGGCGTGGTGGATACCAGCGAG
Protein:
(SEQ ID NO: 86)
MRCFRWWLYSGWWWLTFGCARTVTVGFVAPTVRAQSTVVRSEPAPPSETRRDNNDTS
YFSSTSFHSSVSPATSVDRQFRRTTYDRWDGRRWLRTRYGNASACVTGTQWSTNFFFSQ
CEHYPSFVKLNGVQRWTPVRRPMGEVAYYGGCCMVGGGNRAYVILVSGYGTASYGN
ALRVDFGRGNCTAPKRTYPRRLELHDGRTDPSRCDPYQVYFYGLQCPEQLVITAHGGV
GMRRCPTGSRPTPSRPHRHDLENELHGLCVDLLVCVLLLALLLLELVPMEAVRHPLLFW
RRVALSPSTSKVDRAVKLCLRRMLGLPPPPSVAPPGEKKELPAQAALSPPLTTWSLPPFL
STRIPDSPPPPYQLRHATSLVTVPTLLLYTSSDIGDTASETTCVAHATYGEPPEPARSTAT
VQECTVLTAPNCGIVNNDGAVSEGQDHGDAVHHSLDVVSQCAADTGVVDTSE

Table discloses “6His” as SEQ ID NO: 6.

| | Expressed in | Antibodies in | Antibodies in |
|---|---|---|---|
| | 293T cells | immune mouse | CytoGam/ |
| | (by 6His- | sera detected | Cytotect |
| Gene | or myc-tag) | by immunoblot | (293T cells) |
| RL10 | +++ | +/− | ++ |
| RL11 | +++ | + | − |
| RL12 | − | maybe | + |
| RL13 | ++ | − | +++ |
| UL1 | +++ | ++ | − |
| UL2 | ++ | − | − |
| UL4 | +++ | − | − |
| UL5 | ++ | | − |
| UL6 | + | − | − |
| UL7 | +++ | − | + |
| UL8 | + | − | − |
| UI 9 | +++ | − | − |
| UL10 | ++ | | − |
| UL11 | ++ | | − |
| UL13 | ++ | − | − |
| UL14 | ++ | maybe | − |
| UL15A | + | may not be | − |
| UL16 | +++ | − | − |
| UL18 | +++ | − | − |
| UL20 | ++ | | − |
| UL22A | - | | − |
| UL24 | ++ | − | − |
| UL29 | +++ | | − |
| UL31 | ++ | − | − |
| UL33 | + | − | − |
| UL37 | +++ | − | − |
| UL40 | ++ | − | ++ |
| UL41A | +++ | − | − |
| UL42 | ++ | + | − |
| | Expressed in | Antibodies in | Antibodies in |
| | 293T cells | immune mouse | CytoGam/ |
| | (by 6His- | sera detected | Cytotect |
| Gene | or myc-tag, | by immunoblot | (293T cells) |
| UL50 | − | | − |
| UL78 | ++ | | − |
| UL80.5 | +++ | ++ | +++ |
| UL89 | − | − | − |
| UL105 | + | − | − |
| UL111A | +++ | − | − |
| UL116 | +++ | − | − |
| UL119 | ++ | − | +++ |
| UL120 | ++ | maybe | − |
| UL121 | ++ | − | − |
| UL122 | ++ | | +++ |
| UL124 | ++ | + | − |
| UL132 | +++ | − | +++ |
| UL133 | ++ | + | ++ |
| UL135 | ++ | − | − |
| UL136 | ++ | − | ++ |
| UL138 (Cam) | + | ++ | − |
| UL138 (Sie) | + | + | − |
| UL139 | ++ | − | +++ |
| UL140 | * | − | − |
| UL141 | + | maybe | − |
| UL142 | − | − | − |
| UL144 | + | − | − |
| UL146 | ++ | − | − |
| UL147 | ++ | | − |
| UL147A | − | − | − |
| UL148 | + | | |
| UL148A | +++ | maybe | − |
| UL148B | ++ | - | − |
| UL148C | + | − | − |
| UL148D | ++ | − | − |
| UL150 | + (MF) | | − |
| US2 | + | | − |
| US3 | − | | − |
| US6 | ++ | | − |
| US7 | ++ | + | − |
| LS8 | ++ | + | − |
| US9 | ++ | | − |
| US10 | + | − | − |
| US11 | ++ | +/− | − |
| US12 | ++ | | − |
| US13 | + | | − |
| US14 | + | − | − |
| US15 | − | − | − |
| US16 | ++ | − | − |
| US17 | ++ | − | − |
| UL47 | ++ | − | − |
| US18 | − | − | − |
| US19 | + | − | − |
| US20 | − | − | + |
| US21 | ++ | | − |
| US27 | ++ | − | ++ |
| US2S | + | | − |
| US29 | + | − | + |
| US30 | ++ | | − |
| US34 | + | − | − |
| US34A | − | − | − |

1. An immunogenic composition comprising one or more human cytomegalovirus (CMV) polypeptides selected from the group consisting of RL10, RL11, RL12, RL13, UL5, UL80.5, UL116, UL119, UL122, UL132, UL133, UL138, UL139, UL148A, and fragments thereof.

2. The immunogenic composition of claim 1, comprising human CMV polypeptide UL116 or a fragment thereof.

3. The immunogenic composition of claim 1, comprising an Fc binding protein selected from the group consisting of CMV polypeptide RL13, UL119, and a fragment thereof.

4. The immunogenic composition of claim 1, further comprising an adjuvant.

5. The immunogenic composition of claim 4, wherein the adjuvant is selected from the group comprising alum, MF59, IC31, Eisai 57, ISCOM, CpG, and pet lipid A.

6. An immunogenic complex comprising two or more human cytomegalovirus (CMV) polypeptides selected from the group consisting of RL10, RL11, RL12, RL13, UL5, UL80.5, UL116, UL119, UL122, UL132, UL133, UL138, UL139, UL148A and fragments thereof.

7. The immunogenic complex of claim 6, comprising two or more human cytomegalovirus (CMV) polypeptides selected from the group consisting of RL11, RL13 and UL119.

8. The immunogenic complex of claim 6, wherein said two CMV polypeptides are RL11 and UL119.

9. (canceled)

10. An isolated self replicating RNA comprising a sequence encoding one or more human cytomegalovirus (CMV) polypeptides selected from the group consisting of RL10, RL11, RL12, RL13, UL5, UL80.5, UL116, UL119, UL122, UL132, UL133, UL138, UL139, UL148A, and fragments thereof.

11. (canceled)

12. The isolated self replicating RNA of claim 10, comprising an alphavirus replicon.

13. An alphavirus replication particle (VRP) comprising the alphavirus replicon of claim 12.

14. An immunogenic composition comprising the self replicating RNA of claim 10.

15. An immunogenic composition comprising the VRP of claim 13.

16. The immunogenic composition of claim 14, further comprising an adjuvant.

17. A method of inducing an immune response in an individual, comprising administering to the individual an immunogenic composition of claim 1.

18-19. (canceled)

20. A method of forming a CMV protein complex, comprising delivering nucleic acids encoding two or more CMV proteins selected from the group consisting of RL10, RL11, RL12, RL13, UL5, UL80.5, UL116, UL119, UL122, UL132, UL133, UL138, UL139, and UL148A to a cell, and maintaining the cell under conditions suitable for expression of said first CMV protein and said second CMV protein, wherein a CMV protein complex is formed.

21-22. (canceled)

23. A method of inhibiting CMV entry into a cell comprising contacting the cell with the immunogenic composition of claim 1.

24. The immunogenic composition of claim 1, comprising CMV polypeptide UL80.5 or a fragment thereof.

25. The immunogenic composition of claim 1, comprising an Fc binding protein selected from the group consisting of CMV polypeptide UL119, RL11, RL12, RL13, and a fragment thereof.

26. The immunogenic composition of claim 2, further comprising a human CMV polypeptide selected from the group consisting of gB, gH, gL, gO, gM, gN, UL128, UL130, UL 131 and a fragment thereof.


SEQUENCE LISTING (88 sequences)

SEQ ID NO: 1 - DNA, 24 bp, Artificial Sequence
  Description of Artificial Sequence Synthetic oligonucleotide
ctctctacggctaacctgaatgga

SEQ ID NO: 2 - protein, 8 aa, Artificial Sequence
  Description of Artificial Sequence Synthetic peptide
DXEXNPGP

SEQ ID NO: 3 - protein, 8 aa, Foot and mouth disease virus 2A
DVESNPGP

SEQ ID NO: 4 - protein, 100 aa, Artificial Sequence
  Description of Artificial Sequence Synthetic polypeptide
KKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKKK

SEQ ID NO: 5 - DNA, 26 bp, Artificial Sequence
  Description of Artificial Sequence Synthetic oligonucleotide
gctagcggcgcgccgtcgacgccacc

SEQ ID NO: 6 - protein, 6 aa, Artificial Sequence
  Description of Artificial Sequence Synthetic 6xHis tag
HHHHHH

SEQ ID NO: 7 - DNA, 510 bp, Human cytomegalovirus
atgtatccgcgtgtaatgcacgcggtgtgctttttagcattcggcttggtaagctacgtggccttctgcgccgaaaccacggtcgccaccaactgtcttgtgaaaacagaaaatacccacctgacatgtaagtgcagtccgaataacacatctaataccggcaatggcagcaagtgccacgcggtgtgcaaatgccgggtcacagaacccattaccatgctaggcgcatactcggcctggggcgcgggctcgttcgtggccacgctgatagtcctgctggtggtcttcttcgtaatttacgcgcgcgaggaggagaaaaacaacacgggcaccgaggtagatcaatgtctggcctatcggagcctgacacgcaaaaagttggaacaacacgcggctaaaaagcagaacatctacgaacggattccataccgaccctccagacagaaagataactccccgttgatcgaaccgacgggcacagacgacgaagaggacgaggacgacgacgtc

SEQ ID NO: 8 - protein, 170 aa, Human cytomegalovirus
MYPRVMHAVCFLAFGLVSYVAFCAETTVATNCLVKTENTHLTCKCSPNNTSNTGNGSKCHAVCKCRVTEPITMLGAYSAWGAGSFVATLIVLLVVFFVIYAREEEKNNTGTEVDQCLAYRSLTRKKLEQHAAKKQNIYERIPYRPSRQKDNSPLIEPTGTDDEEDEDDDV

SEQ ID NO: 9 - DNA, 504 bp, Artificial Sequence
  Description of Artificial Sequence Synthetic polynucleotide
atgtaccccagagtgatgcacgccgtgtgctttctggccctgggcctgatcagctacgtggccgtgtgcgccgagaacaccgtgaccaccaactgcctggtcaagaccgagaatacccacctgacctgcaagtgcaaccccaacagcaccagcaccaacggcagcaagtgccacgccatgtgcaagtgcagagtgaccgagcccatcaccatgctgggcgcctattctgcctggggagccggcagctttgtggccaccctgatcgtgctgctggtcgtgttcttcgtgatctacgcccgggaggaagagaagaacaacaccggcaccgaggtggaccagtgcctggcctacagaagcctgacccggaagaagctggaacagcacgccgccaagaagcagaacatctacgagagaatcccttaccggcccagccggcagaacgacaacagccccctgatcgagcccaccggcacagacgacgaagaggacgaggacgacgacgtg

SEQ ID NO: 10 - protein, 168 aa, Artificial Sequence
  Description of Artificial Sequence Synthetic polypeptide
MYPRVMHAVCFLALGLISYVAVCAENTVTTNCLVKTENTHLTCKCNPNSTSTNGSKCHAMCKCRVTEPITMLGAYSAWGAGSFVATLIVLLVVFFVIYAREEEKNNTGTEVDQCLAYRSLTRKKLEQHAAKKQNIYERIPYRPSRQNDNSPLIEPTGTDDEEDEDDDV

SEQ ID NO: 11 - DNA, 702 bp, Human cytomegalovirus
atgcagacctacagcacccccctcacgcttgccatagtcacgtcgctgtttttgttcacaactcaaggaggttcatcgaacgccgtcgaaccaaccaaaaaacccctaaagctcgccaactaccgcgccacctgcgaggaccgtacacgtactctggttaccaggcttaacactagccatcacagcgtagtctggcaacgttatgatatctacagcagatacatgcgtcgtatgccgccactttgcatcattacagacgcctataaagaaaccacgcatcagggtggcgcaactttcacgtgcacgcgccaaaatctcacgctgtacaatcttacggttaaagatacgggagtctacctcctgcaggatcagtataccggcgatgtcgaggctttctacctcatcatccacccacgcagcttctgccgagctttggaaacgcgtcgatgcttttatccgggaccagggagagttgtggttacggattcccaagaggcagaccgagcaattatctcggatttaaaacgccagtggtccggcctctcacttcattgcgcctgggtttcgggactgatgatctttgttggcgcactggtcatctgctttctgcggtcgcaacgaatcggggaacaggacgctgaacagctgcggacggacctggatacggaacctctattgttgacggtggacggggatttggag

SEQ ID NO: 12 - protein, 234 aa, Human cytomegalovirus
MQTYSTPLTLAIVTSLFLFTTQGGSSNAVEPTKKPLKLANYRATCEDRTRTLVTRLNTSHHSVVWQRYDIYSRYMRRMPPLCIITDAYKETTHQGGATFTCTRQNLTLYNLTVKDTGVYLLQDQYTGDVEAFYLIIHPRSFCRALETRRCFYPGPGRVVVTDSQEADRAIISDLKRQWSGLSLHCAWVSGLMIFVGALVICFLRSQRIGEQDAEQLRTDLDTEPLLLTVDGDLE

SEQ ID NO: 13 - DNA, 702 bp, Artificial Sequence
  Description of Artificial Sequence Synthetic polynucleotide
atgcagacctacagcacccccctgaccctggtcatcgtgactagcctgtttctgttcacaacccagggcaacctgagcaacgccgtggagcccaccaagaagcccctgaagctggccaactaccgggccacctgcgaggacagaaccagaaccctggtcacccggctgaacaccagccaccacagcgtcgtgtggcagagatacgacatctacagccggtacatgcggagaatgccccccctgtgcatcatcaccgacgcctacaaagagacaacccaccagggcggagccaccttcacctgcacccggcagaacctgaccctgtacaacctgaccatcaaggacaccggcgtgtacctgctgcaggaccagtgtacaggcgacgtggaggccttctacctgatcatccacccccggtccttttgcagagccctggaaacccggcggtgcttttaccctggccctggcagagtggtggtcaccgacagccaggaagccgaccgggccatcatcagcgacctgaagcggcagtggagcggcctgtctctgcactgtgcctgggtgtccggcctgatgatcttcgtgggcgccctcgtgatctgcttcctgcggagccagagaatcggcgagcaggacgccgagcagctgagaaccgacctggacaccgagcctctgctgctgaccgtggacggcgacctggaa

SEQ ID NO: 14 - protein, 234 aa, Artificial Sequence
  Description of Artificial Sequence Synthetic polypeptide
MQTYSTPLTLVIVTSLFLFTTQGNLSNAVEPTKKPLKLANYRATCEDRTRTLVTRLNTSHHSVVWQRYDIYSRYMRRMPPLCIITDAYKETTHQGGATFTCTRQNLTLYNLTIKDTGVYLLQDQCTGDVEAFYLIIHPRSFCRALETRRCFYPGPGRVVVTDSQEADRAIISDLKRQWSGLSLHCAWVSGLMIFVGALVICFLRSQRIGEQDAEQLRTDLDTEPLLLTVDGDLE

SEQ ID NO: 15 - DNA, 1251 bp, Human cytomegalovirus
atgcgtacacaacatcgacggcgaaacaagtcatcgtacacgcaaataacatgcatgtttatcattttttggattctgcagaaaagcaagtgtaacaacaccactatcgctaatacttccacgtcaattacactcacaagcttgatatctactgcacaactaacatctactttacaaaccaccggaatgtctaccactacattcacatcctccgatgtcaacgccaacacatccacaggattcactgcaagctctgcaaaaagcacagacgtgatctcaactatttccaccatacccactcaaacatctacaattaacgcgactgtaatgacaacctcaccaaacggaggcatgaatttatcgacacaacatataatcagcagtaccgcgacttcgcaagcaactacatcattaccaatcaatactagtacaatggtaacaaatacaactcaaaacatcagtacaccactcccaacttgctcatcatctaatagcacattcaatgatacatcaaacaaccgtacttgtcatgaaaacagtacaatatcacaagaatctgaaacattgttgaaggcaatacaaggagacaatatcactataatacacaacctaaccaccacatcgtgctacaagacagcttggcttagacattttaatatatccacacacagaaaatacacccatcccaacataaagagtggaaaatttagtaaccattcattaaagatcctccattcgcgtgtactgtgtgagtggcagacacattacctaaaacatcactacgatttatgttttacatgcgatcagaatttatctttgtctctgtacggtcttaattttactcactctggtaaatatagctttcgatgttacaaaagtggccatccctctgaacaaaatcaaaattttaatctacaagtacatcctagaaacaacacgaacgagacacatgtgaacccctggatatgcgaagaaccaaagcacgaatgggatactttggctgctacatctgataaaccgaccagtcataaagacgatacaaccacatcatctacagatcatctataccgctataataatcattccaacacatcacacggcagacacactacgtggactttagtgttaatttgtatagcctgcattctcctatttttcgtccgacgagctctaaacaaaaaataccatccattaagggacgatatcagtgaatcagaattcatagttcgatacaatcctgagcatgaggat

SEQ ID NO: 16 - protein, 417 aa, Human cytomegalovirus
MRTQHRRRNKSSYTQITCMFIIFWILQKSKCNNTTIANTSTSITLTSLISTAQLTSTLQTTGMSTTTFTSSDVNANTSTGFTASSAKSTDVISTISTIPTQTSTINATVMTTSPNGGMNLSTQHIISSTATSQATTSLPINTSTMVTNTTQNISTPLPTCSSSNSTFNDTSNNRTCHENSTISQESETLLKAIQGDNITIIHNLTTTSCYKTAWLRHFNISTHRKYTHPNIKSGKFSNHSLKILHSRVLCEWQTHYLKHHYDLCFTCDQNLSLSLYGLNFTHSGKYSFRCYKSGHPSEQNQNFNLQVHPRNNTNETHVNPWICEEPKHEWDTLAATSDKPTSHKDDTTTSSTDHLYRYNNHSNTSHGRHTTWTLVLICIACILLFFVRRALNKKYHPLRDDISESEFIVRYNPEHED

SEQ ID NO: 17 - DNA, 1230 bp, Artificial Sequence
  Description of Artificial Sequence Synthetic polynucleotide
atgagagtgaaccggcagcggcggaacaacctgacctaccggcagaccgtgtacgtgatcctgaccttctacatcgtgcaccggggcatctgcaacagcaccgacaccaacaacagcaccagcacctccaactccaccgtgtccgacaccaatgtgtatagcacccctaacccccctagcgtgtccagcaccaccctggacaccagcaccgactcccagatcagcattgccagcaacaccatcagctccaccacaaacaccctgaccgcctacagcatcaccaccctgaatacctccacctccagcagcacactgaccgccgtgagcagcacccacacccggtccagcatcctgagcaacaacgccagctataccacctctctggacaataccaccaccgatatcaccagcagcgagagcagcatcaacgtgtccaccgtgtacaataccacctacatccccgtgaccagcctggccatcaactgcaccgccaccatcaatggcaccaacaactccagctccaagacctgtcagcaggacatcgagacaatccccgtgaagtccacccctctgaccgccgaggaaggcaccaacatcaccatccacggcaacgacacctgggactgccctgacgtggtctggtacagacactacaactggtccacccacggccaccacatctaccccaacacccactacaagaccctgatccaccggcggaagatcctgaccagccaccccatctgctacagcgacagaagcagccccaccgcctaccacgacctgtgccggtcctgcaacaagaccgagctgcggctgtacgacctgaacaccaccaactccggccggtacagcagacggtgctacaagcagtaccaccaccagggcccccacgaggacgagaacttcggcctgaccgtgaacccccggaacaacaccgacaactacaccatccccgtgtgccccagatacgtggagacacagagccaggaagatgagcaggacgacgactacaccctgagcaccaccatcaacaacaacctgatgcgcaagaccggccactacgacatcagccacggcacccacacaacctgggccctgatcctgatctgtatcgcctgcatgctgctgttcttcgtgcggagagccctgaacaagaagtaccggcccctgcgggacgatatcagcgagtccagcctggtggtgcagtatcaccccgagcacgaggac

SEQ ID NO: 18 - protein, 410 aa, Artificial Sequence
  Description of Artificial Sequence Synthetic polypeptide
MRVNRQRRNNLTYRQTVYVILTFYIVHRGICNSTDTNNSTSTSNSTVSDTNVYSTPNPPSVSSTTLDTSTDSQISIASNTISSTTNTLTAYSITTLNTSTSSSTLTAVSSTHTRSSILSNNASYTTSLDNTTTDITSSESSINVSTVYNTTYIPVTSLAINCTATINGTNNSSSKTCQQDIETIPVKSTPLTAEEGTNITIHGNDTWDCPDVVWYRHYNWSTHGHHIYPNTHYKTLIHRRKILTSHPICYSDRSSPTAYHDLCRSCNKTELRLYDLNTTNSGRYSRRCYKQYHHQGPHEDENFGLTVNPRNNTDNYTIPVCPRYVETQSQEDEQDDDYTLSTTINNNLMRKTGHYDISHGTHTTWALILICIACMLLFFVRRALNKKYRPLRDDISESSLVVQYHPEHED

SEQ ID NO: 19 - DNA, 909 bp, Human cytomegalovirus
atggactggcagtttacggttaagtggaggttactgatcatcacgttatctgaaggttgtaatgatacatgcccttgttcgtgcaactgcctcacctccaccgcctcaaccatcaaaaattcgtctgattttgtcactaacgctaccaacatttcaactactgcaaataaaaccacgcacaaaccctctaccgcctcgtcagatacatcaacaattactccaacgctgttggaatcaccgtcaagcgttacgcgaatattaacaacgttctctaccgttcatagtaccattccctggttgaataccagcaacgtaacttgcaacggtagtttgtacaccatctataaacaatctaatttaaattacgaggtaattaacgtaacagcgtatgtcggtggatacgtcactctgcaaaattgcactagaacggatacatggtatgatgtagaatggataaaatatggaactcgtacacaccaactgtgcagaattggaagttatcattcaacgtctccactaaacggcatgtgtctagactgtaacagaacctctctcaccatctacaacgtaaccgtcgaacacgctggaaaatacgttttacatcgctacattgacggtaaaaaggaaaactactatctaactgtattatggggaaccacaacatcgtctcctatacctgacaaatgcaaaacaaaagaggagtcagatcagcacaggcgcggagcgtgggacgacgtaataacaactgtaaaaaacactaacattcccctgggaattcatgctgtatgggcgggtgtagtcgtatctgtggcacttgtagccttatacatgggtagccgtcgcgcttccaggaaaccgcgttataaaaaacttcccaaatatgatccagatgagttttggactaaaacc

SEQ ID NO: 20 - protein, 303 aa, Human cytomegalovirus
MDWQFTVKWRLLIITLSEGCNDTCPCSCNCLTSTASTIKNSSDFVTNATNISTTANKTTHKPSTASSDTSTITPTLLESPSSVTRILTTFSTVHSTIPWLNTSNVTCNGSLYTIYKQSNLNYEVINVTAYVGGYVTLQNCTRTDTWYDVEWIKYGTRTHQLCRIGSYHSTSPLNGMCLDCNRTSLTIYNVTVEHAGKYVLHRYIDGKKENYYLTVLWGTTTSSPIPDKCKTKEESDQHRRGAWDDVITTVKNTNIPLGIHAVWAGVVVSVALVALYMGSRRASRKPRYKKLPKYDPDEFWTKT

SEQ ID NO: 21 - DNA, 882 bp, Artificial Sequence
  Description of Artificial Sequence Synthetic polynucleotide
atgcactggcacctggccatcacctggacagtgatcatcagcaccttcagcgagtgctgcaaccagacctgtccctgcagctgcgtgtgcgtgaacagcaccaccgtgaacatctccaccaacgagacaaccagcaaggccatcacccccaccgccaccaccaataccgccaagaccacctccagcctggtgattacaacacccagcagcgtgacaatcagcaaggccgtgagcacagccgccagcagcaccatcctgagccagaccaaccggtcccacaccagcaacgtgatcacaacccctaagacccgcttcgagtacaacatcaccggctacgtgggccaggaagtgaccttcaacttcagcggcagcttctggtcctacatcgagtggttccggtacagcagccccggctggctgtatagcagcgaacccatctgcaccgtgaccaacagctaccaccacaccttccccagaggcaccctgtgcttcgactgcaacatgaccaagttcgtgatctacgacctgaccctgaacgacagcggcaaatacgtggtgaagcggacccggcacgacaaccagtacgaggaagcctgctacaatctgacagtgatctacgccaacaccaccgccatcgtgaccaaccggacctgtgaccggcggcagaccaagaacaccgataccaccaaccacggcatcggcaagcacatcatcgagacaatcaagaaggccaacatccccctgggcattcatgccgtgtgggccggcattgtggtgtctgtggccctgatcgccctgtacatgggcaaccggcggaggcccagaaagccccggtacacccggctgcccaagtacgaccccgacgagttctggaccaagacc

SEQ ID NO: 22 - protein, 294 aa, Artificial Sequence
  Description of Artificial Sequence Synthetic polypeptide
MHWHLAITWTVIISTFSECCNQTCPCSCVCVNSTTVNISTNETTSKAITPTATTNTAKTTSSLVITTPSSVTISKAVSTAASSTILSQTNRSHTSNVITTPKTRFEYNITGYVGQEVTFNFSGSFWSYIEWFRYSSPGWLYSSEPICTVTNSYHHTFPRGTLCFDCNMTKFVIYDLTLNDSGKYVVKRTRHDNQYEEACYNLTVIYANTTAIVTNRTCDRRQTKNTDTTNHGIGKHIIETIKKANIPLGIHAVWAGIVVSVALIALYMGNRRRPRKPRYTRLPKYDPDEFWTKT

SEQ ID NO: 23 - DNA, 498 bp, Human cytomegalovirus
atgtttctaggctactctgactgtgtagatcccggctttgctgtatatcgtgtatctagatcacgcttgaagctcgtgttgtcttttgtgtggttggtcggtttgcgtctccatgattgtgccacgttcgaatcctgctgttacgacatcaccgaggcggagagtaacaaggctatatcaagggacgaagcagcattcacctccagcgtgagcacccgcacaccgtccctggtgatcgcgccgcctcctgaccgatcgatgctgttatcacgggaggaagaactcgttccgtggagtcgtctcatcatcactaagcagttctacggaggcctgattttccacaccacctgggttaccggcttcgttttgctaggactcttgacgcttttcgccagcctgtttcgcgtgccgcaatccatctgtcgtttctgcatagaccgtctccgggacatcgcccgtcctttgaaataccgctatcaacgtctcgtcgccaccgtg

SEQ ID NO: 24 - protein, 166 aa, Human cytomegalovirus
MFLGYSDCVDPGFAVYRVSRSRLKLVLSFVWLVGLRLHDCATFESCCYDITEAESNKAISRDEAAFTSSVSTRTPSLVIAPPPDRSMLLSREEELVPWSRLIITKQFYGGLIFHTTWVTGFVLLGLLTLFASLFRVPQSICRFCIDRLRDIARPLKYRYQRLVATV

SEQ ID NO: 25 - DNA, 498 bp, Artificial Sequence
  Description of Artificial Sequence Synthetic polynucleotide
atgtttctgggctacagcgactgcgtggaccccggcttcgccgtgtaccgggtgtccagatcccggctgaagctggtgctgtccttcgtgtggctcgtgggcctgagactgcacgactgcgccaccttcgagagctgctgctacgacatcaccgaggccgagagcaacaaggccatcagccgggacgaggccgtgttcaccagcagcgtgtccaccagaacccccagcctggccattgccccccctcccgatagaagtatgctgctgtcccgggaagaggaactggtgccctggtctagactgatcatcaccaagcagttctacggcggcctgatcttccacaccacctgggtgaccggctttgtgctgctgggcctgctgaccctgttcgccagcctgttccgggtgccccagagcatctgccggttctgcatcgaccggctgcgggatatcgccagacccctgaagtacagataccagagactggtcgccaccgtg

SEQ ID NO: 26 - protein, 166 aa, Artificial Sequence
  Description of Artificial Sequence Synthetic polypeptide
MFLGYSDCVDPGFAVYRVSRSRLKLVLSFVWLVGLRLHDCATFESCCYDITEAESNKAISRDEAVFTSSVSTRTPSLAIAPPPDRSMLLSREEELVPWSRLIITKQFYGGLIFHTTWVTGFVLLGLLTLFASLFRVPQSICRFCIDRLRDIARPLKYRYQRLVATV

SEQ ID NO: 27 - DNA, 1119 bp, Human cytomegalovirus
atgtcgcaccctctgagtgctgcggttcccgccgctacggctcctccaggtgctaccgtggcaggtgcgtcgccggctgtgccgtctctagcgtggcctcacgacggagtttatttacccaaagacgcttttttctcgctacttggggccagtcgctcggcagcgcccgtcatgtatcccggtgccgtagcggctcctccttctgcttcgccagcaccgttgcctttgccgtcttatcccgcgccctacggcgcccccgtcgtgggttacgaccagttggcgacacgtcactttgcggaatacgtggatccccattatcccgggtggggtcggcgttacgagcccgcgccgcctttgcattcggcttgtcccgtgccgccgccaccatcaccagcctattaccgtcggcgcgattctccgggcggtatggatgaaccaccgtccggatgggagcgttacgacggtggtcaccgtggtcagtcgcagaagcagcaccgtcacgggggcagcggtggacacaacaaacgccgtaaggaagctgcggcggcgtcgtcgtcgtcctcggacgaagacttgagtttccccggcgaggccgagcacggccgggcgcgaaagcgtctaaaaagtcacgtcaatagcgacggtggaagtggcgggcacgcgggttccaatcagcagcagcaacaacgttacgatgaactgcgggatgccattcacgagctgaaacgcgatctgtttgccgcgcggcagagttctacgttactttcggcggctctccccgctgcggcctcttcctccccaactactactaccgtgtgtactcccaccggcgagctgacgagtggcggaggagaaacacccacggcacttctatccggaggtgccaaggtagctgagcgcgctcaggccggcgtggtgaacgccagttgccgcctcgctaccgcgtcgggttctgaggcggcaacggccgggccctcgacggcaggttcttcttcctgcccggctagtgtcgtgttagccgccgctgctgcccaagccgccgcagcttcccagagcccgcccaaagacatggtagatctgaatcggcggatttttgtggctgcgctcaataagctcgag

SEQ ID NO: 28 - protein, 373 aa, Human cytomegalovirus
MSHPLSAAVPAATAPPGATVAGASPAVPSLAWPHDGVYLPKDAFFSLLGASRSAAPVMYPGAVAAPPSASPAPLPLPSYPAPYGAPVVGYDQLATRHFAEYVDPHYPGWGRRYEPAPPLHSACPVPPPPSPAYYRRRDSPGGMDEPPSGWERYDGGHRGQSQKQHRHGGSGGHNKRRKEAAAASSSSSDEDLSFPGEAEHGRARKRLKSHVNSDGGSGGHAGSNQQQQQRYDELRDAIHELKRDLFAARQSSTLLSAALPAAASSSPTTTTVCTPTGELTSGGGETPTALLSGGAKVAERAQAGVVNASCRLATASGSEAATAGPSTAGSSSCPASVVLAAAAAQAAAASQSPPKDMVDLNRRIFVAALNKLE

SEQ ID NO: 29 - DNA, 1119 bp, Artificial Sequence
  Description of Artificial Sequence Synthetic polynucleotide
atgagccatcctctgtctgccgctgtgcctgctgctacagcccctcctggcgctacagtggctggcgcctctcctgctgtgccttctctggcctggcctcacgatggcgtgtacctgcccaaggacgccttctttagcctgctgggcgcctctagatctgccgcccctgtgatgtatcctggcgccgtggccgctcctccttctgcctctcccgccccactgcctctgcctagctaccctgccccttacggcgctcccgtcgtgggatacgaccagctggccaccagacacttcgccgagtacgtggaccctcactaccctggctggggcagaagatatgagcctgccccccctctgcatagcgcctgccccgtgcctcctcctcctagccccgcctactacagaagaagagacagccctggcgggatggatgagcctccttccggctgggagagatacgatggcggccaccggggacagagccagaagcagcacagacacggcgggtccgggggacacaacaagcggcggaaagaggccgcagccgcttccagctccagctccgacgaggacctgagctttcctggcgaggccgagcacggcagagcccggaagagactgaagtcccacgtgaacagcgatggcggatctggcggccatgccggctctaatcagcagcagcagcagagatacgacgagctgcgggacgccatccacgagctgaagcgggacctgttcgccgccagacagtccagcaccctgctgtctgcagctctcccagccgctgccagcagctctcctaccaccaccaccgtgtgcacccctaccggcgagctgacaagcggagggggcgagacacctaccgctctgctgtccggcggagccaaagtggccgaaagggcccaggctggcgtggtcaatgcttcctgtagactggccacagccagcggctctgaagccgccacagccggccctagcacagccggcagcagctcttgtcctgcctctgtggtgctggcagctgctgcagctcaggctgctgccgcctcccagagcccccccaaggacatggtggacctgaaccggcggatcttcgtggccgccctgaacaagctggaa

SEQ ID NO: 30 - protein, 373 aa, Artificial Sequence; identical to SEQ ID NO: 28
  Description of Artificial Sequence Synthetic polypeptide

SEQ ID NO: 31 - DNA, 939 bp, Human cytomegalovirus
atgaagcggcggcggcgatggcggggctggttgcttttcctggccctgtgcttttgcttactgtgtgaagcggtggaaaccaacgcgaccaccgttaccagtaccaccgctgccgccgccacgacaaacactaccgtcgccaccaccggtaccactactacctcccctaacgtcacttcaaccacgagtaacaccgtcatcactcccaccacggtttcctcggtcagcaatctgacatccagcgccacgtcgattcccatctcaacgtcaacggtttctggaacaagaaacacaaggaataataataccacaaccatcggtacgaacgttacttccccctccccttctgtatccatacttaccaccgtgacaccggccgcgacttctaccacctccaacaacggggatgtaacatccgactacactccaacttttgacctggaaaacattaccaccacccgcgctcccacgcgtcctcccgcccaggacctttgtagccataacctgtcaatcatcctgtacgaagaggaatctcagagcagcgtagacattgcggtggatgaagaagagccagaactggaggacgacgacgagtacgacgaactgtggttccccctctacttcgaggctgagtgcaacctaaattacacgctacaatacgtcaatcacagttgtgattacagcgtgcgccagtcgtctgtctcattccccccgtggcgcgacatcgactcagttaccttcgtacccaggaacctctccaactgtagcgcccacggtctggccgtcatcgtcgcgggtaaccaaacctggtacgtgaatccgtttagcctggctcacctgctggatgcaatatataacgttttagggatcgaagacctgagcgccaactttcggcgccaactggctccttaccgtcacactctcatcgtgccgcagact

SEQ ID NO: 32 - protein, 313 aa, Human cytomegalovirus
MKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQT

SEQ ID NO: 33 - DNA, 939 bp, Artificial Sequence
  Description of Artificial Sequence Synthetic polynucleotide
atgaagcggcggagaagatggcggggctggctgctgttcctggccctgtgcttctgtctgctgtgcgaggccgtggagacaaacgccaccaccgtgaccggaacaacagccgccgctgccaccaccaataccactgtcgccaccaccggcaccaccaccacctcccccaacgtgaccagcaccacaagcaacaccgtgaccacccctaccaccgtgtccagcgtgtccaacctgacctccagcacaacctccatccccatcagcaccagcaccgtgtccggcacccggaacaccggcaacaacaataccaccaccatcgggactaacgctacctctcccagcccttccgtgagcatcctgaccacagccaccccagccgctacctccacaaccagcaacaacggcgacgtgacctccgactacacccccaccttcgacctggaaaacatcaccaccacaagagcccctaccagaccccctgcccaggatctgtgcagccacaacctgagcatcatcctgtacgaggaagagtcccagagcagcgtggatatcgccgtggacgaggaagaacccgagctggaagatgacgacgagtacgacgagctgtggttccccctgtacttcgaggccgagtgcaacctgaactacaccctgcagtacgtgaaccacagctgcgactacagcgtgcggcagtcctccgtgagcttccccccctggcgggacatcgacagcgtgaccttcgtgccccggaacctgagcaattgcagcgcccacggcctggctgtgatcgtggccggcaaccagacttggtacgtgaatcccttcagcctggcccacctgctggacgccatctacaacgtgctgggcatcgaggacctgagcgccaacttcagacggcagctggccccctacagacacaccctgatcgtgccccagacc

SEQ ID NO: 34 - protein, 313 aa, Artificial Sequence
  Description of Artificial Sequence Synthetic polypeptide
MKRRRRWRGWLLFLALCFCLLCEAVETNATTVTGTTAAAATTNTTVATTGTTTTSPNVTSTTSNTVTTPTTVSSVSNLTSSTTSIPISTSTVSGTRNTGNNNTTTIGTNATSPSPSVSILTTATPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQT

SEQ ID NO: 35 - DNA, 1035 bp, Human cytomegalovirus
atgtgttccgtgctggcgatcgcgctcgtagttgcgctcttgggcgacatgcacccgggagtgaaaagtagcaccacaagcgccgtcacttcccctagtaataccaccgtcacgtctactacgtcaataagtacctctaacaacgtcagttctgctgtcaccaccacggtacaaacctctacctcgtccgcctccacctccgtgatagccacgacgcagaaagaggggcacctgtatactgtgaattgcgaagccagctacagctacgaccaagtgtctctaaacgccacctgcaaagttatcctgttgaataataccaaaaatccagacattttatcagttacttgttatgcacggacagactgcaagggtcccttcactcaggtgggatatcttagcgcttttccctccaacgataaaggaaaactacatctctcctacaacgctactgctcaagagctgcttatctcgggactcaggccgcaggagaccactgagtacacgtgctctttcttcagttggggccgccatcacaacgccacttgggaccttttcacctatcccatttacgccgtgtacgggactcgcttgaacgctaccacgatgcgggtccgcgtgctgcttcaggaacacgaacactgcttgctcaacggtagcagcctctatcaccccaacagcaccgtgcatctgcatcagggcgaccagctcattccgccgtggaatattagtaacgtgacgtataacggacaacggttacgcgagtttgtcttctacctcaacggcacgtatactgtcgtgcgtctccacgtccagatcgcgggccgaagttttaccaccacctacgtgtttatcaagagcgacccgctgttcgaggaccggctgctggcctacggcgtgctggctttcctggtgttcatggtaattattcttttgtacgtgacctacatgctggcgcgccggcgggactggtcctataagagactggaggagcccgttgaagaaaagaaacacccggtgccctacttcaagcagtgg

SEQ ID NO: 36 - protein, 345 aa, Human cytomegalovirus
MCSVLAIALVVALLGDMHPGVKSSTTSAVTSPSNTTVTSTTSISTSNNVSSAVTTTVQTSTSSASTSVIATTQKEGHLYTVNCEASYSYDQVSLNATCKVILLNNTKNPDILSVTCYARTDCKGPFTQVGYLSAFPSNDKGKLHLSYNATAQELLISGLRPQETTEYTCSFFSWGRHHNATWDLFTYPIYAVYGTRLNATTMRVRVLLQEHEHCLLNGSSLYHPNSTVHLHQGDQLIPPWNISNVTYNGQRLREFVFYLNGTYTVVRLHVQIAGRSFTTTYVFIKSDPLFEDRLLAYGVLAFLVFMVIILLYVTYMLARRRDWSYKRLEEPVEEKKHPVPYFKQW

SEQ ID NO: 37 - DNA, 1032 bp, Artificial Sequence
  Description of Artificial Sequence Synthetic polynucleotide
atgtgcagcgtgctggccattgccctggtggtggctctcctgggcgacatgcaccccagagtgaagtccagcaccacctccgccgtgaccagccccagcaacaccaccgtgacctccaccacctccatcagcaccagcaacaacgtcactagcgctgtcacaaccaccgtgcagaccagcacaagcagcgccagcaccagcgtgatcgccaccacccagaaagagggccacctgtacaccgtgaactgcgaggccagctacagctacgaccaggtgtccctgaacgccacctgcaaagtgatcctgctgaacaacaccaagaaccccgacatcctgagcgtgacctgctacgccagaaccgactgcaagggccccttcacccaggtcggctacctgagcgccttccccagcaacgacaagggcaagctgcacctgagctacaacgccaccgcccaggaactgctgatcagcggcctgaggccccaggaaaccaccgagtacacctgcagctttttcagctggggcagacaccacaatgccacctgggacctgttcacctaccccatctacgccgtgtacggcaccagactgaatgccaccaccatgagagtgcgggtgctgctgcaggaacacgagcactgcctgctgaacggcagcagcctgtaccaccccaacagcacagtgcacctgcatcagggaaaccagctgattccaccctggaacatcagcaacgtgacctacaacggccagcggctgcgggagttcgtgttctacctgaacggcacctacaccgtcgtgcggctgcatgtgcagatcgccggcagatccttcaccaccacctatgtgttcatcaagagcgaccccctgttcgaggacagactgctggcctacggggtgctggccttcctggtgttcatggtcatcatcctgctgtacgtgacatacatgctggccagacggcgggactggtcctacaagcggctggaagaacccgtggaggaaaagaagcaccccgtcccttacttcaagcag

SEQ ID NO: 38 - protein, 344 aa, Artificial Sequence
  Description of Artificial Sequence Synthetic polypeptide
MCSVLAIALVVALLGDMHPRVKSSTTSAVTSPSNTTVTSTTSISTSNNVTSAVTTTVQTSTSSASTSVIATTQKEGHLYTVNCEASYSYDQVSLNATCKVILLNNTKNPDILSVTCYARTDCKGPFTQVGYLSAFPSNDKGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFEDRLLAYGVLAFLVFMVIILLYVTYMLARRRDWSYKRLEEPVEEKKHPVPYFKQ

SEQ ID NO: 39 - DNA, 1740 bp, Human cytomegalovirus
atggagtcctctgccaagagaaagatggaccctgacaaccctgacgagggcccttcctccaaggtgccacggcccgagacacccgtgaccaaggccacgacgttcctgcagactatgttaaggaaggaggttaacagtcagctgagcctgggagacccgctgttcccagaattggccgaagaatctctcaaaacctttgaacaagtgaccgaggattgcaacgagaaccccgaaaaagatgtcctggcagaactcggtgacatcctcgcccaggctgtcaatcatgccggtatcgattccagtagcaccggccccacgctgacaacccactcttgcagcgttagcagcgcccctcttaacaagccgacccccaccagcgtcgcggttactaacactcctctccccggggcatccgctactcccgagctcagcccgcgtaagaaaccgcgcaaaaccacgcgtcctttcaaggtgattattaaaccgcccgtgcctcccgcgcctatcatgctgcccctcatcaaacaggaagacatcaagcccgagcccgactttaccatccagtaccgcaacaagattatcgataccgccggctgtatcgtgatctctgatagcgaggaagaacagggtgaagaagtcgaaacccgcggtgctaccgcgtcttccccttccaccggcagcggcacgccgcgagtgacctctcccacgcacccgctctcccagatgaaccaccctcctcttcccgatcccttgggccggcccgatgaagatagttcctcttcgtcttcctcctcctgcagttcggcttcggactcggagagtgagtccgaggagatgaaatgcagcagtggcggaggagcatccgtgacctcgagccaccatgggcgcggcggttttggtggcgcggcctcctcctctctgctgagctgcggccatcagagcagcggcggggcgagcaccggaccccgcaagaagaagagcaaacgcatctccgagttggacaacgagaaggtacgcaatatcatgaaagataagaacacccccttctgcacacccaacgtgcagactcggcggggtcgcgtcaagattgacgaggtgagccgcatgttccgcaacaccaatcgctctcttgagtacaagaacctgcccttcacgattcccagtatggaccaggtgttagatgaggccatcaaagcttgcaaaaccatgcaggtgaacaacaagggcatccagatcatctacacccgcaatcatgaggtgaagagtgaggtggatgcggtgcggtgtcgcctgggcaccatgtgcaacctggccctctccactcccttcctcatggagcacaccatgcctgtgacacacccacccgaagtggcgcagcgcacggccgatgcttgtaacgaaggcgtcaaagccgcgtggagcctcaaagaattgcacacccaccaattatgcccccgttcttccgattaccgcaacatgatcatccacgctgccacccccgtggacctgttgggcgctctcaacctgtgcctacccctgatgcaaaagtttcccaaacaggtcatggtgcgcatcttctccaccaaccagggtgggttcatgctgcctatctacgagacggccgcgaaggcctacgccgtggggcagtttgagcagcccaccgagacccctcccgaagacctggacaccctgagcctggccatcgaggcagccatccaggacctgaggaacaagtctcag

SEQ ID NO: 40 - protein, 580 aa, Human cytomegalovirus
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGPTLTTHSCSVSSAPLNKPTPTSVAVTNTPLPGASATPELSPRKKPRKTTRPFKVIIKPPVPPAPIMLPLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFTIPSMDQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYAVGQFEQPTETPPEDLDTLSLAIEAAIQDLRNKSQ

SEQ ID NO: 41 - DNA, 1740 bp, Artificial Sequence
  Description of Artificial Sequence Synthetic polynucleotide
atggaaagcagcgccaagcggaagatggaccccgacaaccccgatgagggccccagcagcaaggtgcccagacccgagacacctgtgaccaaggccaccacctttctgcagaccatgctgcggaaagaagtgaacagccagctgtccctgggcgaccctctgtttcccgagctggccgaggaaagcctgaaaaccttcgagcaggtcaccgaggactgcaacgagaaccccgagaaggacgtgctggctgaactgggcgatattctggcccaggccgtgaaccacgccggcatcgatagcagcagcaccggccacaccctgaccacccacagctgcagcgtgtccagcgcccctctgaacaagcccacccccacaagcgtggccgtgaccaacacacctctgcctggcgcctctgccacacccgagctgtccccccggaagaagcccagaaagaccacccggcccttcaaagtgatcatcaagccccccgtgccccctgctcctatcatgctgcccctgctgattaagcaggaagatatcaagcccgagcccgacttcaccatccagtaccggaacaagatcatcgacaccgccggctgcatcgtgatcagcgacagcgaggaagaacagggcgaggaagtggagacaagaggcgccaccgccagcagccctagcacaggcagcggcacccctagagtgaccagccccacccaccccctgagccagatgaaccacccccccctgcctgatcctctgggcagacccgacgaggatagcagctccagctcctctagctcttgcagcagcgccagtgatagcgaatcagagtccgaagagatgaagtgcagctctggcggcggagccagcgtgacaagcagccaccacggcagaggcggatttggcggagccgcctcttctagcctgctgtcctgtggccaccagtcctccggcggagcctctaccggccccagaaagaagaagtccaagcggatcagcgagctggacaacgagaaagtgcggaacatcatgaaggacaagaacacccccttttgcacccccaacgtgcagaccagacggggcagagtgaagatcgacgaggtgtcccggatgttcagaaacaccaaccggtccctggaatacaagaacctgcccttcatgatccccagcatgcaccaggtgctggacgaggccatcaaggcctgcaagaccatgcaggtcaacaacaagggcatccagatcatctacacccggaaccacgaagtgaagtccgaggtggacgccgtgagatgcagactgggcaccatgtgcaacctggccctgagcaccccctttctgatggaacacaccatgcccgtgacccaccctccagaggtggcccagagaaccgccgatgcctgcaacgaaggcgtgaaggccgcctggtccctgaaagagctgcacacacaccagctgtgccccagaagcagcgactaccgcaacatgatcattcacgccgccacccctgtggatctgctgggcgccctgaacctgtgcctgcccctgatgcagaaattccccaagcaggtcatggtccggatcttcagcaccaaccagggcggcttcatgctgcctatctacgagacagccgccaaggcctacgacgtgggccagttcgagcagcctaccgagacaccccccgaggacctggataccctgagcctggccatcgaggctgctatccaggacctgcggaacaagagc

SEQ ID NO: 42 - protein, 580 aa, Artificial Sequence
  Description of Artificial Sequence Synthetic polypeptide
MESSAKRKMDPDNPDEGPSSKVPRPETPVTKATTFLQTMLRKEVNSQLSLGDPLFPELAEESLKTFEQVTEDCNENPEKDVLAELGDILAQAVNHAGIDSSSTGHTLTTHSCSVSSAPLNKPTPTSVAVTNTPLPGASATPELSPRKKPRKTTRPFKVIIKPPVPPAPIMLPLLIKQEDIKPEPDFTIQYRNKIIDTAGCIVISDSEEEQGEEVETRGATASSPSTGSGTPRVTSPTHPLSQMNHPPLPDPLGRPDEDSSSSSSSSCSSASDSESESEEMKCSSGGGASVTSSHHGRGGFGGAASSSLLSCGHQSSGGASTGPRKKKSKRISELDNEKVRNIMKDKNTPFCTPNVQTRRGRVKIDEVSRMFRNTNRSLEYKNLPFMIPSMHQVLDEAIKACKTMQVNNKGIQIIYTRNHEVKSEVDAVRCRLGTMCNLALSTPFLMEHTMPVTHPPEVAQRTADACNEGVKAAWSLKELHTHQLCPRSSDYRNMIIHAATPVDLLGALNLCLPLMQKFPKQVMVRIFSTNQGGFMLPIYETAAKAYDVGQFEQPTETPPEDLDTLSLAIEAAIQDLRNKS

SEQ ID NO: 43 - DNA, 810 bp, Human cytomegalovirus
atgccggccccgcggggtccccttcgcgcaacattcctggccctggtcgcgttcgggttgctgcttcagatagacctcagcgacgctacgaatgtgaccagcagcacaaaagtccctactagcaccagcagcagaaatagcgtcgacaatgccacgagtagcggacccacgaccgggatcaacatgaccaccacccacgagtcttccgttcacagcgtgcgcaatgacgaaatcatgaaagtgctggctatcctcttctacatcgtgacaggcacctccattttcagcttcatagcggtactgatcgcggtagtttactcctcgtgttgcaagcacccgggccgctttcgtttcgccgacgaagaagccgtcaacctgttggacgacacggacgacagtggcggtggcagcccgtttggcagcggttcccgacgaggttctcagatccccgccggattttgttcctcgagcccttatcagcggttggaaactcgggactgggacgaggaggaggaggcgtccgcggcccgcgagcgcatgaaacatgatcctgagaacgtcatctatttcagaaaggatggcaacttggacacgtcgttcgtgaatcccaattatgggagaggctcgcctttgaccatcgaatctcacctctcggacaatgaggaagaccccatcaggtactacgtctcggtgtacgatgaactgaccgcctcggaaatggaagaaccttcgaacagcaccagctggcagattcccaaactaatgaaagttgccatgcaacccgtctcgctcagagatcccgagtacgac

SEQ ID NO: 44 - protein, 269 aa, Human cytomegalovirus
MPAPRGPLRATFLALVAFGLLLQIDLSDATNVTSSTKVPTSTSSRNSVDNATSSGPTTGINMTTTHESSVHSVRNDEIMKVLAILFYIVTGTSIFSFIAVLIAVVYSSCCKHPGRFRFADEEAVNLLDDTDDSGGGSPFGSGSRRGSQIPAGFCSSSPYQRLETRDWDEEEEASAARERMKHDPENVIYFRKDGNLDTSFVNPNYGRGSPLTIESHLDNEEDPIRYYVSVYDELTASEMEEPSNSTSWQIPKLMKVAMQPVSLRDPEYD

SEQ ID NO: 45 - DNA, 810 bp, Artificial Sequence
  Description of Artificial Sequence Synthetic polynucleotide
atgcctgcccctagaggcctgctgagagccaccttcctggtgctcgtggcctttggcctgctgctgcacatggacttcagcgacgccacaaacatgaccagcagcaccaacgtgcccacctccacctccagccggaacaccgtggagagcaccacaagcagcgagcccaccaccgaaaccaacatgaccaccgccagagaaagcagcgtgcacgacgcccggaacgacgagatcatgaaggtgctggccatcctgttctacatcgtgaccggcaccagcatcttcagctttatcgccgtgctgatcgccgtggtgtactctagttgctgcaagcaccccggcagattcagattcgccgacgaggaagccgtgaatctgctggacgacaccgacgatagcggcggcagcagcccttttggcagcggcagcagaagaggctctcagatccctgccggcttctgttctagcagcccctaccagcggctggaaacccgggactgggacgaggaagaggaagccagcgccgccagggaaagaatgaagcatgaccctgagaatgtgatctacttccggaaggacggcaacctggacaccagcttcgtgaaccccaactacggcagaggcagccccctgaccatcgagtcccacctgagcgacaacgaagaggaccccatccggtactacgtgtccgtgtacgacgagctgaccgccagcgagatggaagaacccagcaacagcaccagctggcagatccccaagctgatgaaggtcgccacccagagcgtgtccctgagggaccccgagtacgac

SEQ ID NO: 46 - protein, 270 aa, Artificial Sequence
  Description of Artificial Sequence Synthetic polypeptide
MPAPRGLLRATFLVLVAFGLLLHMDFSDATNMTSSTNVPTSTSSRNTVESTTSSEPTTETNMTTARESSVHDARNDEIMKVLAILFYIVTGTSIFSFIAVLIAVVYSSCCKHPGRFRFADEEAVNLLDDTDDSGGSSPFGSGSRRGSQIPAGFCSSSPYQRLETRDWDEEEEASAARERMKHDPENVIYFRKDGNLDTSFVNPNYGRGSPLTIESHLSDNEEDPIRYYVSVYDELTASEMEEPSNSTSWQIPKLMKVATQSVSLRDPEYD

SEQ ID NO: 47 - DNA, 768 bp, Human cytomegalovirus
atgggttgcgacgtgcacgatccttcgtggcaatgccaatggggcgttcccacgattattgtggcctggataacatgcgcggccctgggaatttggtgtttggtaggatcaccgaatacgttttcgggacccggcatcgcagccgtagtcggctgttctgttttcatgattttcctctgcgcgtatctcatccgttaccgggaattcttcaaggactccgtaatcgacgtcttcacctgccgatgggtgcgctactgcagctgcagctgtaagtgcagctgcaaatgcatttcgggtccttgtagccgctgctgttcagcgtgttacaaggagacgatgatttacgacatggttcaatatggtcatcgacggcgtcccggacacggcgacgatcccgacagggtgatctgcgagatagtcgagagtcccccggtttcggcgccgacagtattcgtccccccgccgtcggaggagtcccaccagcccgtcatcccaccgcagccgccaacaccgacatcggaacccaaaccgaagaaaggtagggcgaaagataaaccgaagagcaaaccgaaggacaaacctccgtgcgagccgacggtgagttcacaaccaccgtcgcagccgacggcgatgcccggcggtccgcccgacgcgtctccccccgccatgccgcagatgccacccggcgtggccgaggcggtacaagctgccgtgcaggcggccatggccgcggctctacaacaacagcagcagcatcagaccggaacg

SEQ ID NO: 48 - protein, 256 aa, Human cytomegalovirus
MGCDVHDPSWQCQWGVPTIIVAWITCAALGIWCLVGSPNTFSGPGIAAVVGCSVFMIFLCAYLIRYREFFKDSVIDVFTCRWVRYCSCSCKCSCKCISGPCSRCCSACYKETMIYDMVQYGHRRRPGHGDDPDRVICEIVESPPVSAPTVFVPPPSEESHQPVIPPQPPTPTSEPKPKKGRAKDKPKSKPKDKPPCEPTVSSQPPSQPTAMPGGPPDASPPAMPQMPPGVAEAVQAAVQAAMAAALQQQQQHQTGT

SEQ ID NO: 49 - DNA, 771 bp, Artificial Sequence
  Description of Artificial Sequence Synthetic polynucleotide
atgggctgtgacgtgcaggaccccagctgtcagtgtcagtggggcgtgcctgccatcatcgtgatctggatgatctgtgccgccctgggcatttggtgtctggccggcagcagcgccaatatcttcagcggccctggcattgctgccgtggtcgtgtgcagcgtgttcatgatctttctgtgcgcctacctgatccggtacagagagttcttcaaggacagcatcatcgacatcctgacctgtagatgggtgcgctactgctcctgctcctgcaagtgcagctgtaagtgtatcagcggaccctgctccagatgctgtagcgcctgctacaaagaaaccatgatctacgacatggtgcagtacggccacagaagaaggcctggccacggcgacgaccccgacagagtgatctgcgagatcgtggagagccctcccgtgtccgcccctaccgtgttcgtgcctcctccctccgaggaatctcaccagcccgtgatcccccctcagcctcctacccctaccagcgagcccaagcccaagaagggcagagccaaggacaagcccagaggcagacctaagaacaagcccccctgcgagcctacagtgtccagccagccccctagccagccaacagccatgcctggcggccctccagatgcccctcctcccgccatgcctcagatgcctccaggcgtggccgaagctgtgcaggccgccgtgcagacagctgtggccgctgctctgcagcagcaacagcagcaccagaccggcacc

SEQ ID NO: 50 - protein, 257 aa, Artificial Sequence
  Description of Artificial Sequence Synthetic polypeptide
MGCDVQDPSCQCQWGVPAIIVIWMICAALGIWCLAGSSANIFSGPGIAAVVVCSVFMIFLCAYLIRYREFFKDSIIDILTCRWVRYCSCSCKCSCKCISGPCSRCCSACYKETMIYDMVQYGHRRRPGHGDDPDRVICEIVESPPVSAPTVFVPPPSEESHQPVIPPQPPTPTSEPKPKKGRAKDKPRGRPKNKPPCEPTVSSQPPSQPTAMPGGPPDAPPPAMPQMPPGVAEAVQAAVQTAVAAALQQQQQHQTGT

SEQ ID NO: 51 - DNA, 510 bp, Human cytomegalovirus
atggacgatctgccgctgaacgtcgggttacccatcatcggcgtgatgctcgtgctgatcgtggccattctctgctatctagcttaccattggcacgacaccttcaaactggtgcgcatgtttttgagctaccgctggctgatccgctgttgcgagctgtacggggaatacgagcgccggttcgcggacctgtcgtcgctgggcctcggcgccgtacggcgggagtcggacagacgataccgtttctccgaacggcccgatgagatcttggtccgttgggaggaagtgtcttcccagtgcagctacgcgtcgtcgcggataacagaccgccgcgcgggttcatcgtcttcgtcgtcggtccacgtcgctaaccagagaaacagcgtgcctccgccggacatggcggtgacggcgccgctgaccgacgtcgatctgttgaaacccgtgacgggatccgcgacgcagttcaccaccgtagccatggtacattatcatcaagaatacacgtga

SEQ ID NO: 52 - protein, 169 aa, Human cytomegalovirus
MDDLPLNVGLPIIGVMLVLIVAILCYLAYHWHDTFKLVRMFLSYRWLIRCCELYGEYERRFADLSSLGLGAVRRESDRRYRFSERPDEILVRWEEVSSQCSYASSRITDRRAGSSSSSSVHVANQRNSVPPPDMAVTAPLTDVDLLKPVTGSATQFTTVAMVHYHQEYT

SEQ ID NO: 53 - DNA, 507 bp, Artificial Sequence
  Description of Artificial Sequence Synthetic polynucleotide
atggacgacctgcccctgaacgtgggcctgcccatcatcggcgtgatgctggtgctgatcgtggccatcctgtgctacctggcctaccactggcacgacaccttcaagctcgtgcggatgttcctgagctaccggtggctgatccggtgttgcgagctgtacggcgagtacgagcggagattcgccgatctgagcagcctgggcctgggcgccgtgagaagagagagcgaccggcggtacagattcagcgagcggcccgacgaaatcctcgtgcgctgggaagaggtgtccagccagtgcagctacgccagcagccggatcacagacagaagggccggcagcagcagctctagcagcgtgcacgtggccaaccagagaaacagcgtgccccctcccgatatggccgtgaccgcccctctgaccgacgtggacctgctgaagcctgtgaccggcagcgccacccagtttaccaccgtggccatggtgcactaccaccaggaatacacc

SEQ ID NO: 54 - protein, 169 aa, Artificial Sequence; identical to SEQ ID NO: 52
  Description of Artificial Sequence Synthetic polypeptide

SEQ ID NO: 55 - DNA, 240 bp, Human cytomegalovirus
atgagttccagcgacaatctcgatccttggattcccgtgtgcgtcgtggtggtcatgacctccgtagtcctgttcgcaggtctgcacgtgtacttgtggtacgttcggcggcagctggtggcgttctgcctggagaaggtgtgcgttcgctgctgcggaaaagatgagacgacgccgctagtggaggatgccgaaccgccggcggagctggagatggtggaagtgtcggacgagtgttac

SEQ ID NO: 56 - protein, 80 aa, Human cytomegalovirus
MSSSDNLDPWIPVCVVVVMTSVVLFAGLHVYLWYVRRQLVAFCLEKVCVRCCGKDETTPLVEDAEPPAELEMVEVSDECY

SEQ ID NO: 57 - DNA, 240 bp, Artificial Sequence
  Description of Artificial Sequence Synthetic polynucleotide
atgagcagcagcgacaacctggacccctggattcccgtgtgcgtggtggtggtcatgactagcgtggtgctgtttgccggcctgcatgtgtacctctggtacgtgcggagacagctggtcgccttctgcctggaaaaagtgtgcgtgcggtgctgcggcaaggacgagacaacccccctggtggaggatgccgagcctcccgccgagctggaaatggtggaggtgtccgacgagtgctac

SEQ ID NO: 58 - protein, 80 aa, Artificial Sequence; identical to SEQ ID NO: 56
  Description of Artificial Sequence Synthetic polypeptide

SEQ ID NO: 59 - DNA, 666 bp, Human cytomegalovirus
atggcttccgacgtgggttctcatcctctgacagttacacgattccgctgcaaagtgcatcatgtgtacaataaactgttgattttagctttgtttgcccccgtgattctggaatccgttatctacgtgtccgggccacagggagggaacgttaccctgatatccaacttcacttcaaacatcagcgtacggtggtttcgctgggacggcaacgatagccatctcatttgcttttacaaacgtggagaaggtctttctacgccctatgtgggtttaagcttaagttgtgcggctaaccagatcaccatcttcaacctcacgttaaacgactccggtcgttacggagcagaaggttttacgagaagcggcgaaaatgaaacgtttctgtggtataatttgaccgtgaaacccaaacctttggaaactactccagctagtaacgtaacaaccatcgtcacgacgacatcgacggtgaccggcgcgaaaagtaacgttacggggaacgccggtttagcaccacaactacgtgtcgtcgctggattctccaatcagacgcctttggaaaacaacacgcacatggccttggtaggtgttgtcgtgtttctagccctaatagttgtttgtattatggggtggtggaagttgttgtgtagtaaaccaaagtta

SEQ ID NO: 60 - protein, 222 aa, Human cytomegalovirus
MASDVGSHPLTVTRFRCKVHHVYNKLLILALFAPVILESVIYVSGPQGGNVTLISNFTSNISVRWFRWDGNDSHLICFYKRGEGLSTPYVGLSLSCAANQITIFNLTLNDSGRYGAEGFTRSGENETFLWYNLTVKPKPLETTPASNVTTIVTTTSTVTGAKSNVTGNAGLAPQLRVVAGFSNQTPLENNTHMALVGVVVFLALIVVCIMGWWKLLCSKPKL

SEQ ID NO: 61 - DNA, 666 bp, Artificial Sequence
  Description of Artificial Sequence Synthetic polynucleotide
atggcctctgatgtgggcagccaccccctgaccgtgacccggttccggtgcagagtgcaccacgtgtacaacaagctgctgatcctggccctgttcgcccccgtgatcctggaaagcgtgatctacgtgtccggccctcagggcggcaatgtgaccctgatcagcaacttcaccagcaacatcagcgtgcggtggttcagatgggacggcaacgacagccacctgatctgcttctacaagcggggcgagggcctgagcacaccttacgtgggcctgagcctgagctgcgccgccaaccagatcaccatcttcaacctgaccctgaacgacagcggcagatacggcgccgagggcttcaccagaagcggcgagaacgagacattcctgtggtacaatctgaccgtgaagcccaagcccctggaaaccacccctgccagcaacgtgaccaccatcgtgaccacaaccagcaccgtgaccggcgccaagtccaacgtgaccggcaatgcctctctggccccccagctgagagctgtggccggctttagcaaccagacccccctggaaaacaacacccacatggccctggtcggcgtggtggtgtttctggccctgatcgtggtctgcatcatggggtggtggaagctgctgtgcagcaagcccgaactg

SEQ ID NO: 62 - protein, 222 aa, Artificial Sequence
  Description of Artificial Sequence Synthetic polypeptide
MASDVGSHPLTVTRFRCRVHHVYNKLLILALFAPVILESVIYVSGPQGGNVTLISNFTSNISVRWFRWDGNDSHLICFYKRGEGLSTPYVGLSLSCAANQITIFNLTLNDSGRYGAEGFTRSGENETFLWYNLTVKPKPLETTPASNVTTIVTTTSTVTGAKSNVTGNASLAPQLRAVAGFSNQTPLENNTHMALVGVVVFLALIVVCIMGWWKLLCSKPEL

SEQ ID NO: 63 - DNA, 663 bp, Human cytomegalovirus
atgaacaaattcagcaacactcgtatcggcttcacttgcgcggttgtggctccgcggactttaattctgacgcttggactcctgtgtatgaggatcaggagtttattatcttctcctgccgagacgacggtaacaaccgccggcgtgacgtccgctcacggtccgttatgtccgctcgtgttccagggttgggcgtacgccgtgtaccaccaaggcgacatggccctcatgacactcgacgtgtactgctgccgccagacctccaacaacaccgccgtcgcgttctcgcgtcatcttgccgttaacacgctgttgatcgaagtgggtaacaacactcgccgccgtgcagacggagtctcctgcctggaccattttcgcgcgcaacaccaggattgcccggcccagacggtgcacgtgcgcggcgtaaacgaaagcgcttttggactcacccatctgcagtcctgttgcctgaacgagcattcacaactctcggagcgggtggcctaccatctgaagctgcgacccgccacgttcggtctggagacctgggccatgtacactgtgggcattctggccctggggtcgttctcctccttctattcccagatcgctaggagcctgggggttctgcccaacgatcatcactacgccttgaaaaaggct

SEQ ID NO: 64 - protein, 221 aa, Human cytomegalovirus
MNKFSNTRIGFTCAVVAPRTLILTLGLLCMRIRSLLSSPAETTVTTAGVTSAHGPLCPLVFQGWAYAVYHQGDMALMTLDVYCCRQTSNNTAVAFSRHLAVNTLLIEVGNNTRRRADGVSCLDHFRAQHQDCPAQTVHVRGVNESAFGLTHLQSCCLNEHSQLSERVAYHLKLRPATFGLETWAMYTVGILALGSFSSFYSQIARSLGVLPNDHHYALKKA

SEQ ID NO: 65 - DNA, 663 bp, Artificial Sequence
  Description of Artificial Sequence Synthetic polynucleotide
atgaacaagttcagcaacacccggatcggcttcacctgtgccgtgatggcccccagaaccctgatcctgaccctgggcctgctgtgcatgcggatcagatccctgctgtgctcccctgccgagacaaccgtgaccaccgctggcgccatgtctgcccacggccccagatgccctctggtgttccagggctgggcctacgccgtgtaccatcagggcgacatggctctgatgaccctggatgtgtactgctgtcggcagaccagcagcaacaccgtggtggccttcagccaccaccccgccgacaacaccctgctgatcgaagtgggcaacaacaccagacggcacgtggacggcatcagctgccaggaccacttcagagcccagcaccaggattgccctgcccagacagtgcacgtgcggggcgtgaatgagagcgccttcggcctgacccacctgcagagctgctgcctgaacgagcacagccagctgtccgagagagtggcctaccacctgaagctgaggcccgccacctttggcctggaaacctgggccatgtacaccgtgggcatcctggctctgggcagcttcagcagcttctacagccagatcgccagatctctcggcgtgctgcccaacgatcaccactacgccctgaagaaggcc

SEQ ID NO: 66 - protein, 221 aa, Artificial Sequence
  Description of Artificial Sequence Synthetic polypeptide
MNKFSNTRIGFTCAVMAPRTLILTLGLLCMRIRSLLCSPAETTVTTAGAMSAHGPRCPLVFQGWAYAVYHQGDMALMTLDVYCCRQTSSNTVVAFSHHPADNTLLIEVGNNTRRHVDGISCQDHFRAQHQDCPAQTVHVRGVNESAFGLTHLQSCCLNEHSQLSERVAYHLKLRPATFGLETWAMYTVGILALGSFSSFYSQIARSLGVLPNDHHYALKKA

SEQ ID NO: 67 - DNA, 720 bp, Human cytomegalovirus
atgtcagtcaagggcgtggagatgccagaaatgacgtgggacttggacgttggaaataaatggcggcgtcgaaaggccctgagtcgcattcaccggttctgggaatgtcgactacgggtgtggtggctgagtgacgccggcgtaagagaaaccgacccaccgcgtccccgacgccgcccgacttggatgaccgcggtgtttcacgttatctgtgccgttttgcttacgcttatgattatggccatcggcgcgctcatcgcgtacttaagatattaccaccaggacagttggcgagacatgctccacgatctattttgcggctgtcattatcctgagaagtgccgtcggcaccacgagcggcagagaagcagacggcgagccatggatgtgcccgacccggaactcggcgacccggcccgccggccgttgaacggggccatgtactacggcagcggctgtcgcttcgacacggtggaaatggtggacgagacgagacccgcgccgccggcgctgtcatcgcccgaaaccggcgacgatagcaacgacgacgcggttgccggcggaggtgctggcggggtaacatcatccgcgactcgtacgacgtcgtcgaacgcgctgctgccagaatggatggatgcggtacatgtggcggtccaagccgccgttcaagcgaccgtgcaagtaagtggcccgcgggagaacgccgtatctcccgctacg

SEQ ID NO: 68 - protein, 240 aa, Human cytomegalovirus
MSVKGVEMPEMTWDLDVGNKWRRRKALSRIHRFWECRLRVWWLSDAGVRETDPPRPRRRPTWMTAVFHVICAVLLTLMIMAIGALIAYLRYYHQDSWRDMLHDLFCGCHYPEKCRRHHERQRSRRRAMDVPDPELGDPARRPLNGAMYYGSGCRFDTVEMVDETRPAPPALSSPETGDDSNDDAVAGGGAGGVTSSATRTTSSNALLPEWMDAVHVAVQAAVQATVQVSGPRENAVSPAT

SEQ ID NO: 69 - DNA, 720 bp, Artificial Sequence
  Description of Artificial Sequence Synthetic polynucleotide
atgagcgtgaagggcgtggagatgcccgagatgacctgggacctggacgtgggcaacaagtggcggcggagaaaggccctgagcagaatccaccggttctgggagtgccggctgagagtgtggtggctctccgatgccggcgtgagagagacagacccccccagacccagacgcagacccacctggatgaccgccgtgttccacgtgatctgcgccgtgctgctgaccctgatgatcatggccatcggcgccctgatcgcctacctgcggtactaccaccaggacagctggcgggacatgctgcacgacctgttctgcggctgccactaccccgagaagtgcagacggcaccacgagcggcagcggagaaggcggagagccatggacgtgcccgaccctgaactgggcgaccctgccagacgacccctgaacggcgccatgtactacggcagcggctgcagattcgacaccgtggagatggtggacgagacaagacctgccccccctgccctgtctagccccgagacaggcgacgacagcaacgatgatgccgtggcaggaggcggagctggcggagtcaccagcagcgccaccagaaccacctccagcaacgccctgctgcccaagtggatggatgccgtgcatgtggccgtgcaggccgctgtgcaggctacagtgcaggtgtccggccctagagaaaacgccgtgagccctgccacc

SEQ ID NO: 70 - protein, 240 aa, Artificial Sequence
  Description of Artificial Sequence Synthetic polypeptide
MSVKGVEMPEMTWDLDVGNKWRRRKALSRIHRFWECRLRVWWLSDAGVRETDPPRPRRRPTWMTAVFHVICAVLLTLMIMAIGALIAYLRYYHQDSWRDMLHDLFCGCHYPEKCRRHHERQRRRRRAMDVPDPELGDPARRPLNGAMYYGSGCRFDTVEMVDETRPAPPALSSPETGDDSNDDAVAGGGAGGVTSSATRTTSSNALLPKWMDAVHVAVQAAVQATVQVSGPRENAVSPAT

SEQ ID NO: 71 - DNA, 405 bp, Human cytomegalovirus
atgctgtggatattaattttatttgcactcgccgcatcggcgagtgaaaccactacaggtaccagctctaattccagtcaatctactagtgctaccgccaacacgaccgtatcgacatgtattaatgcctctaacggcagtagctggacagtaccacagctcgcgctgcttgccgctagcggctggacattatctggactccttctcttatttacctgctgcttttgctgcttttggttagtacgtaaaatctgcagctgctgcggcaattcctccgagtcagagagcaaaacaacccacgcgtacaccaatgccgcattcacttcttccgacgcgacgttacccatgggcactacagggtcgtacactcccccacaggacggctcatttccacctccgcctcgg

SEQ ID NO: 72 - protein, 135 aa, Human cytomegalovirus
MLWILILFALAASASETTTGTSSNSSQSTSATANTTVSTCINASNGSSWTVPQLALLAASGWTLSGLLLLFTCCFCCFWLVRKICSCCGNSSESESKTTHAYTNAAFTSSDATLPMGTTGSYTPPQDGSFPPPPR

SEQ ID NO: 73 - DNA, 432 bp, Artificial Sequence
  Description of Artificial Sequence Synthetic polynucleotide
atgctgtggattctggtgctgttcgccctggccgccagcgccagcgagacaaccaccggcaccagcagcaacagcagccagagcaccagctccagcagcacctccagcaatagcaccgccacccccacaagcgccagcatccagtgcgtggagagcttcggcggcagcaattggacagtggcccagctggccctgtttgctgccagcggctggacactgagcggcctgctgctgctgttcacctgttgcttttgctgcttctggctggtccggaagatctgcagctgctgcggcaacagctccgagagcgagagcaagaccacccacgcctacaccaacgccgccttcaccagctccgatgccaccctgcctatgggcaccaccggcagctacacccctccccaggacggcagcttccccccacctcctaga

SEQ ID NO: 74 - protein, 144 aa, Artificial Sequence
  Description of Artificial Sequence Synthetic polypeptide
MLWILVLFALAASASETTTGTSSNSSQSTSSSSTSSNSTATPTSASIQCVESFGGSNWTVAQLALFAASGWTLSGLLLLFTCCFCCFWLVRKICSCCGNSSESESKTTHAYTNAAFTSSDATLPMGTTGSYTPPQDGSFPPPPR

SEQ ID NO: 75 - DNA, 762 bp, Human cytomegalovirus
atgcaggcgcaggaggctaacgcgctgctgctctcccgcatggaggctctcgagtggttcaaaaagttcaccgtatggctgcgcgtgtacgccatcttcatctttcagctggctttcagcttcggcttgggaagcgttttttggttggggttcccacaaaaccgcaacttttgcgtcgagaactacagcttctttctcaccgtgctcgtgcccatcgtctgcatgttcatcacgtacacgttgggcaacgaacaccctagtaacgccacggtgcttttcatctatctgttggccaacagcctgacggcggccatcttccaaatgtgctctgaaagccgcgtactagtaggttcctacgtgatgaccctggcgttgtttatctcctttacggggctggcgtttctaggtggccgtgaccgacgtcgctggaaatgcatcagctgcgtctacgtggtgatgctgctttcgttcctcacgctcgctctgctaagcgacgccgattggctgcagaagatagtggtgacgttgtgcgccttctctatcagcttctttttgggtattctggcctacgacagtctcatggtcatctttttctgcccacctaaccaatgcatccgtcacgccgtctgtctctacctggacagcatggccatctttctcacgttgttgctcatgctctcgggtccccgttggattagtctttcggacggcgcgcctttggacaacgggactttgacagccgccagtacgacggggaagtcc

SEQ ID NO: 76 - protein, 254 aa, Human cytomegalovirus
MQAQEANALLLSRMEALEWFKKFTVWLRVYAIFIFQLAFSFGLGSVFWLGFPQNRNFCVENYSFFLTVLVPIVCMFITYTLGNEHPSNATVLFIYLLANSLTAAIFQMCSESRVLVGSYVMTLALFISFTGLAFLGGRDRRRWKCISCVYVVMLLSFLTLALLSDADWLQKIVVTLCAFSISFFLGILAYDSLMVIFFCPPNQCIRHAVCLYLDSMAIFLTLLLMLSGPRWISLSDGAPLDNGTLTAASTTGKS

SEQ ID NO: 77 - DNA, 762 bp, Artificial Sequence
  Description of Artificial Sequence Synthetic polynucleotide
atgcaggcccaggaagccaacgccctgctgctgtcccggatggaagccctggaatggttcaagaagttcaccgtctggctgcgggtgtacgccatcttcatcttccagctggccttcagctttggcctgggcagcgtgttctggctgggcttccctcagaaccggaacttctgcgtggagaactacagcttcttcctgaccgtgctggtgcccatcgtgtgcatgttcatcacctacaccctgggcaacgagcaccccagcaacgccaccgtgctgttcatctacctgctggccaacagcctgaccgccgccatcttccagatgtgcagcgagagcagagtgctcgtgggcagctacgtgatgaccctggcactgttcatcagcttcaccggcctggcctttctgggcggcagagacagacggcggtggaagtgcatcagctgcgtgtacgtggtcatgctgctgtcttttctgacactggccctgctgtccgacgccgactggctgcagaaaatcgtggtcaccctgtgcgccttcagcatcagcttttttctgggcatcctggcctacgacagcctgatggtcatcttcttttgcccccccaaccagtgcatcagacacgccgtgtgcctgtacctggacagcatggccatctttctgactctgctgctgatgctgtccggccccagatggatcagcctgagcgacggcgctcccctggataatggcaccctgacagccgccagcaccacaggcaagagc

SEQ ID NO: 78 - protein, 254 aa, Artificial Sequence; identical to SEQ ID NO: 76
  Description of Artificial Sequence Synthetic polypeptide

SEQ ID NO: 79 - DNA, 1083 bp, Human cytomegalovirus
atgaccacctctacaaaccaaaccttaacacaggtgagcaacatgacaaatcacaccttgaacaacaccgaaatctatcagctgttcgagtacactcggttgggggtatggttgatgtgcatcgtgggcacgtttctgaacgtgctggtgatcaccaccatcatgtactaccgtcgtaagaagaaatctccgagcgatacttacatctgcaacctggctatagccgatctgctgattgtcgtcggcctgccgttttttctagaatatgccaagcatcaccctaaactcagccgagaggtggtttgttcgggactcaacgcttgtttctacatctgtctttttgccggcgtttgttttctcatcaacctgtcgatggatcgctactgcgtcattgtttggggtgtagaattgaaccgcgtgcgaaataacaagcgggccacctgttgggtggtgattttttggatactagccgtgcttatggggatgccacattacctgatgtacagccataccaacaacgagtgtgttggtgaattcgctaacgagacttcgggttggttccccgtgtttttgaacaccaaagttaacatttgcggctacctggcgcccattgcgctgatggcgtacacgtacaaccgtatggtgcggtttatcattaactacgttggtaaatggcacatgcagacgctccacgttcttttggttgtggttgtgtcttttgccagcttttggtttcctttcaacctggcgctatttttagaatccatccgtcttctggcgggagtgtacaatgacacacttcaaaacgttattatcttctgtctatacgtcggtcagtttttggcctacgttcgcgcttgtctgaatcctgggatctacatcctagtaggcactcaaatgaggaaggacatgtggacaaccctaagggtattcgcctgttgctgcgtgaagcaggagataccttaccaggacattgatattgagctacaaaaggacatacaaagaagggccaaacacaccaaacgtacccattatgacagaaaaaatgcacctatggagtccggggaggaggaatttctattg

SEQ ID NO: 80 - protein, 361 aa, Human cytomegalovirus
MTTSTNQTLTQVSNMTNHTLNNTEIYQLFEYTRLGVWLMCIVGTFLNVLVITTIMYYRRKKKSPSDTYICNLAIADLLIVVGLPFFLEYAKHHPKLSREVVCSGLNACFYICLFAGVCFLINLSMDRYCVIVWGVELNRVRNNKRATCWVVIFWILAVLMGMPHYLMYSHTNNECVGEFANETSGWFPVFLNTKVNICGYLAPIALMAYTYNRMVRFIINYVGKWHMQTLHVLLVVVVSFASFWFPFNLALFLESIRLLAGVYNDTLQNVIIFCLYVGQFLAYVRACLNPGIYILVGTQMRKDMWTTLRVFACCCVKQEIPYQDIDIELQKDIQRRAKHTKRTHYDRKNAPMESGEEEFLL

SEQ ID NO: 81 - DNA, 1086 bp, Artificial Sequence
  Description of Artificial Sequence Synthetic polynucleotide
atgaccacctccaccaacaaccagaccctgacccaggtgtccaacatgaccaaccacaccctgaacagcaccgagatctaccagctgttcgagtacacccggctgggcgtgtggctgatgtgcatcgtgggcacctttctgaacgtgctggtcatcaccaccatcctgtactaccggcggaagaagaagtcccccagcgacacctacatctgcaacctggccgtggccgacctgctgatcgtcgtgggcctgcccttcttcctggaatacgccaagcaccaccccaagctgtcccgggaggtcgtgtgtagcggcctgaacgcctgcttctacatctgcctgttcgccggcgtgtgcttcctgatcaacctgagcatggaccggtactgcgtgatcgtgtggggcgtggagctgaacagagtgcggaacaacaagcgggccacctgctgggtggtcatcttctggattctggccgtgctgatgggcatgcctcactacctgatgtacagccacaccaacaacgagtgcgtgggcgagttcgccaacgagacaagcggctggttccccgtgttcctgaacaccaaagtgaacatctgcggctacctggcccctatcgccctgatggcctacacctacaaccggatggtccggttcatcatcaactacgtgggcaagtggcacatgcagaccctgcacgtgctgctggtcgtggtggtgtccttcgccagcttctggttccccttcaacctggccctgttcctggaaagcatccggctgctggctggcgtgtacaacgacaccctgcagaacgtgatcatcttctgcctgtacgtgggccagttcctggcctatgtgcgggcctgcctgaacccaggcatctacatcctcgtgggcacacagatgcggaaggatatgtggaccaccctgcgggtgttcgcctgctgctgcgtgaagcaggaaatcccctaccaggacatcgacatcgagctgcagaaggacatccagcggagagccaagaacaccaagcggacccactacgacagaaagcacgcccccatggaaagcggcgaggaagagttcctgctg

SEQ ID NO: 82 - protein, 362 aa, Artificial Sequence
  Description of Artificial Sequence Synthetic polypeptide
MTTSTNNQTLTQVSNMTNHTLNSTEIYQLFEYTRLGVWLMCIVGTFLNVLVITTILYYRRKKKSPSDTYICNLAVADLLIVVGLPFFLEYAKHHPKLSREVVCSGLNACFYICLFAGVCFLINLSMDRYCVIVWGVELNRVRNNKRATCWVVIFWILAVLMGMPHYLMYSHTNNECVGEFANETSGWFPVFLNTKVNICGYLAPIALMAYTYNRMVRFIINYVGKWHMQTLHVLLVVVVSFASFWFPFNLALFLESIRLLAGVYNDTLQNVIIFCLYVGQFLAYVRACLNPGIYILVGTQMRKDMWTTLRVFACCCVKQEIPYQDIDIELQKDIQRRAKNTKRTHYDRKHAPMESGEEEFLL

SEQ ID NO: 83 - DNA, 1386 bp, Human cytomegalovirus
atgcggtgtttccgatggtggctctacagtgggtggtggtggctcacgtttggatgtgctcggaccgtgacggtgggtttcgtcgcgcccacggtccgggcacaatcaaccgtggtccgctctgagccggctccgccgtcggaaacccgacgagacaacaatgacacgtcttacttcagcagcacctctttccattcttccgtgtcccctgccacctcagtggaccgtcaatttcgacggaccacgtacgaccgttgggacggtcgacgttggctgcgcacccgctacgggaacgccagcgcctgcgtgacgggcacccaatggagcaccaacttttttttctctcagtgtgagcactaccctagtttcgtgaaactcaacggggtgcagcgctggacacctgttcggagacctatgggcgaggttgcctactacgggggttgttgtatggtgggcgggggtaatcgtgcgtacgtgatactcgtgagcggttacgggaccgccagctacggcaacgctttacgcgtggattttgggcgcggcaactgcacggcgccgaaacgcacctaccctcggcgcttggaactgcacgatggccgcacagaccctagccgttgcgatccctaccaagtatatttctacggtctgcagtgtcctgagcaactggttatcaccgcccacggcggcgtgggtatgcgccgctgtcctaccggctctcgtcccaccccgtcccggccccaccggcatgacttggagaacgagctacatggtctgtgtgtggatcttctggtgtgcgtccttttattagctctgctgctgttggagctcgttcccatggaagccgtgcgtcacccgctgcttttctggcgacgcgtggcgttatcgccgtccacttccaaggtggatcgcgccgtcaagctgtgtcttcggcgcatgctgggtctgccgccgccaccgtcagtcgcaccacctggggaaaagaaggagctaccggctcaggcggccttgtcgccgccactgaccacctggtcactaccgccgtttctgtccacgcggatacctgacagtccgccgccaccgtaccagcttcgtcacgccacgtcactagtgacggtacccacgctgctgttatatacgtcatccgacatcggtgacacagcttcagaaacaacgtgtgtggcgcacgctacttatggggaacccccggagcccgctcgatcgacggctacggttcaggaatgtacggttcttaccgccccgaattgcggcatcgtcaacaacgacggcgcggtctctgaaggccaagaccatggagatgcggttcaccatagcctggatgtggtttcccagtgtgctgctgatactggggttgttgacacctccgag

SEQ ID NO: 84 - protein, 462 aa, Human cytomegalovirus
MRCFRWWLYSGWWWLTFGCARTVTVGFVAPTVRAQSTVVRSEPAPPSETRRDNNDTSYFSSTSFHSSVSPATSVDRQFRRTTYDRWDGRRWLRTRYGNASACVTGTQWSTNFFFSQCEHYPSFVKLNGVQRWTPVRRPMGEVAYYGGCCMVGGGNRAYVILVSGYGTASYGNALRVDFGRGNCTAPKRTYPRRLELHDGRTDPSRCDPYQVYFYGLQCPEQLVITAHGGVGMRRCPTGSRPTPSRPHRHDLENELHGLCVDLLVCVLLLALLLLELVPMEAVRHPLLFWRRVALSPSTSKVDRAVKLCLRRMLGLPPPPSVAPPGEKKELPAQAALSPPLTTWSLPPFLSTRIPDSPPPPYQLRHATSLVTVPTLLLYTSSDIGDTASETTCVAHATYGEPPEPARSTATVQECTVLTAPNCGIVNNDGAVSEGQDHGDAVHHSLDVVSQCAADTGVVDTSE

SEQ ID NO: 85 - DNA, 1386 bp, Artificial Sequence
  Description of Artificial Sequence Synthetic polynucleotide
atgcggtgcttccggtggtggctgtacagcggatggtggtggctcaccttcggctgcgccagaaccgtgaccgtgggcttcgtggcccctaccgtgcgggctcagagcaccgtcgtgagaagcgagcctgccccccctagcgagacacggcgggacaacaacgacaccagctacttcagcagcaccagcttccacagctccgtgagccccgccacctccgtggaccggcagttcagacggaccacctacgacagatgggacggcagacggtggctgcggaccagatacggcaacgccagcgcctgtgtgacaggcacccagtggagcaccaactttttcttcagccagtgcgagcactaccccagcttcgtgaagctgaacggcgtgcagagatggacccccgtgcgcagacctatgggcgaggtggcctactacggcggctgttgcatggtcggcggagggaacagagcctacgtgatcctggtgtccggctacggcaccgcctcttacggcaatgccctgcgggtggacttcggcagaggcaactgcaccgcccccaagcggacctaccccagacggctggaactgcacgacggcagaaccgaccccagcagatgcgacccctaccaggtgtacttctacggcctgcagtgccccgagcagctggtcatcacagctcacggcggagtgggcatgagaagatgccccaccggcagcagacctacccccagcagaccccacagacacgacctggaaaacgagctgcatggcctgtgtgtggatctgctcgtgtgcgtgctgctgctggccctgctgctgctcgagctggtgcccatggaagccgtgagacaccccctgctgttctggcggagagtggccctgagccccagcaccagcaaggtggaccgggccgtgaagctgtgcctgcggagaatgctgggcctgcctcctcctccttctgtggcccctcccggcgagaagaaagaactgccagcccaggccgctctgagccctcctctgaccacctggtccctgccccccttcctgagcaccagaatccccgacagcccccctcctccctatcagctgcggcacgccacaagcctggtcaccgtgcccacactgctgctgtacacctccagcgacatcggcgacaccgccagcgaaaccacctgtgtggcccacgccacctatggcgagcctcccgagcctgccagatccaccgccaccgtgcaggaatgcaccgtcctgaccgcccctaactgcggcatcgtgaacaacgacggagccgtgtctgagggacaggatcacggcgacgctgtgcaccacagcctggacgtggtgtcccagtgtgccgccgataccggcgtggtggataccagcgag

SEQ ID NO: 86 - protein, 462 aa, Artificial Sequence; identical to SEQ ID NO: 84
  Description of Artificial Sequence Synthetic polypeptide

SEQ ID NO: 87 - protein, 293 aa, Human cytomegalovirus
MHWHLAITWTVIILTFSECYNQTCPCPCICVNSTTVSISTSETTSKNITPTTTTNSKKTTSSIATTTPSLVTTGKVVSTAASSTIISQTNRSHTSNAITTPKTRFEYNITGYVGQEVTLNFTGSWNYIQWFRYGSPGWLYSSEPICTVTSNYHHTFPRGALCFDCDMTKLLIYDLTLNDSGKYVVKRTRHDNQYEEACYSLTVIFANTTSIVTNRTCDRKRTENTDTTNHEIGKHIIETIKKANIPLGIHAVWAGIVVSVALIALYMGNRRRPRKPRYTRLPKYDPDESWTKT

SEQ ID NO: 88 - protein, 303 aa, Human cytomegalovirus; identical to SEQ ID NO: 20